SEQUENCE LISTING (228 sequences)

SEQ ID NO: 1 - DNA, 403 bp, Cannabis sativa
  source 1..403: mol_type = other DNA
  misc_difference 202: note = g or a
cggctacgctttcgccggggatagctctcttcccgcgccggtcatattttccggcgttcctccggcgacaaccgctactgccaccgcttggtcaccttccttatcgtctgctctctacaaagtcgatgggtggggcgcaccttacttcgccgtcaactcttccggcaacgtcgccgttcgccctcacggcgctggaactttngcgcaccaggagattgatttgctgaaaattgtaaagaaggtttcggatccgaaatctaagggcgggttaggtttgccccttccgctcgttattcggcttcctgatgtgcttaagaaccgccttgagtctctccaggcggcgttcgatttcgcaatccagtcgcaggactatgaaaaccattaccagggtgtttaccctgtg

SEQ ID NO: 2 - DNA, 403 bp, Cannabis sativa
  source 1..403: mol_type = other DNA
  misc_difference 202: note = g or a
tcaagaaataagaattaactaaatattgccacttaacctagtaaaattaagagcagtttcacgtgttaaaattaaataaaataatgaactcaacagaaacttaaaccacaatacctccaacttgttggggggtattggttttacattctctactttcaatgtacaacaaccaacagtatctgcttcatcatcatcctgcaangcatgagatcataatgtcagtgatgccttcagttaacagctaggggaacttaaagatgatgtagagaaacatgttgagcaacttaaatgatgcaatgtagcagattcgagcaacaaatataaatcaaacacctttcatatttctttttccttaacaagggcacaaataaacaatgacaaaaatgttaaactgaagagctgt

SEQ ID NO: 3 - DNA, 403 bp, Cannabis sativa
  source 1..403: mol_type = other DNA
  misc_difference 202: note = a or c
catgcaaatacatacatatacatatgtgatttttttttatgtatactatatatataggtcatggggtgatgtgaatgagtgcttgggaagcaagagatcaagtatcataaataatggaggggagttaggccatgaatatcgtgatcatcatcatcatcagcatcggcatattggggtttcaacgatgaagatgaagaagatnaaagggagaaggaaagtgagagaaccaaggttttgctttaagaccatgagcgaggtcgatgtgcttgatgatggttacaagtggagaaagtacggacagaaagtggtcaagaacacacagcatcccaggtacctaattaatatccatttattcattaatttataatattaacaactctaacaatgccattaatattaaagc

SEQ ID NO: 4 - DNA, 403 bp, Cannabis sativa
  source 1..403: mol_type = other DNA
  misc_difference 202: note = a or g
gattgcactgtgaaagggattcgaagtaacggccggatcctcatgacggtcttgatcaatatgagcaattaatgttctagggtttaggcctcttgattgatggtggtgatgggcagccgccattgaaacggaattactagggttttctaattccctggtcgtagtagtgtttgatgtagtggttgccatgagaaaatgggangtaggctttcgttcttggattatttgggtggtgctagtagtagtactactactcattataagctctggcttattattcatgtcacaaaatgagtagtgtggttttgatgagaagaagaagctgggtgatgttgggttaatgagttcccagttccaaggagcaggtaataaacctagaaagtcgtcctcggactttgaccca

SEQ ID NO: 5 - DNA, 403 bp, Cannabis sativa
  source 1..403: mol_type = other DNA
  misc_difference 202: note = a or g
tccaatctttgggagcaacactgtaagatgtaacagtgacaccttcactattagtgacttcaaaggataggggttgattttttaaatcagcatttatgtgccagttttggccccagttccttcccattgaaagccaaccagttcttgaacccttaattttcacagcagatacgtctccagcaccggcaacattgctgattancaccgaaatgaagatacttgaaccgtcaattgtgaatcgtattcctccttcctttctgcactttattctgttacagaagtcaaattaactagttgtcaacatgccacattttttcctaatagatttaatcacaccgaacatagttttcgtcatcaatatgccacatttacaacaacattgtcatctcatctttttcactat

SEQ ID NO: 6 - DNA, 403 bp, Cannabis sativa
  source 1..403: mol_type = other DNA
  misc_difference 202: note = g or a
cgaaaagcctatctgaaatcttagttctacagataggacactctgaacaagctaatgaacaagatttacatactgcaaaacaaaatgataaatatgattagttttaatttaatgttaaaagtgaaatcaagcatgtaggattcataggaatgatgatgactgcttacaacaaaaatggcgacacggcaaaagaattgcagcngtcggggattcaaaacatactttacacatgtgagaattggcatctccattccccatttgtttcagctccttttccttcatctcttgcattcgagcctgaaaacaacatacatcttgagtttgagataccttaacactttttcgagaactttttagtttcttgatcaacaaatgcattttccttgtggatttaaaattatgc

SEQ ID NO: 7 - DNA, 403 bp, Cannabis sativa
  source 1..403: mol_type = other DNA
  misc_difference 202: note = g or a
ttgatcagcgaagaaaggccaaccaataattggcactccagcgctcaaactctccagtgtcgagttccaaccacaatgcgataaaaaccctcctattgcagggtggttcaaaacctcttcttgtggacaccaactacaaatcacacatctttctcttgtctcttccacaaactcagttggaataattcccgagtttccatcnatgatgtcgggtcttataatccaaacaaagggtttcccactgttagccaaaccccaagcgaactcaacaagttcgtcaggtgtcatagccgtgatgctgccgaaattaacataaatcaccgaattggcctccctagaattcaaccattgcaagcattcaagctcttctttccatagattagatccaatggatgacaaactt

SEQ ID NO: 8 - DNA, 403 bp, Cannabis sativa
  source 1..403: mol_type = other DNA
  misc_difference 202: note = g or a
gagtgccgtatatttgtatttaaacattagtcaaccaatatgatcaaatgtatatatacggttacatattacgcatatatatgaatcaaagttatattactttctcaatatgatcaaagttgtgattttgttggtgctagccacactcattaatcaagtatggagtagtactacaagtaataattattgcatagagaagganagacaagctcttctcaacttgaagaaaggctttgtcgatgatggcaatcgtctatcctcatggacaagtagtagccgtgattgttgtgcatggagaggtatcaggtgcgataactcaaaaactcatcgtcatattatcgctcttgatcttaaatctgatgacaacaatcataattatttgggtggtgaaattggtccttct

SEQ ID NO: 9 - DNA, 403 bp, Cannabis sativa
  source 1..403: mol_type = other DNA
  misc_difference 202: note = g or a
gtcgcaaatggaaatttacgcccgcgatgtactcgaattaaacccattaaccccatttctcaggtactccacaaaatcatcatattactttttctttctaatttcacatttttttgaatttgtttttgggttttggtggaaataggtgaaaggattgccatttaatcggtattcatggctaacaacccacaatgcgtttgcnaagctgggacagaaatcgcagacgggaacaccgattgtgtcttccatgaatcaacaggactccattactagccagctcaatgtaagtttttttttttttctttttagttagtgaaatttatgttgtttgtttctcgggaaagttttgcggtaaaattaaggggaaaatatgatcaatgactggactttacaataactaaaa

SEQ ID NO: 10 - DNA, 403 bp, Cannabis sativa
  source 1..403: mol_type = other DNA
  misc_difference 202: note = a or c
aaatggcaccgcatccgaaacaacaaatatcccaatcaaacgaaagaactcacttattgccttatgacaccgtcttgcttcatccccactctgatcatcgctacccgatgctccgaaataacgctttccagccaccattctcaccaccatgttaagagttaaatcttctaaccaacgactcaactcaactacaactttacangacttgtagagctctctaatccctacttcaacctctgaaatcctcacttgcttcaacatctctagacggcggttagagaggagttctaacgtggcgatcttcctcatttcgcgccaaaaagggctataaggtgcgaaaccaaagactgcgtagttgtagcccatgtgcttggctgccacggttgtagggcgcgaggccagc

SEQ ID NO: 11 - DNA, 403 bp, Cannabis sativa
  source 1..403: mol_type = other DNA
  misc_difference 202: note = g or c
gataatttgatcttaccttgttgtgtaccataagtaatcagtggagtctctggtgacatttaactgctctaagagaccaactgctgtgatttttgtatcttcatccactgaagacatgtcttcactaaatgtctcccatgagtacaatgcacctttcaagggcgacatttgcgttcgagtcatttgtactctcacctgttangaagtgacagatgcccgttatagacaatgagtgaagtagccaatgcatcttacatatagtacacgatcaaccctgtgataaagcgggctagccctgatatggtattatgttcttatatcaagctcaataattaatcgttttcttatggcccatttagtctcagggtcagtcctgttatagtattatgttcttacacattat

SEQ ID NO: 12 - DNA, 403 bp, Cannabis sativa
  source 1..403: mol_type = other DNA
  misc_difference 202: note = a or g
cttcagcccaaaccttttgagcaacctcaaaagtcctatcaataccatgctcaccatcaagtaagatctctggctcaacaattgggaccaaaccattgtcctaaacataacaaaacataatcaattttcacaaacacaagtacaaaaatcaagattagcttagtcttgtctgattagttatcttacttgggagatagcagcntagcgggctagaccccaagcggcttccttcacagccagagctgatgggccattgggaatgctcacaacagtacgcctgtttaaataatgtaattttgtgtcaaataccgattaaagagcattgctataaggcattatagtgtccaacatcactattaggcactacgggtgctttttaacatttctcaaccagacaaaagta

SEQ ID NO: 13 - DNA, 403 bp, Cannabis sativa
  source 1..403: mol_type = other DNA
  misc_difference 202: note = c or a
tcgacttcaatgagaaatctcaacccctgtggtaagaattttgtgtacttttttaaaaattgaatttttaaaatttcccagttcgctgaatatttgtattaactgtgtctaatttttcatactagacattgaaagaatggtgtctttgaagggaatggttatccgttgcagttcaataattccggaaatcagagaagcaatntttagatgtcttgtgtgcggttactactctgaacctgttgctgtagagaaaggtaattagttaggataccattcgcatgggctagttttttcttatctatatcacaatgtcattctaaattattttcccttttcaggacggataactgaaccaacaagatgcttgaaggaagaatgccaagcaagaaactccatgacactt

SEQ ID NO: 14 - DNA, 403 bp, Cannabis sativa
  source 1..403: mol_type = other DNA
  misc_difference 202: note = c or t
ttaacttaatgatcatatagatagtaataaactattaattaattaattttgctggggatgagaatggtggccaggtagcttttccttgatcttttccataacagttttcttctcgtgaagaggagtaggatcatgagtagtagcagttggttcgtccttgtgttttccagtaagtttctcttttatttttgttcctaaaccnttcttcttccttctccctccagctccatcatcctctgactgcatccacacccattttataattattatatatattaatcattcatttttaatatatataattacattatattattattttaatattttaaaatgtaaataaaaaatttagtgattcaaattataattttttatattatatgttgatagcaattttgttatt

SEQ ID NO: 15 - DNA, 403 bp, Cannabis sativa
  source 1..403: mol_type = other DNA
  misc_difference 202: note = a or g
cgatcacttcgtagatgcatcctcccacaaggtagcacaattgtagaaagtgctaaatcatgctttattccatttgttctttttgtcttctctttttgcttaatcgaacgatgttgtgaacttgtagggttgtcaaattcgaagggagagcgcacatggagagagcgtttgttgcaacaatgtgagggccctgtttgatganctcccaactccacacctaattgtggagatcacaccattccctgaagggcctctcactgaaaaagattacaccaaagctgagaaattggagagggtacttagaactggcccgaacgtttgattcttctctcgagttaaatcatcgctgtctctcgttagaactacagcttaattgtatgtatgttttgagccttgtacatat

SEQ ID NO: 16 - DNA, 403 bp, Cannabis sativa
  source 1..403: mol_type = other DNA
  misc_difference 202: note = a or t
ccatataatgcaaattctctaatatcaattacaaaatcaaatataagacacagatgcctaaatgatgccatgtaatttcgatcacagcattgaatttttcttcaagattgaagagttaagaagtaaagaactttaccatagatgtgtaggagacattgtaacgaaaaagaggcgcttcttgcgaggatcaactttagtagcnacccaatctgcccaagcttccatagccaactccatggcaccaacaccatctaactcttcacaaattccactttcatcagaactccatctgcataataaattggttgcatagtaagtacaaagtgctcaacacatcatattctgaaacttaactcaaaatgttactatgattcattttacttacaacagcttaactggacct

SEQ ID NO: 17 - DNA, 403 bp, Cannabis sativa
  source 1..403: mol_type = other DNA
  misc_difference 202: note = a or g
gaagtatgggaaagaaagagcttgagtcaagaagctttgattgaggcattcaaaatggaagtgaatcaagtaaaaatggagttaagaagtgcaaatgagttggtgagccagttgatggatgatgttgaaatacttacttatgatatactaaaggcgaaaactgagattaatgaaatgaagaggaaagaaacagaagctcaanttgagatagcactaatgaaaactgagcttcagaaagagagaagagattgcaatgatggtcacataacggcttcactcaaggaatacgagtccttggtcaaaaagggtgatgatcaaatttgggcgccaacttctgataacaagcatgagctggaaactttgaggaaggagttggatgctgcattggctaaagttgctgaat

SEQ ID NO: 18 - DNA, 403 bp, Cannabis sativa
  source 1..403: mol_type = other DNA
  misc_difference 202: note = t or a
tttgttaattgtttcatttttctgaaatgcaaatgatgatatttttatgaaggttggaagaatggcgggtcagtttgccaagcccagatcagattcatttgaggatagggatggagtgaagcttcctagttacagaggtgacaatgtgaatggtgatgcatttgatgaaaaatcgagaattccagaccctaatcgaatgatnagagcctatacacaatctgctgcaactttgaatcttttgagggcatttgctactggaggttatgctgctatgcagagagtgacccactggaatctagatttcactgatcacagtgagcagggggataggtaaacttttattgttcttttcttcttacttgaaattttgaatgtttattttccataatgaataggattgaag

SEQ ID NO: 19 - DNA, 403 bp, Cannabis sativa
  source 1..403: mol_type = other DNA
  misc_difference 202: note = a or g
cactttaagttataaattacgttgtaactaaaagtaaaaatctttgtagtgtaaatttatatatatttacctcggagaccatgtcattgaaaactcttcccattatttccatcttatgtttaggatcattactcataacacttcccatcatggttagtaccatactcccaccacttactatttcctctgaacgacatcttanaaagcttgtgaaatcctcttgaaattgattcaagtaagctttcacaactgaattagggcttctttttgtaatataaatgttgcctttgttaagtgcttctcctgtttccccaaccaaaccacttggaaccttcaaaacaacattaattcacataattaataaactaaaaccttataaaaagaaagggtaaatttcaatttt

SEQ ID NO: 20 - DNA, 403 bp, Cannabis sativa
  source 1..403: mol_type = other DNA
  misc_difference 202: note = c or a
acaagtcttatctaaacgaaaagctccacaactatggagatgaacactaccagagaagcaaaacataacagttagcttcagttcataatttatttacagtctatcatacactgttctacaagtgcttgctgaggatggattcgacaacaccttgtgcaataggatgataacaatcgcgagcctctgcaaacacccttttcgngaaaatcttctcttcttccttcccgttcccttgaacgagcgcagtgtagagtggacgaaggtatttcatcctcccaacttctttgagagttttctccacctcgccgtagtagtctctgcacttggcggtaatggctagctgcagaaaccccaccttcacctcgtaatcttttgattctgagagcctgtagcgctggtccaa

SEQ ID NO: 21 - DNA, 403 bp, Cannabis sativa
  source 1..403: mol_type = other DNA
  misc_difference 202: note = t or c
ctgtctcatatattcaacctcgtcacaaacatttattgtttcgtgggtcgttttccacttcttcatatctacacaataatcaatcggggcttcctttgttttttgttatgtgaaaatggttgcagaagtagaagaagaatgagagagttaagattagaaagaaagaagaatgcgaaccctttgtgacgtttgtgagaaagcngccgcgatcctcttctgcgccgccgacgaggctgctctatgcagttcctgcgatgacaaggtctttcactaatttaagcttttaataatttttaatttaatttactaacttcaaatcattggtatgaagttatttcaattcaaatattatttatatgaattgggttttgtattttgttatctgctggaattttcattgttt

SEQ ID NO: 22 - DNA, 403 bp, Cannabis sativa
  source 1..403: mol_type = other DNA
  misc_difference 202: note = a or c
agcagagaaaattgaaaaaatgacggaaaggaaaaagaagagggaagcaacaccagtatgctttgtggccacagggaagtgaacaaggtataaatccagataatctaactgaagcttcttcagactattcttacaggcctcaaggacatgtccatggtcagaattccaaagctgcaatcaagacaggtaaagaaaaagaatnatagtctggctggacatacagaatatacatattattcgatcagttcgaaaataaactaaccttagtggtaacaaagagatcttctctcttaacaagccctgtctgaaatgcctcagaaagtgcctccccaacttctgtttcattcctgtaatcagctgcaaccaacaccagctagtcaaacattacattgtagcaactcaa

SEQ ID NO: 23 - DNA, 403 bp, Cannabis sativa
  source 1..403: mol_type = other DNA
  misc_difference 202: note = a or c
ataatgggttttgatttgttctgatactcagattgaaccaaagagaaggccatctccgagttaccttgagaaagttcagagtgaaatcagtgccaacatgagaggagtattggtggattggttggtggaagttgcagaggagtacaaacttggttcagagactctttacctatctgtttcttatattgatcgattcttgtcnttgaacaccattgccaggaataagcttcagttattgggtgtttcttctttgctcgttgcctcgtaagattctaacccttttgaactaatgttaatgaagatgatatgttgaatttgatttgtttattcatataaagttttgatttttatctttggtttcacttgtttagaaagtatgaagagattaatcctcctaatgtgg

SEQ ID NO: 24 - DNA, 403 bp, Cannabis sativa
  source 1..403: mol_type = other DNA
  misc_difference 202: note = c or t
gacctggcgccggatcgatccggttccgaaatacgcggacgggttgcctatgttctgccacgtggcgggttgtgaaggtaagctggttgtaatgggcggttgggatccgaaaagctacggacccgtttcggatgttttcgttttcgatttcgctaagaatcggtggagcgaagccaaggccatgccgggtaagaggtcgttntttgcggtcgggtcttattcgggtcggatctatgttgcgggtgggcacgatgagaataagaacgcgttgagttctgcttgggtttacgatgtgagtctggacgagtggagcgagttggctcagatgagtcagggccgtgacgagtgcgaaggcgtggtgttaaacggtgagttttgggttgtgagtgggtacggcaccgac

SEQ ID NO: 25 - DNA, 403 bp, Cannabis sativa
  source 1..403: mol_type = other DNA
  misc_difference 202: note = a or g
atagtgagttctttaatgaaacgtacccattccattatttggtttagtttcatttaatattattgaaagtggaacatcaaatttaagaagatccatattttaattggttttggaaattgtacatgaggaatgattaagaagacgtaggcaaccaattatagatgcacattaatttagttcatttagatcctaatccatgcanctggcccaattattggagatctcatggttttgggtatctcaaatctaagcatagccactggctcagtcggagatctcatggttcttgcaatatctttgagaataaatttctgaattttcccagttgaggtcttgggaagttcatctctgaacacaacaactttaggaaccatataatgaggcaacttctctctacaatact

SEQ ID NO: 26 - DNA, 403 bp, Cannabis sativa
  source 1..403: mol_type = other DNA
  misc_difference 202: note = a or g
aggttacatcatcaataaatgaatagcactaccaatagaaggaaagagtgatcatgtgacatatttcatatatggctcttgaaactaaccaaacaccactcaaaaaaaatctacatttctctccacatatcacaccttcccctactcatattttcttctatatatgtcactaaatcataaactcccacatacaagtactctnctcaagccttattacaaacactcctctcttctacaaaaacctctctgtttcaaacatggctataagattacatggaatgttgaatgctaagcacctcctacgccgctcgagtttctctcaaaaccatggagtttcaacacccatcgacgttcccaaaggctatctcgcggtgtatgttggagaagaacacaagaagagaca

SEQ ID NO: 27 - DNA, 403 bp, Cannabis sativa
  source 1..403: mol_type = other DNA
  misc_difference 202: note = t or c
cctattcaagatatcatggccttcagttcggcaattcataattcttcgaaagacttctcttagttgacgctccaatggatccaatccatttcgaaaactttcacaaaccgcagctacctcatgcaccctttgctctacctcctccttttgctcttctgtcaagggaaactgaacagaatccaccaagtcagtcatgtggcgngcacatctttcaacctgataaatctccctcaacaatccattggtgtttcgattctctctctttttggattcttccaaaatccgattgagaagtgacatcaaaggcgttccccatgagaaattgcggggcactgagaaatggacattaagaccgcgatcctgacatgggattgcagccacaagagtccacaaaacaaacatg

SEQ ID NO: 28 - DNA, 403 bp, Cannabis sativa
  source 1..403: mol_type = other DNA
  misc_difference 202: note = g or a
tggtcagtgtgaattccactattgtgtgaattgtgtcacttgcacatccaggcaaagagtaaaatatgaatatcatgatcatcctcttttttttctcgacaaaattcacactggtctggtggaatgtgatgattatgattcttattgtaagaatccaattttggctgaatgtgacgaattcaagtataccaattcatctgtntttggttgtgatcaagaatgtaattttaaagttcatttgctatgtggtccattacctagcactcttaaatatgaatatcacatacatcctcttattttgtttgattttgttctcaacaatgattatggagacttttactgtgatatttgtgaaatgaaaagagatccacgaatacgtgtatacttttgtgaagattgcaac

SEQ ID NO: 29 - DNA, 403 bp, Cannabis sativa; identical to SEQ ID NO: 14
  source 1..403: mol_type = other DNA
  misc_difference 202: note = c or t

SEQ ID NO: 30 - DNA, 403 bp, Cannabis sativa
  source 1..403: mol_type = other DNA
  misc_difference 202: note = c or a
aagacaaaagaataagctaattaagaagctaaagaaagagatcaactctactttgaatttgttgtttcagaacaatatgactacaaatcgatggtgggctggtaatgtggccatgagaggagtggattccatgtcttcaccaccaccaccacctccattgcttcagctcagaaacacagatgaagatcccaacaaagatgangaccaagacagtggcaacggcggcgacgacgacgaccccaattcaaccggccacgaaagcttcggactaggaggaagcagcagcaacaaccgcaggccacgtggcagacccccaggttccaagaacaagccaaagcctccggtagtgataacaaaagagagccccaatgctctaagaagccacgttttggaaatcagtagc

SEQ ID NO: 31 - DNA, 403 bp, Cannabis sativa
  source 1..403: mol_type = other DNA
  misc_difference 202: note = a or g
tatctccaaatacccatctgggtgcataactcccacatcgcccgtgtaaaaccatccatcctcctttatagctttagcagtagccagttcatctttaaggtagcccaacatgacgcaattacctcttaacaccacttcccccattgtgaaaccatctctcttcacactcaaccccgaattcggatcaacaacatcaacttcngtcattgctgctgaattcactccttggcgcgcctttagccgggcgcgctcagtagctggaagaagattccacttcttcttccaagcgcaagacacaacgagtccgcacacttctgtcaacccgtagccatgactaactttaaaaccgagcgactcagttcggtgtagaaccgccgcgggaggtggagctcctgcggtaagg

SEQ ID NO: 32 - DNA, 403 bp, Cannabis sativa
  source 1..403: mol_type = other DNA
  misc_difference 202: note = g or a
tgtttatattatgaggaaaagaggtttggtcccaagcttggtgtcatataattctattgtacatggacttagcaaggaaggaggctgtatgagggcttatcagttgttagaagaaggcattgaattcggatatcgaccatctgagcatacttacaaggtcttagtagaaggtctttgccgagaaaatgaccttcacaaggcnaaatttgtctttcatgttatgctcaacaaggaaggagttgagagaactagattttataacatatatcttagagctctttgttttatggataatgctgctactgagcttttgaatacacttgtttctatgctccaaactcagtgtcaacctgatgtgatcactctcaataccattattaaagggttttgtaagatggggaga

SEQ ID NO: 33 - DNA, 403 bp, Cannabis sativa
  source 1..403: mol_type = other DNA
  misc_difference 202: note = c or g
tcaattttgttactgttatctttaatgctttcaattgaataaaattcaaggggttaatattatagcaattctgcagcgtctctacatattgattatcctatgctgtttaagcttcagaatgactctgcaaataaagtctcacactgtggtgtcttggagtttattgctgaggaaggcatgatatatatgccttattgggtanggattggttttttctgcgaaatattcacctgtacagagttttctggtccatttttatctagcttttgacccatggtgatttgcactcattagtgttttgttggttctgtttatctattagatgatgcaaaatatgctcttaggagagggagactttgtgcgtgtgaagaatgtgactcttctgaaggggacatatgttaaa

SEQ ID NO: 34 - DNA, 403 bp, Cannabis sativa
  source 1..403: mol_type = other DNA
  misc_difference 202: note = g or a
attcatgaaagaaaatttctaataatattcatttgtatcattcttggggactacatttagttgttaatccccaaggaaaaattaattacaaggtatatggcattctctgcctaacaaattgtaactgtgagttttgttagctgttatcttctactggtgtctagactcatttgattaggagcttaagtgagaaataagatcnatgaaggcatcttctgtgcatggaattgtaagagctcccattggatgatcgaacccgaattcttcttcagcttgatttagaaagtcttggaatgaaagctggttcaagtatgccacagggatcacaaatctcttcattatgctctcatcgcgaacataaaccgccaaaaagcctttagggatatcttttgtgcttgagaaa

SEQ ID NO: 35 - DNA, 403 bp, Cannabis sativa
  source 1..403: mol_type = other DNA
  misc_difference 202: note = c or a
cacagcatctccattagaaggattaaacttatgagtactagcatgagcatacccacttgcaaacctaaaaagaccacttccaccaatcacaggcatttctctaaccttatcaaacacttggtttctaccaagaatggtgagagtgctcccattgtatttcccttgagttatatgaaaattcatagccataattagggcaatntcttcttgtgaagctaatccataaaacccttgagcttttcctagcaactttgagcttacttctggcccttctgtcaatggattgtcgatcatgctaaccgccccgaacccacttttcgatgcattggccggtggttggattattgccatcgcgctagggtttttgccgctgtatatgtcgtgccaatagaaccgaaagtgg

SEQ ID NO: 36 - DNA, 403 bp, Cannabis sativa
  source 1..403: mol_type = other DNA
  misc_difference 202: note = c or t
acgtaatgctttgatgtattttctgtcatctaaattcggaataccatctagaatattttgacagtcttccacattacatgaactagtgatattgcttgattttccattgattatcttctcccttgtatcaatatacttacccatactttcactttgtacaagtgatgcctcagttttagcagctatactttttgcaagttcntccatcacatgttcaatgatagaattagtccctttgttctttttctcctttggtcttggtgcatcaagtggaacagaagtagctcgacgacgtacaccagtaacttcttcttcctctccaaaattttcaccatcgcattcaataccatcatcaacatgtgctccagtgtcaatataatgccgctcttgttcatgttcctca

SEQ ID NO: 37 - DNA, 403 bp, Cannabis sativa
  source 1..403: mol_type = other DNA
  misc_difference 202: note = a or g
agtagtagcaacggtgaattgacaacaaaagtttgaagtcattaactaccccatggccatcaaaaactcaccctcacatacaacctcacctccaacatatgctttcccctccattttcgctattccaaagcgtttctgcagctttgtgagtgtcattctcataactaaagtgtcaccagcaatcactggtttccggaatctnactttgtcgattccagcaaaaaagaagttctcccgagaacctcccacttctggttgcagcatcaccaaaccaccaacctgagccattgcctagatccaacaaaattgaatatgttgttttgttaaaattacattacatgatgactaaatagatcagaatatttcctacaaggacagcaaaaaaattcatttttctaggaac

SEQ ID NO: 38 - DNA, 403 bp, Cannabis sativa
  source 1..403: mol_type = other DNA
  misc_difference 202: note = t or c
tttatattacctacatgaaatcagacaacaacagcatatcgggggcttcactcatggagatggagacctactaccatttggttcatctgggtagcgatttggacttcggctaaggcttctctttgggcttcggcttctgcttcgactatggcttggacttctagctccacttggacccctagctgggcttcgacttgggctncttgatccattatatggtggagaacgagagtacgacctgtctcggctgtaccttctctcctgtggagacacagatctggcccgaagagggagtacgaaaattaatgtttaaacattgaaatatattaaatgcatgttttctcctattgctaaagatcccacatttttaatgctgactagagaagttgaaaagatatacttg

SEQ ID NO: 39 - DNA, 403 bp, Cannabis sativa
  source 1..403: mol_type = other DNA
  misc_difference 202: note = t or a
caaaattattttagtaaaggttccttctttagataagaaagaacaaaatatggccattgtatcatcaaccatttcaaataggaaacaattagattctcagtactctaaacctttgattcctgatcctgctagtaccaaacacatggctgctcttaagcttcaaaaggtctacaaaagcttccgaactagacgaaagttagcngattgtgctgttcttgtagagcagagttggtatgtaactaatcttttatgttcaattctagtctttgttgtgttgtgttgttctgtcatgttctgatatacaattttttgcatcaattttaggtggaagctcttagattttgctgaactcaagaggagttcgatatcttttttcgatattgagaagcatgaaaccgcgatt

SEQ ID NO: 40 - DNA, 403 bp, Cannabis sativa
  source 1..403: mol_type = other DNA
  misc_difference 202: note = a or g
aagtaaataatatttacataagtgaattagaatgaagtaataagcaataaagtgccacaaactccaacaaatccaactacaaatcccactccaattcccatataaagccatgacatgttaagccacccttcatcatattcttcagcatcacttggatcatgaatatctttagggctatttggtgtctcatctccaagacatnaattgtttagtggaagtccgcacaatccatcattatcaatgtatatcgaagcattaaaactttgcaattgagtaccgataggaattcttccagacaatttgttacttgacaaattcaaaaaagatagagaagatatacttgccaagcttgttggaatgacactagaaagcttgttatgggataaatctagagaatccaact

SEQ ID NO: 41 - DNA, 403 bp, Cannabis sativa
  source 1..403: mol_type = other DNA
  misc_difference 202: note = c or t
ttttatttctctcactttttttgcatactcttttctttccattcttctcgatcgtgctgaaatacctaataagacagtgacacagcatggcatgacacaattaatgggagcggttgcctttgcacaacaactcctcctcttccacctccactctgctgatcatatgggaccagagggacaatatcacttgctactccagctngtgattcttgtctctctggtcacatctctaatgggaatagggctaccgaagagtttcttagtgagttttgttaggtctcttagcattttgtttcaaggggtttggcttatggtgatggggtttatgttatggacaccatccttgatttccaaagggtgtttcatgcactatgaggaaggtcatcatgtggtgagatgctca

SEQ ID NO: 42 - DNA, 403 bp, Cannabis sativa
  source 1..403: mol_type = other DNA
  misc_difference 202: note = t or g
gaatgtgttggaatcccattcttaatttgatattgaaatttttattgctgttttccagtcttggcatgaaagactcttgggggcctttgaaggctttggctgtagctagcattataaatggcattggtgatatactcctgtgcagagtttttagctatggcattgctggtgcagcatgggcgacgatggcatcacaggtgcnccagatgaacattttgctccactgtctttccagattatatatttcactttagttcttattaatttccgagaaatatcctaaatgagtttgttttctttcatactgccactaacacaaatagtattaggttgttgcagggtatatgatggttgaaaatctgaacaagaaaggttacaatgcttatgctctctccattccctc

SEQ ID NO: 43 - DNA, 403 bp, Cannabis sativa
  source 1..403: mol_type = other DNA
  misc_difference 202: note = t or a
gacgatggcatcacaggtgctccagatgaacattttgctccactgtctttccagattatatatttcactttagttcttattaatttccgagaaatatcctaaatgagtttgttttctttcatactgccactaacacaaatagtattaggttgttgcagggtatatgatggttgaaaatctgaacaagaaaggttacaatgcntatgctctctccattccctcaccgaaagaacttatcgctatacttgagcttgctgctccggtattcatcactatgacttctaaggtaaatattactcagttttccttgagcttggctataatctttccttagttttccttcaaaaactaaggtgtttatatccttaggtggcattctatagtctcctcatatattttgcta

SEQ ID NO: 44 - DNA, 403 bp, Cannabis sativa
  source 1..403: mol_type = other DNA
  misc_difference 202: note = t or g
taggcaaagcatgtcaagactggggcttctttatggtaattattaattaagcttaattaatattaatacctctaaaacaatattgatgtcatatttgaaatattgctattcttttatgattaatatataggtgatcaatcatggtgtggcagagagattaatgagtgaagttttagaagggtgtagaggtttttttgatctnagtgaagaagagaagcttgtgtttaagggtacacatgttatggacacaattaggtatggtacaagcttcaatgcatcggtagagaaagctttgtattggagagattatcttaaggttcttgttcctcagcaccatcctcatcattttcatttccctaataacccatctgggttcaggtaatattattcacacataaaattt

SEQ ID NO: 45 - DNA, 403 bp, Cannabis sativa
  source 1..403: mol_type = other DNA
  misc_difference 202: note = c or t
cgacttctttgccgaagataagaaaaaccctgatagcagtttcgatgaatatttctacgatgatgacgaaaagcctcgcgaggagtgtggtgttgtgggtatttatggcgactcagaggcctctcggctctgttacttggctcttcacgctctccaacatcgtggtcaagaaggggctggaattgttgctgtgaaaaacgangttcttcaatccgttacaggcgttggacttgtctctgaagtctttagccattcaaagctcgatcaattgcctggagatttggctattggccatgtacggtactctactgctgggtcttctatgcttaaaaatgttcaaccttttgttgcagggtatagatttggttcagttggtgttgcacacaatggcaatttggtaaat

SEQ ID NO: 46 - DNA, 403 bp, Cannabis sativa
  source 1..403: mol_type = other DNA
  misc_difference 202: note = g or a
gatgaacattttgctccactgtctttccagattatatatttcactttagttcttattaatttccgagaaatatcctaaatgagtttgttttctttcatactgccactaacacaaatagtattaggttgttgcagggtatatgatggttgaaaatctgaacaagaaaggttacaatgcttatgctctctccattccctcaccnaaagaacttatcgctatacttgagcttgctgctccggtattcatcactatgacttctaaggtaaatattactcagttttccttgagcttggctataatctttccttagttttccttcaaaaactaaggtgtttatatccttaggtggcattctatagtctcctcatatattttgctacatccatgggcacaatcagcatgg

SEQ ID NO: 47 - DNA, 403 bp, Cannabis sativa
  source 1..403: mol_type = other DNA
  misc_difference 202: note = c or t
tagtgggttaggaactgggagcaaaggcctaagcggcggacagaagaggagagtgagcatctgcatagagattctcactcgtccaaagcttcttttccttgacgagcccactagtgggctcgacagtgctgcttcatactatgtgatgagcagcattgcgtcgttggatattcagagtcggaggggtggtggggccggtggncggaggactgtggtggcttccatccaccagcccagttccgaagtgtttcagctttttaatactctttgccttctttctgctggtaaaattgtgtattttggtcctgctagtgcagctaatgaggtattttcaggttttttttaaacgtatttaaattataaattacaatacataaaggaataataatatttgtactaatta

SEQ ID NO: 48 - DNA, 20 bp, synthetic construct
  source 1..20: mol_type = other DNA
gcaaatgagttggtgagcca

SEQ ID NO: 49 - DNA, 20 bp, synthetic construct
  source 1..20: mol_type = other DNA
gccaatgcagcatccaactc

SEQ ID NO: 50 - DNA, 20 bp, synthetic construct
  source 1..20: mol_type = other DNA
ggggtgatgtgaatgagtgc

SEQ ID NO: 51 - DNA, 20 bp, synthetic construct
  source 1..20: mol_type = other DNA
cctgggatgctgtgtgttct

SEQ ID NO: 52 - DNA, 23 bp, synthetic construct
  source 1..23: mol_type = other DNA
aaattaagagcagtttcacgtgt

SEQ ID NO: 53 - DNA, 21 bp, synthetic construct
  source 1..21: mol_type = other DNA
tgctcgaatctgctacattgc

SEQ ID NO: 54 - DNA, 20 bp, synthetic construct
  source 1..20: mol_type = other DNA
aagactcttgggggcctttg

SEQ ID NO: 55 - DNA, 23 bp, synthetic construct
  source 1..23: mol_type = other DNA
ccatcatataccctgcaacaacc

SEQ ID NO: 56 - DNA, 20 bp, synthetic construct
  source 1..20: mol_type = other DNA
ttacccatcgtggctgactg

SEQ ID NO: 57 - DNA, 20 bp, synthetic construct
  source 1..20: mol_type = other DNA
cgcaactaacggcaaccaaa

SEQ ID NO: 58 - DNA, 20 bp, synthetic construct
  source 1..20: mol_type = other DNA
ttcacaaaccgcagctacct

SEQ ID NO: 59 - DNA, 20 bp, synthetic construct
  source 1..20: mol_type = other DNA
tggactcttgtggctgcaat

SEQ ID NO: 60 - DNA, 21 bp, synthetic construct
  source 1..21: mol_type = other DNA
agcaacggtgaattgacaaca

SEQ ID NO: 61 - DNA, 20 bp, synthetic construct
  source 1..20: mol_type = other DNA
aatggctcaggttggtggtt

SEQ ID NO: 62 - DNA, 20 bp, synthetic construct
  source 1..20: mol_type = other DNA
ttggaagaatggcgggtcag

SEQ ID NO: 63 - DNA, 20 bp, synthetic construct
  source 1..20: mol_type = other DNA
tatccccctgctcactgtga

SEQ ID NO: 64 - DNA, 20 bp, synthetic construct
  source 1..20: mol_type = other DNA
acaccgtcttgcttcatccc

SEQ ID NO: 65 - DNA, 20 bp, synthetic construct
  source 1..20: mol_type = other DNA
tgggctacaactacgcagtc

SEQ ID NO: 66 - DNA, 20 bp, synthetic construct
  source 1..20: mol_type = other DNA
tgttggtgctagccacactc

SEQ ID NO: 67 - DNA, 20 bp, synthetic construct
  source 1..20: mol_type = other DNA
aggaccaatttcaccaccca

SEQ ID NO: 68 - DNA, 20 bp, synthetic construct
  source 1..20: mol_type = other DNA
aagcatgtcaagactggggc

SEQ ID NO: 69 - DNA, 20 bp, synthetic construct
  source 1..20: mol_type = other DNA
accgatgcattgaagcttgt

SEQ ID NO: 70 - DNA, 20 bp, synthetic construct
  source 1..20: mol_type = other DNA
gaaggaggctgtatgagggc

SEQ ID NO: 71 - DNA, 21 bp, synthetic construct
  source 1..21: mol_type = other DNA
ggttgacactgagtttggagc

SEQ ID NO: 72 - DNA, 20 bp, synthetic construct
  source 1..20: mol_type = other DNA
aagggtaagggagagtggca

SEQ ID NO: 73 - DNA, 20 bp, synthetic construct
  source 1..20: mol_type = other DNA
agctcgactaatggggatga

SEQ ID NO: 74 - DNA, 23 bp, synthetic construct
  source 1..23: mol_type = other DNA
gcattctctgcctaacaaattgt

SEQ ID NO: 75 - DNA, 20 bp, synthetic construct
  source 1..20: mol_type = other DNA
cctaaaggctttttggcggt

SEQ ID NO: 76 - DNA, 22 bp, synthetic construct
  source 1..22: mol_type = other DNA
gtgatgcctcagttttagcagc

SEQ ID NO: 77 - DNA, 20 bp, synthetic construct
  source 1..20: mol_type = other DNA
ggaacatgaacaagagcggc

SEQ ID NO: 78 - DNA, 21 bp, synthetic construct
  source 1..21: mol_type = other DNA
tccacatatcacaccttcccc

SEQ ID NO: 79 - DNA, 20 bp, synthetic construct
  source 1..20: mol_type = other DNA
tctccaacatacaccgcgag

SEQ ID NO: 80 - DNA, 20 bp, synthetic construct
  source 1..20: mol_type = other DNA
ttatggcgactcagaggcct

SEQ ID NO: 81 - DNA, 21 bp, synthetic construct
  source 1..21: mol_type = other DNA
accaaattgccattgtgtgca

SEQ ID NO: 82 - DNA, 20 bp, synthetic construct
  source 1..20: mol_type = other DNA
tattttccggcgttcctccg

SEQ ID NO: 83 - DNA, 20 bp, synthetic construct
  source 1..20: mol_type = other DNA
ggagagactcaaggcggttc

SEQ ID NO: 84 - DNA, 21 bp, synthetic construct
  source 1..21: mol_type = other DNA
caattctgcagcgtctctaca

SEQ ID NO: 85 - DNA, 20 bp, synthetic construct
  source 1..20: mol_type = other DNA
cacgcacaaagtctccctct

SEQ ID NO: 86 - DNA, 20 bp, synthetic construct
  source 1..20: mol_type = other DNA
tgtttcgtgggtcgttttcc

SEQ ID NO: 87 - DNA, 20 bp, synthetic construct
  source 1..20: mol_type = other DNA
accttgtcatcgcaggaact

SEQ ID NO: 88 - DNA, 23 bp, synthetic construct
  source 1..23: mol_type = other DNA
cagataggacactctgaacaagc

SEQ ID NO: 89 - DNA, 20 bp, synthetic construct
  source 1..20: mol_type = other DNA
tcaggctcgaatgcaagaga

SEQ ID NO: 90 - DNA, 22 bp, synthetic construct
  source 1..22: mol_type = other DNA
gagaaatctcaacccctgtggt

SEQ ID NO: 91 - DNA, 20 bp, synthetic construct
  source 1..20: mol_type = other DNA
aactagcccatgcgaatggt

SEQ ID NO: 92 - DNA, 21 bp, synthetic construct
  source 1..21: mol_type = other DNA
tcagtggagtctctggtgaca

SEQ ID NO: 93 - DNA, 20 bp, synthetic construct
  source 1..20: mol_type = other DNA
ggctagcccgctttatcaca

SEQ ID NO: 94 - DNA, 20 bp, synthetic construct
  source 1..20: mol_type = other DNA
tgctggggatgagaatggtg

SEQ ID NO: 95 - DNA, 20 bp, synthetic construct
  source 1..20: mol_type = other DNA
atgggtgtggatgcagtcag

SEQ ID NO: 96 - DNA, 20 bp, synthetic construct
  source 1..20: mol_type = other DNA
agagaaggccatctccgagt

SEQ ID NO: 97 - DNA, 21 bp, synthetic construct
  source 1..21: mol_type = other DNA
gaatcttacgaggcaacgagc

SEQ ID NO: 98 - DNA, 21 bp, synthetic construct
  source 1..21: mol_type = other DNA
acctcggagaccatgtcattg

SEQ ID NO: 99 - DNA, 20 bp, synthetic construct
  source 1..20: mol_type = other DNA
ggtttggttggggaaacagg

SEQ ID NO: 100 - DNA, 20 bp, synthetic construct
  source 1..20: mol_type = other DNA
gcatgagcatacccacttgc

SEQ ID NO: 101 - DNA, 20 bp, synthetic construct
  source 1..20: mol_type = other DNA
cggccaatgcatcgaaaagt

SEQ ID NO: 102 - DNA, 20 bp, synthetic construct
  source 1..20: mol_type = other DNA
ttaatgggagcggttgcctt

SEQ ID NO: 103 - DNA, 21 bp, synthetic construct
  source 1..21: mol_type = other DNA
gcatctcaccacatgatgacc

SEQ ID NO: 104 - DNA, 20 bp, synthetic construct
  source 1..20: mol_type = other DNA
gatgcatcctcccacaaggt

SEQ ID NO: 105 - DNA, 20 bp, synthetic construct
  source 1..20: mol_type = other DNA
cgggccagttctaagtaccc

SEQ ID NO: 106 - DNA, 20 bp, synthetic construct
  source 1..20: mol_type = other DNA
ggacgggttgcctatgttct

SEQ ID NO: 107 - DNA, 20 bp, synthetic construct
  source 1..20: mol_type = other DNA
actcatctgagccaactcgc

SEQ ID NO: 108 - DNA, 20 bp, synthetic construct
  source 1..20: mol_type = other DNA
caagtgcttgctgaggatgg

SEQ ID NO: 109 - DNA, 20 bp, synthetic construct
  source 1..20: mol_type = other DNA
gcgctacaggctctcagaat

SEQ ID NO: 110 - DNA, 20 bp, synthetic construct
  source 1..20: mol_type = other DNA
caacagcatatcgggggctt

SEQ ID NO: 111 - DNA, 20 bp, synthetic construct
  source 1..20: mol_type = other DNA
ctcttcgggccagatctgtg

SEQ ID NO: 112 - DNA, 22 bp, synthetic construct
  source 1..22: mol_type = other DNA
acaaatcccactccaattccca

SEQ ID NO: 113 - DNA, 21 bp, synthetic construct
  source 1..21: mol_type = other DNA
gtcattccaacaagcttggca

SEQ ID NO: 114 - DNA, 21 bp, synthetic construct
  source 1..21: mol_type = other DNA
acgaaaaagaggcgcttcttg

SEQ ID NO: 115 - DNA, 24 bp, synthetic construct
  source 1..24: mol_type = other DNA
aggtccagttaagctgttgtaagt

SEQ ID NO: 116 - DNA, 20 bp, synthetic construct
  source 1..20: mol_type = other DNA
attgatggtggtgatgggca

SEQ ID NO: 117 - DNA, 21 bp, synthetic construct
  source 1..21: mol_type = other DNA
tccgaggacgactttctaggt

SEQ ID NO: 118 - DNA, 20 bp, synthetic construct
  source 1..20: mol_type = other DNA
cctgatcatgggcagcatca

SEQ ID NO: 119 - DNA, 20 bp, synthetic construct
  source 1..20: mol_type = other DNA
ttagaaacagcctggtgggg

SEQ ID NO: 120 - DNA, 20 bp, synthetic construct
  source 1..20: mol_type = other DNA
gcatcacaggtgctccagat

SEQ ID NO: 121 - DNA, 21 bp, synthetic construct
  source 1..21: mol_type = other DNA
agtgatgaataccggagcagc

SEQ ID NO: 122 - DNA, 20 bp, synthetic construct
  source 1..20: mol_type = other DNA
gtgggttaggaactgggagc

SEQ ID NO: 123 - DNA, 20 bp, synthetic construct
  source 1..20: mol_type = other DNA
aaacacttcggaactgggct

SEQ ID NO: 124 - DNA, 20 bp, synthetic construct
  source 1..20: mol_type = other DNA
acatcgcccgtgtaaaacca

SEQ ID NO: 125 - DNA, 20 bp, synthetic construct
  source 1..20: mol_type = other DNA
tcatggctacgggttgacag

SEQ ID NO: 126 - DNA, 20 bp, synthetic construct
  source 1..20: mol_type = other DNA
accaaacacatggctgctct

SEQ ID NO: 127 - DNA, 20 bp, synthetic construct
  source 1..20: mol_type = other DNA
atcgcggtttcatgcttctc

SEQ ID NO: 128 - DNA, 20 bp, synthetic construct
  source 1..20: mol_type = other DNA
gggaagcaacaccagtatgc

SEQ ID NO: 129 - DNA, 20 bp, synthetic construct
  source 1..20: mol_type = other DNA
aacagaagttggggaggcac

SEQ ID NO: 130 - DNA, 20 bp, synthetic construct
  source 1..20: mol_type = other DNA
cagcccaaaccttttgagca

SEQ ID NO: 131 - DNA, 20 bp, synthetic construct
  source 1..20: mol_type = other DNA
caggcgtactgttgtgagca

SEQ ID NO: 132 - DNA, 22 bp, synthetic construct
  source 1..22: mol_type = other DNA
acaaactgctgcctctgtatct

SEQ ID NO: 133 - DNA, 20 bp, synthetic construct
  source 1..20: mol_type = other DNA
gtgccagccattctcaaagc

SEQ ID NO: 134 - DNA, 20 bp, synthetic construct
  source 1..20: mol_type = other DNA
tgctccactgtctttccaga

SEQ ID NO: 135 - DNA, 21 bp, synthetic construct
  source 1..21: mol_type = other DNA
gccaagctcaaggaaaactga

SEQ ID NO: 136 - DNA, 20 bp, synthetic construct
  source 1..20: mol_type = other DNA
tcgatggtgggctggtaatg

SEQ ID NO: 137 - DNA, 21 bp, synthetic construct
  source 1..21: mol_type = other DNA
tggcttcttagagcattgggg

SEQ ID NO: 138 - DNA, 20 bp, synthetic construct
  source 1..20: mol_type = other DNA
tgatcagcgaagaaaggcca

SEQ ID NO: 139 - DNA, 20 bp, synthetic construct
  source 1..20: mol_type = other DNA
agcatcacggctatgacacc

SEQ ID NO: 140 - DNA, 20 bp, synthetic construct
  source 1..20: mol_type = other DNA
tcacttgcacatccaggcaa

SEQ ID NO: 141 - DNA, 26 bp, synthetic construct
  source 1..26: mol_type = other DNA
agtctccataatcattgttgagaaca

SEQ ID NO: 142 - DNA, 403 bp, Cannabis sativa
  source 1..403: mol_type = other DNA
  misc_difference 202: note = a or c
tgcttctcagagggagtgtgtgtgagagagaagtggccgaagcccgtggagtgtgttgttgaggtgggccgtaccgatggagacgagtgcccaaacgacgtcgttcggagaacaacgcggtgccggaggtaagctcaagaggccggctcgcaagccaccgtcaactccctactctcgtcctccggcgctaccgacccaaatngataggggtcagcggcgatggttgtccaagctcgttgatcccgcataccgcctcattagtgatggcgcctccatcttctttccgaatttcttctcaaaatcacctttcaccattgatgccaacactgaagatcacggtgggttttacatatatattcaactctctatgcttcttagtcaagctttctttcgattgattagg

SEQ ID NO: 143 - DNA, 403 bp, Cannabis sativa
  source 1..403: mol_type = other DNA
  misc_difference 202: note = t or c
agctatactgtaatatccctcattgatttgtataataaacttgaatcagagaattgatgaaggttgtaccaaattttagaaaatctctgtttgataattttcccatggttatatggtgtagtgtagaaaaagtgttattgaatactatgttgtttttggtgttttgtggttgaagtttctgtttgtgtttggttttccagtntctcacagagcacaagcatttactggaacgtatggaataaattacgggagaattgcagataacatcccttctccggatgaagttgctactcttctcagagcagctaagataaagaatgttagaatatacgatgcagatcacagtgttctcaaggcctttagtgggactggacttgaattagtaattggacttccaaatgga

SEQ ID NO: 144 - DNA, 403 bp, Cannabis sativa
  source 1..403: mol_type = other DNA
  misc_difference 202: note = a or g
tctcgtgaaaacacttccactcatctttcgtccacccattcttgcatgccgcttatttggtatttgtggcagagggtacattgacatatttgttgtgcaacatgatgactgccatccaccgctcccccatttgtaacactgtcgagaaacaccagtgcaggagcaaaccggcactggcatgatagactcatcaaaagttatnaggttcaagcccacatcctgactatcccattctgttctatactttgtgccatcagaggtagctcgtctgttcagatcctctgctactttctttcgcggtggctttgactccctggatgaatgtcccttgttcacctttggtcgctttgtctgaggtgacttgatagcatcagaagatataactgtaatggggaaggcatcg

SEQ ID NO: 145 - DNA, 403 bp, Cannabis sativa; identical to SEQ ID NO: 1
  source 1..403: mol_type = other DNA
  misc_difference 202: note = g or a

SEQ ID NO: 146 - DNA, 403 bp, Cannabis sativa
  source 1..403: mol_type = other DNA
  misc_difference 202: note = g or t
cgtgaagctgtttcccttgatgactcagtcccaatcgagcacacgacttgagaacaaacgggtacgtgaacttatcgggtcgggtcgggctgttacgagccatatagaggaaaaagtgtagagctttgattgggtcggggctccgagagtaggcacggatcatggtgttgtagtggaatgaattgggggatgggatagagtngagaatgagacgggcataagtgaggtcgccattggaggaaagagctgagaaggtgaagatgttggtaaggctcttttgttgggttttgcctgatttgagtgtttgggcatggagttgcagagcttcgggcatggagtttatctccatcttcggaggtggtggcggcgccgccaatgtgcatggcagagctccgatgagagt

SEQ ID NO: 147 - DNA, 403 bp, Cannabis sativa
  source 1..403: mol_type = other DNA
  misc_difference 202: note = a or c
tataattatgcttataataccatttttatcacctttctccaatctttagttctctgattaatttacttcctataaacagagtaacttcgagagagagggagagatatatatcttttttgtgccaccgcttcactcacataccattccatgtcgtgaagtgcatataggattaggtccatccacgtagctagcgaaatatccntgatatttttagcacaaaaacagatattgtccaaatttctctataaaaagcaacgaatcttatattcttcctctacttgcccaaaatagtaataatggatagagctttaactggtttgatctgctgcttcgtccttacggtggtcatctttacagagactgcagctgcagcaatacataccgtaggagaccacataggctg

SEQ ID NO: 148 - DNA, 403 bp, Cannabis sativa
  source 1..403: mol_type = other DNA
  misc_difference 202: note = c or g
tgaaaaagattgtggacaatattataactcaaaaacgtataatttaaaataattaatacatatagtatactactttttctcttttcacaacacgtgtatcattttgtggtgctctcttctcttgtgtctaattattatataaaggatagaacaagttccaaaacataataaaacccaccgagtgctaattaataaagttctnaagctctcttcttcttcttgcttctcctctacttggccaaaataatagtttgagagaaaaagaggtgggaagggacgacctttgaacacagttgattttcataatggcaagaggtttagtgggtttgatctgctgtttcgcccttacggtggtcatctttacagatagtacagcggcggtgacacatacggttggagacac

SEQ ID NO: 149 - DNA, 403 bp, Cannabis sativa
  source 1..403: mol_type = other DNA
  misc_difference 202: note = c or a
cgcttttagtatttatattgtaccaactcaattatcatgaacatttgtttcatctttacccgttatgtacttgtagggtattgcaaagatgggctgaccatgtccttcatatagaacacaacttgtggaggtatcactatttttcaattttgttttcacgctagattcttctttcaacgtgttctgagccgatcaaggtccnatgcagggctctgattttgtatgttgcggctggagcactgccagaggcattggcagctcttcgtgaagcacaacaaccagacacagctgccatgtttgtccttgcttgccgtgaaatacactcagaaatcatctccagtttggacttggatgatgaaactagttcctctaccaatgagaaactgcttaagttgcctggttt

SEQ ID NO: 150 - DNA, 403 bp, Cannabis sativa; identical to SEQ ID NO: 6
  source 1..403: mol_type = other DNA
  misc_difference 202: note = g or a

SEQ ID NO: 151 - DNA, 403 bp, Cannabis sativa; identical to SEQ ID NO: 11
  source 1..403: mol_type = other DNA
  misc_difference 202: note = g or c

SEQ ID NO: 152 - DNA, 403 bp, Cannabis sativa; identical to SEQ ID NO: 19
  source 1..403: mol_type = other DNA
  misc_difference 202: note = a or g

SEQ ID NO: 153 - DNA, 403 bp, Cannabis sativa
  source 1..403: mol_type = other DNA
  misc_difference 202: note = t or c
ttaacgaccagaaaagttcagggggcatgatttaatacatgtcaaagttcggagagtaaaaatcctaattagcctaaaaaaaactaagcaagacagtactgatatatatttatatgtttatataggtgctcatacatttggaaggggtcaatgtggaaccttcagtggaagattattcaattttagtggcacaggtgctccngatccaaccatagaggaaaactacttgagattattacaagatctatgtcctcaaggaggaaacgcctctatcttaacagatttagaccccaccacaccagatttgtttgacagaaactactactcaaatcttcaacaacacatgggattgttcacaagtgaccaagagctgttttcaagcccattagcagcaaatgacact

SEQ ID NO: 154 - DNA, 403 bp, Cannabis sativa
  source 1..403: mol_type = other DNA
  misc_difference 202: note = g or a
aacataaaagagattgcagaaaacttttccaaggtactcttataaacagtcataattaatcacagatttgttagaacaattatgctcacttcctaaaactcaacgatgaatttggctgtacattgtaatacagactgattgattttatctttctctatcgacgcagcttgcttgcaatgcacatacaatttgtgataatganttgagacccctggggaccgggctgtttcctgtcatatctattattaaccacaggtaactagatcagttcaataatgctgtttaacaaattttagttcattgatatgttttaggtaccattctcctgatttcagtctgttgaatgtttaagctgcttgccaaattctgttctggtttttgagggaaagttagctgtcgtgcg

SEQ ID NO: 155 - DNA, 403 bp, Cannabis sativa
  source 1..403: mol_type = other DNA
  misc_difference 202: note = c or t
tttggtgcgaaagttttgaatttacagttttgtcccttttctgttcttattttatttttacaatctgaaactgaagaagaagaagaagaagatagatactgagttcggaccgagtcaattatggattctaactcagatacaacgagtcctaaaacaccgcttttgggaccagaagctcggagaccgagtgataacggtcgcnggaatcgccgactcagtcgaagatactctgttaactcgctcaggaccgaattcgtttccaggctcccggaaaaagtccgttctggtttcgacgttgagtccagtccttatgatatcgacctaactcggaccactggcttgagcggaggtttgtttgaaatttcaattcaagaaaaaaaacaatactctgtttggttactga

SEQ ID NO: 156 - DNA, 403 bp, Cannabis sativa
  source 1..403: mol_type = other DNA
  misc_difference 202: note = g or a
agaggaagctagtgacgaagatgcacacttgacataaaccgaaatcaacgaattcaaaaccgaagtaacaaaacacaatccgaacttcacaaccgcgcaatgcatttgttgacactgcctttcatcatcaactacaagtccaaaagcactaagaacactagataaagtaaagtcatcaaccccaacactaaccctcctcatntcatgaaatgtttgaatagcagcaaagccatcattgttatgagaacaacaagtgatcatggcattgtagaaaacagtgtctctcatggtcaatggagtagcaaagaacacttcccttgccaacttaagattcccagctacagagtaagctgcaatcatagtcgttctagaaatgatatctggtttaggaatttggtcgaac

SEQ ID NO: 157 - DNA, 403 bp, Cannabis sativa
  source 1..403: mol_type = other DNA
  misc_difference 202: note = c or a
tgggaaatacggctcttcatttggctgctttgattgggaacattcctatgtgtcgggccatcaccaaaccctattctgatcaggcagcttcgttgattgaaattgagaataatatgggtgaaactcctctcttcttggcttctcttcatggcaagaaggacacttttctttatcttaactcaatcttgaagaaaactcggcntgaaaccgcgattacttgttgtagaaggcataatggtgatactgttctacactgtgctatagatggcgagttcttcggtaagtctaagtagaatttattacttcttttaaattaattctcattgaacatgctcttaattgtaaattgaattttgaattgaaattaaactcaaaaattaattgtcaattaaaattttgttac

SEQ ID NO: 158 - DNA, 403 bp, Cannabis sativa
  source 1..403: mol_type = other DNA
  misc_difference 202: note = g or a
aaacagttcgtaaatgtatgtatttataggcaaggtcgggtggctcggtggacctgaattgttaagaacacgtcggatcaagttattattgaatgtctcagcattttcaagattggcatccggttctttggcaccaccgagccatggccaaccagcttcggtaacaacaacaggaatccccgaaaaatttgcagcctccatngcataataggtagcatccaccattgcatcaaacatgctactatagtggaaaagagtgttgggatcaacaatctgcttaaccgagggaagtggtcgaaaaatagcataatcaatcggaaaaatgccatccccttccgtgtatccataatatggatacgcatttaacatgtaataggaatttgtgtttttcaaaaactgaagg

SEQ ID NO: 159 - DNA, 403 bp, Cannabis sativa
  source 1..403: mol_type = other DNA
  misc_difference 202: note = a or g
agaggactaaaatacttgcatcaagattgtagtgcggcgattttacattttgacataaaacctcataacattcttctagattttgatttttctcctaaaatttctgacttcggccttgccaagttgtggcaaagggatgggagtggtgtatcactgttgaagggtagaggcacaattggatatacagcaccggagatgcatnacagaaattttggtgaagtatcttataaatctaatgtttatagttatggaatgatggttttagaaatggtgggcggaagaaaaaatattgatactagtgtttctcgaacaagtgcaatatattatccacattgggcacataagcatatcaatgatgatgatgatggtgagctcttgaaaaatatttgtgaagaaataatgg

SEQ ID NO: 160 - DNA, 403 bp, Cannabis sativa
  source 1..403: mol_type = other DNA
  misc_difference 202: note = c or t
ataaatctaatgtttatagttatggaatgatggttttagaaatggtgggcggaagaaaaaatattgatactagtgtttctcgaacaagtgcaatatattatccacattgggcacataagcatatcaatgatgatgatgatggtgagctcttgaaaaatatttgtgaagaaataatggaaaatgaagatgatgaaatgttggngagaaagatgattattgtaagcttatggtgcattcaagctatgccatccaatcgtccctccatgtttaatgtcgtacaaatgatggaagggagtttagaatctttacatatgccaccaaaaccaacattgtctttacaaaaagagtaatattatttgtcatctatatatatatatatttgtattttcatacaacttctagt

SEQ ID NO: 161 - DNA, 403 bp, Cannabis sativa
  source 1..403: mol_type = other DNA
  misc_difference 202: note = g or a
ttgagaatactgaaagcgcaatcagaataccggtggggaatcgataagaacctgccggtttgttcttccacatccactcctgagcatgctgctgatgcccctgaaatcacacacattaagaattaatttgtggtatcgaattctaaacactaaaaaaaccgaaaaaggaatgaaagatttgggggctctttttacctgcatnttttgatgacccatcattcccgaagcctgatactggaatattacgtttaatggtcagtaatcggtaataatgacaattctattaaatctcttatgctacttaaaaaacaaaagagaaaaagaactgacagtcgaagataaagaaggccgtggcagctgctgttgaggctgcatttggaactgtgtttgatgctgtatctgc

SEQ ID NO: 162 - DNA, 403 bp, Cannabis sativa
  source 1..403: mol_type = other DNA
  misc_difference 202: note = g or c
atgactacgatagtattttatacctgcagctgcaatacacatggtttgaccatcatcatcatccccaatggcatataggaaatacccaccaagatgtttcttagcatactcgaccttttcatttatagattctgaagcttcgtagccataccaagtatttttatcagccttgtacatgcacttggtaacatccttatcatanatcccaccttctggatcaggaatatctttatacgcgatgggacccgaataggcaatagttggtgccccaatgctggaatcattttcatcagccaatgtccatttatttccgtacaagggaatgcccataactagtttacaggtcgaaaattcatttgtagtgagccaactattaatagagtcttcactgctgttattaggg

SEQ ID NO: 163 - DNA, 20 bp, synthetic construct
  source 1..20: mol_type = other DNA
ccgtggagtgtgttgttgag

SEQ ID NO: 164 - DNA, 20 bp, synthetic construct
  source 1..20: mol_type = other DNA
cccaccgtgatcttcagtgt

SEQ ID NO: 165 - DNA, 20 bp, synthetic construct
  source 1..20: mol_type = other DNA
cgtggagtgtgttgttgagg

SEQ ID NO: 166 - DNA, 23 bp, synthetic construct
  source 1..23: mol_type = other DNA
cccatggttatatggtgtagtgt

SEQ ID NO: 167 - DNA, 20 bp, synthetic construct
  source 1..20: mol_type = other DNA
gtccagtcccactaaaggcc

SEQ ID NO: 168 - DNA, 24 bp, synthetic construct
  source 1..24: mol_type = other DNA
tcccatggttatatggtgtagtgt

SEQ ID NO: 169 - DNA, 20 bp, synthetic construct
  source 1..20: mol_type = other DNA
tccagtcccactaaaggcct

SEQ ID NO: 170 - DNA, 20 bp, synthetic construct
  source 1..20: mol_type = other DNA
ttcgtccacccattcttgca

SEQ ID NO: 171 - DNA, 20 bp, synthetic construct
  source 1..20: mol_type = other DNA
catccagggagtcaaagcca

SEQ ID NO: 172 - DNA, 20 bp, synthetic construct
  source 1..20: mol_type = other DNA
atccagggagtcaaagccac

SEQ ID NO: 173 - DNA, 21 bp, synthetic construct
  source 1..21: mol_type = other DNA
cggtcatattttccggcgttc

SEQ ID NO: 174 - DNA, 20 bp, synthetic construct
  source 1..20: mol_type = other DNA
ggagagactcaaggcggttc

SEQ ID NO: 175 - DNA, 20 bp, synthetic construct
  source 1..20: mol_type = other DNA
tattttccggcgttcctccg

SEQ ID NO: 176 - DNA, 20 bp, synthetic construct
  source 1..20: mol_type = other DNA
tcagtcccaatcgagcacac

SEQ ID NO: 177 - DNA, 20 bp, synthetic construct
  source 1..20: mol_type = other DNA
ctgcaactccatgcccaaac

SEQ ID NO: 178 - DNA, 20 bp, synthetic construct
  source 1..20: mol_type = other DNA
tctgcaactccatgcccaaa

SEQ ID NO: 179 - DNA, 20 bp, synthetic construct
  source 1..20: mol_type = other DNA
tttttgtgccaccgcttcac

SEQ ID NO: 180 - DNA, 21 bp, synthetic construct
  source 1..21: mol_type = other DNA
cagcctatgtggtctcctacg

SEQ ID NO: 181 - DNA, 20 bp, synthetic construct
  source 1..20: mol_type = other DNA
gcctatgtggtctcctacgg

SEQ ID NO: 182 - DNA, 21 bp, synthetic construct
  source 1..21: mol_type = other DNA
tgtggtgctctcttctcttgt

SEQ ID NO: 183 - DNA, 20 bp, synthetic construct
  source 1..20: mol_type = other DNA
tccaaccgtatgtgtcaccg

SEQ ID NO: 184 - DNA, 22 bp, synthetic construct
  source 1..22: mol_type = other DNA
tggtgctctcttctcttgtgtc

SEQ ID NO: 185 - DNA, 20 bp, synthetic construct
  source 1..20: mol_type = other DNA
gggctgaccatgtccttcat

SEQ ID NO: 186 - DNA, 20 bp, synthetic construct
  source 1..20: mol_type = other DNA
atttcacggcaagcaaggac

SEQ ID NO: 187 - DNA, 23 bp, synthetic construct
  source 1..23: mol_type = other DNA
agcagtttctcattggtagagga

SEQ ID NO: 188 - DNA, 24 bp, synthetic construct
  source 1..24: mol_type = other DNA
cagataggacactctgaacaagct

SEQ ID NO: 189 - DNA, 20 bp, synthetic construct
  source 1..20: mol_type = other DNA
tcaggctcgaatgcaagaga

SEQ ID NO: 190 - DNA, 23 bp, synthetic construct
  source 1..23: mol_type = other DNA
cagataggacactctgaacaagc

SEQ ID NO: 191 - DNA, 20 bp, synthetic construct
  source 1..20: mol_type = other DNA
cagtggagtctctggtgaca

SEQ ID NO: 192 - DNA, 20 bp, synthetic construct
  source 1..20: mol_type = other DNA
ggctagcccgctttatcaca

SEQ ID NO: 193 - DNA, 21 bp, synthetic construct
  source 1..21: mol_type = other DNA
tcagtggagtctctggtgaca

SEQ ID NO: 194 - DNA, 20 bp, synthetic construct
  source 1..20: mol_type = other DNA
tagcccgctttatcacaggg

SEQ ID NO: 195 - DNA, 21 bp, synthetic construct
  source 1..21: mol_type = other DNA
acctcggagaccatgtcattg

SEQ ID NO: 196 - DNA, 20 bp, synthetic construct
  source 1..20: mol_type = other DNA
tggggaaacaggagaagcac

SEQ ID NO: 197 - DNA, 20 bp, synthetic construct
  source 1..20: mol_type = other DNA
ggtttggttggggaaacagg

SEQ ID NO: 198 - DNA, 20 bp, synthetic construct
  source 1..20: mol_type = other DNA
ccagaaaagttcagggggca

SEQ ID NO: 199 - DNA, 20 bp, synthetic construct
  source 1..20: mol_type = other DNA
caaatctggtgtggtggggt

SEQ ID NO: 200 - DNA, 20 bp, synthetic construct
  source 1..20: mol_type = other DNA
aaatctggtgtggtggggtc

SEQ ID NO: 201 - DNA, 20 bp, synthetic construct
  source 1..20: mol_type = other DNA
ctctatcgacgcagcttgct

SEQ ID NO: 202 - DNA, 20 bp, synthetic construct
  source 1..20: mol_type = other DNA
cgcacgacagctaactttcc

SEQ ID NO: 203 - DNA, 20 bp, synthetic construct
  source 1..20: mol_type = other DNA
tctctatcgacgcagcttgc

SEQ ID NO: 204 - DNA, 20 bp, synthetic construct
  source 1..20: mol_type = other DNA
ctgagttcggaccgagtcaa

SEQ ID NO: 205 - DNA, 20 bp, synthetic construct
  source 1..20: mol_type = other DNA
aaacaaacctccgctcaagc

SEQ ID NO: 206 - DNA, 20 bp, synthetic construct
  source 1..20: mol_type = other DNA
actgagttcggaccgagtca

SEQ ID NO: 207 - DNA, 20 bp, synthetic construct
  source 1..20: mol_type = other DNA
caagccagtggtccgagtta

SEQ ID NO: 208 - DNA, 20 bp, synthetic construct
  source 1..20: mol_type = other DNA
gaacttcacaaccgcgcaat

SEQ ID NO: 209 - DNA, 22 bp, synthetic construct
  source 1..22: mol_type = other DNA
agctgggaatcttaagttggca

SEQ ID NO: 210 - DNA, 21 bp, synthetic construct
  source 1..21: mol_type = other DNA
agttggcaagggaagtgttct

SEQ ID NO: 211 - DNA, 20 bp, synthetic construct
  source 1..20: mol_type = other DNA
ggctcttcatttggctgctt

SEQ ID NO: 212 - DNA, 20 bp, synthetic construct
  source 1..20: mol_type = other DNA
acttaccgaagaactcgcca

SEQ ID NO: 213 - DNA, 21 bp, synthetic construct
  source 1..21: mol_type = other DNA
gacttaccgaagaactcgcca

SEQ ID NO: 214 - DNA, 20 bp, synthetic construct
  source 1..20: mol_type = other DNA
gctcggtggacctgaattgt

SEQ ID NO: 215 - DNA, 20 bp, synthetic construct
  source 1..20: mol_type = other DNA
cacggaaggggatggcattt

SEQ ID NO: 216 - DNA, 20 bp, synthetic construct
  source 1..20: mol_type = other DNA
acggaaggggatggcatttt

SEQ ID NO: 217 - DNA, 20 bp, synthetic construct
  source 1..20: mol_type = other DNA
tcaagattgtagtgcggcga

SEQ ID NO: 218 - DNA, 20 bp, synthetic construct
  source 1..20: mol_type = other DNA
ttcttccgcccaccatttct

SEQ ID NO: 219 - DNA, 21 bp, synthetic construct
  source 1..21: mol_type = other DNA
catcaagattgtagtgcggcg

SEQ ID NO: 220 - DNA, 20 bp, synthetic construct
  source 1..20: mol_type = other DNA
agaaatggtgggcggaagaa

SEQ ID NO: 221 - DNA, 21 bp, synthetic construct
  source 1..21: mol_type = other DNA
acaatgttggttttggtggca

SEQ ID NO: 222 - DNA, 22 bp, synthetic construct
  source 1..22: mol_type = other DNA
agacaatgttggttttggtggc

SEQ ID NO: 223 - DNA, 20 bp, synthetic construct
  source 1..20: mol_type = other DNA
tgctgatgcccctgaaatca

SEQ ID NO: 224 - DNA, 20 bp, synthetic construct
  source 1..20: mol_type = other DNA
acacagttccaaatgcagcc

SEQ ID NO: 225 - DNA, 20 bp, synthetic construct
  source 1..20: mol_type = other DNA
caaatgcagcctcaacagca

SEQ ID NO: 226 - DNA, 21 bp, synthetic construct
  source 1..21: mol_type = other DNA
gcagctgcaatacacatggtt

SEQ ID NO: 227 - DNA, 20 bp, synthetic construct
  source 1..20: mol_type = other DNA
atgattccagcattggggca

SEQ ID NO: 228 - DNA, 20 bp, synthetic construct
  source 1..20: mol_type = other DNA
gcagctgcaatacacatggt

(19) United States
(12) Patent Application Publication
(10) Pub. No.: US 2025/0194482 A1
(43) Pub. Date: Jun. 19, 2025

(54) QUANTITATIVE TRAIT LOCI ASSOCIATED WITH PURPLE COLOR IN CANNABIS

(71) Applicant: PUREGENE AG, Zeiningen (CH)

(72) Inventors: Claudio CROPANO, Zeiningen (CH); Dániel Árpád CARRERA, Zeiningen (CH); Gavin Mager GEORGE, Zeiningen (CH); Leron KATSIR, Zeiningen (CH); Maximilian Moritz VOGT, Zeiningen (CH); Mercedes THIEME, Zeiningen (CH); Michael Eduard RUCKLE, Zeiningen (CH); Michele WYLER, Zeiningen (CH)

(21) Appl. No.: 18/848,873

(22) PCT Filed: Mar. 29, 2023
(86) PCT No.: PCT/IB2023/053121

§ 371 Date: Sep. 19, 2024

(30) Foreign Application Priority Data
Mar. 29, 2022 (GB) 2204468.9

(51) Int. Cl. A01H 1/04; A01H 1/00; C12Q 1/6827; C12Q 1/6895

(52) U.S. Cl. CPC A01H 1/045; A01H 1/107; C12Q 1/6827; C12Q 1/6895; C12Q 2600/13; C12Q 2600/156

(57) ABSTRACT

Provided herein are methods of characterizing and identifying a Cannabis spp. plant comprising quantitative trait loci (QTLs) associated with a purple color trait of interest, and methods of producing plants having a purple color trait of interest based on defined allelic states of polymorphisms defining the QTLs. Also provided are Cannabis spp. plants with the purple color trait of interest comprising defined allelic states of polymorphisms defining the QTLs and plants identified, characterized or produced by the methods described herein. Further provided are methods of marker assisted selection, genomic selection and marker assisted breeding, in particular using a combination of specific markers provided, for obtaining plants having a purple color trait of interest.

### BACKGROUND OF THE INVENTION

The present invention describes methods of identifying a Cannabis spp. plant comprising quantitative trait loci (QTLs) associated with purple color, and to Cannabis spp. plants comprising the QTLs. The invention also relates to plants with increased levels of purple color identified by the methods. The invention further relates to marker assisted selection and marker assisted breeding methods for obtaining plants having purple color, as well as to methods of producing Cannabis spp. plants with the absence of purple color and/or varying degrees of purple color and plants produced by these methods.

Modern Cannabis is derived from the cross hybridization of three biotypes; Cannabis sativa L. ssp. indica, Cannabis sativa L. ssp. sativa, and Cannabis sativa L. ssp. ruderalis. Cannabis was divergently bred into two distinct, albeit tentative types, called Hemp and HRT (high-resin-type) Cannabis, respectively, which are typically used for different purposes. Hemp is primarily used for industrial purposes, for example in feed, food, seed, fiber, and oil production. Conversely, HRT cannabis is largely cultivated and bred for high concentrations of the pharmacological constituents, cannabinoids, derived from resin in the trichomes. Biomass, including the leaf and stem, of cannabis can also be an important source of cannabinoids.

Cannabis is the only species in the plant kingdom to produce phytocannabinoids. Phytocannabinoids are a class of terpenoid acting as antagonists and agonists of mammalian endocannabinoid receptors. The pharmacological action is derived from this ability of phytocannabinoids to disrupt and mimic endocannabinoids. Due to its psychoactive properties, one cannabinoid, delta-9-tetrahydrocannabinol (THC), the decarboxylation product of the plant-produced delta-9-tetrahydrocannabinolic acid (THCA), has received much attention in illegal or unregulated breeding programs, with modern HRT varieties having THC concentrations of 0.5% to 30%.

Cannabis can display a multitude of colors in its leaves, stem and inflorescence. Purple color displayed by some cannabis strains is an important characteristic for visual appeal in markets for HRT Cannabis. Purple Haze, for example, is named and marketed, in part, for the purple color of its inflorescence. Purple color of flowers is also an undesirable trait in some cases, some consumers prefer HRT Cannabis flowers that are light or dark green that show no purple. This makes flower color an important trait for HRT cannabis breeders, producers, and consumers. Selection of cannabis with or without purple color can be challenging as breeders may have to wait for the emergence of the purple color, especially in flowers, toward the end of a plant's life cycle. The purple color in cannabis plants is most likely the product of anthocyanin accumulation.

Anthocyanins are water-soluble flavonoids. This class of small molecules absorb specific wavelengths of the electromagnetic spectrum depending on their chemical structure. The absorbance of blue-green wavelengths of light by anthocyanins in plants can result in the appearance of purple color. Anthocyanin accumulates in the vacuole of epidermal cells conferring a range of colors, dark blue, purple, and reds, to plants. These colors can serve to attract pollinators and animal herbivores for seed dispersal. Anthocyanins may play important roles in plant stress mitigation to cold and drought, for example, by dampening the effect of reactive oxygen species. This suggests that purple color in cannabis plants may be an important trait for stress tolerance in HRT and Hemp cannabis.

The biosynthesis of anthocyanins has been well characterized in several plant species, though not in Cannabis. Anthocyanins are formed, like other flavonoids, from the coupling of three molecules of malonyl-CoA with 4-coumaryl CoA by Chalcone synthase to form naringenin chalcone. The isomerization of naringen chalcone is then catalyzed by chalcone isomerase (CHI) to naringenin. Naringenin is then oxidized by successive enzymes flavanone hydroxylase, flavonoid 3′-hydroxylase, and flavonoid 3′,5′-hydroxylase. The products of these oxidations are then converted to colorless leucoanthocyanidins by dihydroflavonol 4-reductase (DFR) and subsequently to colored anthocyanidins by anthocyanidin synthase (ANS). Sugar molecules are then coupled to the unstable anthocyanidins by various members of the glycosyltransferase enzyme family, resulting in stable anthocyanins.

Anthocyanin biosynthesis can be induced by developmental cues in response to abiotic and biotic stress. MYB transcription factors, R2R3-MYBs and R3-MYBs, have been demonstrated to play roles in the regulation of anthocyanin biosynthesis, and in secondary metabolism in general, in many agronomically important plant species. MYB transcription factors can act as positive regulators of anthocyanin production, such as MYB10 that can regulate skin color of apple varieties by activating the expression of genes that encode proteins for anthocyanin biosynthesis. MYB transcription factors also act as negative regulators of anthocyanin biosynthesis. For example, the R2R2-Myb of Brassica rapa, BrMYB4 inhibits anthocyanin accumulation by repressing the expression of cinnamate 4-hydroxylase, required for the biosynthesis of 4-coumaryl CoA.

The genetic basis for the accumulation or absence of purple color in cannabis is not known. While anthocyanin accumulation is a likely cause of the presence of purple color, the mechanisms underlying its regulation are unclear. Though MYB transcription factors have been shown to play a role in the regulation of anthocyanin accumulation in other plants species, the size of this family of transcription factors and the diversity of the activities of its members make it impossible to infer the role of MYB transcription factors in Cannabis. In many plant species, fruit or flower color can be affected by unwanted excessive browning in tissue rich in anthocyanins, caused by polyphenol oxidase catalyzing the degradation of anthocyanins to brown break down products. Understanding the genetic basis of purple color in cannabis can benefit the cannabis industry through elimination or inclusion of this trait to meet consumer preference. Regulation of this trait may also be important for developing climate resistant HRT and Hemp type cannabis varieties. The identification of molecular markers for this trait can facilitate acceleration of breeding times for varieties selecting for multiple traits. The present invention relates to markers and the identity of putative genes for the control of purple color accumulation in cannabis.

### SUMMARY OF THE INVENTION

The present invention relates to methods of characterizing and identifying a Cannabis spp. plant comprising quantitative trait loci (QTLs) associated with a purple color trait of interest, and to methods of producing plants having a purple color trait of interest based on defined allelic states of polymorphisms defining the QTLs. Also provided are Cannabis spp. plants with a purple color trait of interest comprising defined allelic states of polymorphisms defining the QTLs and plants identified, characterized or produced by the methods described. The invention further relates to methods of marker assisted selection, genomic selection and marker assisted breeding, in particular using a combination of specific markers provided herein, for obtaining plants having a purple color trait of interest or for modulating the purple color trait of Cannabis spp. plants. Also provided are quantitative trait loci that control a purple color trait in Cannabis spp., wherein the quantitative trait loci are defined by single nucleotide polymorphisms defined herein or genetic markers linked to the QTLs, as well as genes and polymorphisms likely responsible for regulating a purple color trait in a Cannabis spp. plant.

According to a first aspect of the present invention there is provided a method for characterizing a Cannabis spp. plant with respect to a purple color trait, the method comprising the steps of: (i) genotyping at least one plant with respect to a purple color QTL by detecting one or more polymorphisms associated with the purple color trait as defined in any of Tables 1 to 4 and 7 to 8; and (ii) characterizing the one or more plants with respect to the purple color QTL, based on the genotype at the polymorphism.

In a first embodiment of the method for characterizing a Cannabis spp. plant with respect to a purple color trait, the polymorphism may be selected from the group consisting of “common_4519”, “common_4525”, “common_4500”, “common_4513”, “rare_551”, “common_4487”, “common_4504”, “common_4516”, “rare_556”, “GBScompat_common_878”, “GBScompat_rare_165”, and combinations thereof, as defined in any one of Tables 1 to 4 and 7 to 8. These markers have all been validated for their predictive value for the purple color QTL and trait.

In a second embodiment of the method for characterizing a Cannabis spp. plant with respect to a purple color trait, the genotyping may be performed by any PCR-based detection method using molecular markers, by sequencing of PCR products containing the one or more polymorphisms, by targeted resequencing, by whole genome sequencing, or by restriction-based methods, for detecting the one or more polymorphisms.

According to a third embodiment of the method for characterizing a Cannabis spp. plant with respect to a purple color trait, the molecular markers may be for detecting polymorphisms at regular intervals within the purple color QTL such that recombination can be excluded. In an alternative embodiment, the molecular markers may be for detecting polymorphisms at regular intervals within the purple color QTL such that recombination can be quantified to estimate linkage disequilibrium between a particular polymorphism and the purple color phenotype. It will be appreciated by those of skill in the art that several possible markers may be designed for detecting the polymorphisms. For example, molecular markers may be for detecting polymorphisms such that recombination events can be detected to a resolution of 10′000 or 100′000 or 500′000 base pairs within the QTL. In one embodiment, the molecular markers may be designed based on a context sequence for the polymorphism provided in Tables 1 to 4 or 10 or the molecular markers may be selected from the primer pairs as defined in Table 5 or 11.

In a fourth embodiment of the method for characterizing a Cannabis spp. plant with respect to a purple color trait, the purple color QTL may be a quantitative trait locus selected from the group consisting of: 1) a QTL having a sequence that corresponds to nucleotides 68717484 to 77040783 of NC_044377.1 with reference to the CS10 genome and which is defined by one or more polymorphisms associated with purple color as defined in any one of Tables 1 to 4 and 7 to 8; 2) a QTL defined by, or centered on, a single nucleotide polymorphism at position 80922439 of NC_044373.1 with reference to the CS10 genome; or 3) a QTL defined by, or centered on, a single nucleotide polymorphism at position 6600328 of NC_044374 with reference to the CS10 genome. In another embodiment, the purple color QTL may be defined by a genetic marker linked to any of the aforementioned QTLs.

According to a second aspect of the present invention, there is provided for a method of producing a Cannabis spp. plant having a purple color trait of interest, the method comprising the steps of: (i) providing a donor parent plant having in its genome a purple color QTL characterized by one or more polymorphisms associated with the purple color trait of interest as defined in any one of Tables 1 to 4 and 7 to 8; (ii) crossing the donor parent plant having the purple color QTL with at least one recipient parent plant to obtain a progeny population of Cannabis spp. plants; (iii) screening the progeny population of Cannabis spp. plants for the presence of the purple color QTL; and (iv) selecting one or more progeny plants having the purple color QTL, wherein the mature plant displays the purple color trait of interest. In this way, the trait can be selected for in a plant using the purple color QTLs and markers therefor described herein.

In a first embodiment of the method of producing a Cannabis spp. plant having a purple color trait of interest, the method may further comprise the steps of: (v) crossing the one or more progeny plants with the donor recipient plant; and/or (vi) selfing the one or more progeny plants.

According to a second embodiment of the method of producing a Cannabis spp. plant having a purple color trait of interest, the screening step may comprise genotyping at least one plant from the progeny population with respect to the purple color QTL by detecting one or more polymorphisms associated with the purple color trait of interest as defined in any one of Tables 1 to 4 and 7 to 8.

In a third embodiment of the method of producing a Cannabis spp. plant having a purple color trait of interest, the method may comprise a step of genotyping the donor parent plant with respect to the purple color QTL, by detecting one or more polymorphisms associated with the purple color trait of interest as defined in any one of Tables 1 to 4 and 7 to 8, preferably prior to step (i).

According to a fourth embodiment of the method of producing a Cannabis spp. plant having a purple color trait of interest, the genotyping may be performed by a PCR-based detection method using molecular markers, by sequencing of PCR products containing the one or more polymorphisms, by targeted resequencing, by whole genome sequencing, or by restriction-based methods, for detecting the one or more polymorphisms.

In some embodiments of the method of producing a Cannabis spp. Plant having a purple color trait of interest, the molecular markers may be for detecting polymorphisms at regular intervals within the purple color QTL such that recombination can be excluded or such that recombination can be quantified to estimate linkage disequilibrium between a particular polymorphism and a purple color trait of interest. For example, molecular markers may be for detecting polymorphisms such that recombination events can be detected to a resolution of 10′000 or 100′000 or 500′000 base pairs within the QTL. It will be appreciated by those of skill in the art that several possible markers may be designed for detecting the polymorphisms. In one embodiment, the molecular markers may be designed based on a context sequence for the polymorphism described in any one of Tables 1 to 4 and 10 or may be selected from the primer pairs defined in Table 5 or 11.

According to a further embodiment of the method of producing a Cannabis spp. plant having a purple color trait of interest, the purple color QTL is a purple color presence QTL, or a purple color absence QTL defined by the allelic state of the polymorphisms as provided in any of Tables 1 to 4 or 7 to 8. In one embodiment, the purple color trait of interest is a purple color presence trait, and the purple color QTL is a purple color presence QTL. Of particular use in producing a Cannabis spp. plant having a purple color trait of interest, are the polymorphisms selected from the group consisting of “common_4519”, “common_4525”, “common_4500”, “common_4513”, “rare_551”, “common_4487”, “common_4504”, “common_4516”, “rare_556”, “GBScompat_common_878”, “GBScompat_rare_165”, and combinations thereof, as defined in any one of Tables 1 to 4 and 7 to 8, which have been validated for their predictive value for the purple color QTL and trait.

In one embodiment of the method of producing a Cannabis spp. plant having a purple color trait of interest, the purple color QTL may be a quantitative trait locus selected from the group consisting of: 1) a QTL having a sequence that corresponds to nucleotides 68717484 to 77040783 of NC_044377.1 with reference to the CS10 genome and which is defined by one or more polymorphisms associated with purple color as defined in any one of Tables 1 to 4 and 7 to 8; 2) a QTL defined by, or centered on, a single nucleotide polymorphism at position 80922439 of NC_044373.1 with reference to the CS10 genome; or 3) a QTL defined by, or centered on, a single nucleotide polymorphism at position 6600328 of NC_044374 with reference to the CS10 genome. In another embodiment, the purple color QTL may be defined by a genetic marker linked to any of the aforementioned QTLs.

According to a third aspect of the present invention there is provided for a method of producing a Cannabis spp. plant that has a purple color trait of interest, the method comprising introducing a purple color QTL characterized by one or more polymorphisms associated with the purple color trait of interest as defined in any one of Tables 1 to 4 and 7 to 8 into a Cannabis spp. plant, wherein said purple color QTL is associated with the purple color trait of interest in the plant.

In one embodiment of the method of producing a Cannabis spp. plant comprising a purple color trait of interest, introducing the purple color QTL may comprise crossing a donor parent plant having the purple color QTL characterized by one or more polymorphisms associated with the purple color trait of interest with a recipient parent plant. In an alternative embodiment of the method of producing a Cannabis spp. plant comprising a purple color trait of interest, introducing the purple color QTL characterized by one or more polymorphisms associated with the purple color trait of interest may comprise genetically modifying the Cannabis spp. plant. Several methods of genetic modification are known to those of skill in the art, including targeted mutagenesis, genome editing, and gene transfer. For example, a purple color QTL comprising one or more of the polymorphisms associated with the purple color trait of interest as defined in any one of Tables 1 to 4 and 7 to 8 herein may be introduced into a plant by mutagenesis and/or gene editing. In particular, the methods of genetically modifying a plant may be selected from the group consisting of CRISPR-Cas9 targeted gene editing, heterologous gene expression using various expression cassettes; TILLING, and non-targeted chemical mutagenesis using e.g., EMS. Alternatively, a Cannabis spp. plant may be transformed with a cassette containing the purple color QTL associated with the purple color trait of interest or a part thereof, via any transformation method known in the art.

In one embodiment of the method of producing a Cannabis spp. plant that has a purple color trait of interest, the purple color QTL is a quantitative trait locus selected from the group consisting of: 1) a QTL having a sequence that corresponds to nucleotides 68717484 to 77040783 of NC_044377.1 with reference to the CS10 genome and which is defined by one or more polymorphisms associated with purple color as defined in any one of Tables 1 to 4 and 7 to 8; 2) a QTL defined by, or centered on, a single nucleotide polymorphism at position 80922439 of NC_044373.1 with reference to the CS10 genome; or 3) a QTL defined by, or centered on, a single nucleotide polymorphism at position 6600328 of NC_044374 with reference to the CS10 genome. In another embodiment, the purple color QTL may be defined by a genetic marker linked to any of the aforementioned purple color QTLs.

According to a fourth aspect of the present invention there is provided for a Cannabis spp. plant characterized according to any method of characterizing a Cannabis spp. plant with respect to a purple color trait as described herein or produced according to the method of producing a Cannabis spp. plant having a purple color trait of interest as described herein. In some embodiments, the Cannabis spp. plant characterized according to the method of characterizing a Cannabis spp. plant having a purple color trait of interest as described herein or produced according to the method of producing a Cannabis spp. plant having a purple color trait of interest as described herein is not exclusively obtained by means of an essentially biological process.

In yet a further aspect of the present invention there is provided for a Cannabis spp. plant comprising a purple color QTL as described herein or characterized by one or more polymorphisms associated with a purple color trait of interest as defined in any one of Tables 1 to 4 and 7 to 8. In some embodiments, the plant is not exclusively obtained by means of an essentially biological process.

According to another aspect of the present invention there is provided for a quantitative trait locus that controls a purple color trait in Cannabis spp., wherein the quantitative trait locus is defined by, or centered on, a single nucleotide polymorphism at position 80922439 of NC_044373.1 with reference to the CS10 genome or a genetic marker linked to the QTL; or wherein the quantitative trait locus is defined by, or centered on, a single nucleotide polymorphism at position 6600328 of NC_044374 with reference to the CS10 genome or a genetic marker linked to the QTL; or wherein the quantitative trait locus has a sequence that corresponds to nucleotides 68717484 to 77040783 of NC_044377.1 with reference to the CS10 genome; and wherein the QTL is defined by one or more polymorphisms associated with a purple color trait as defined in any one of Tables 1 to 4 and 7 to 8 or a genetic marker linked to the QTL. In some embodiments, the quantitative trait locus may be provided as an isolated nucleic acid molecule(s). The invention further includes a genomic region defined by markers linked to the QTLs defined herein.

In yet a further aspect of the present invention there is provided for an isolated gene that controls a purple color trait in a Cannabis spp. plant, wherein the gene is selected from the group consisting of the genes as defined in Table 6 with reference to the CS10 genome. In one embodiment, the isolated gene has the gene identity number LOC115695758 and encodes a putative MYB Transcription factor, as defined in Table 6. In another embodiment, the isolated gene has the gene identity number LOC115725215 and encodes a putative GT1 domain transcription factor, as defined in Table 6. In a further embodiment, the isolated gene has the gene identity number LOC115695887 and encodes a putative GT1 domain transcription factor, as defined in Table 6. In yet another embodiment, the isolated gene has the gene identity number LOC115695872 or LOC115695871 and encodes an anthocyanidin 3-O-glucosyltransferase 2, as defined in Table 6.

### BRIEF DESCRIPTION OF THE FIGURES

Non-limiting embodiments of the invention will now be described by way of example only and with reference to the following figures:

FIG. 1: GWA of Purple Color in Cannabis in a F2 Population, GID 21 002 035 0000.

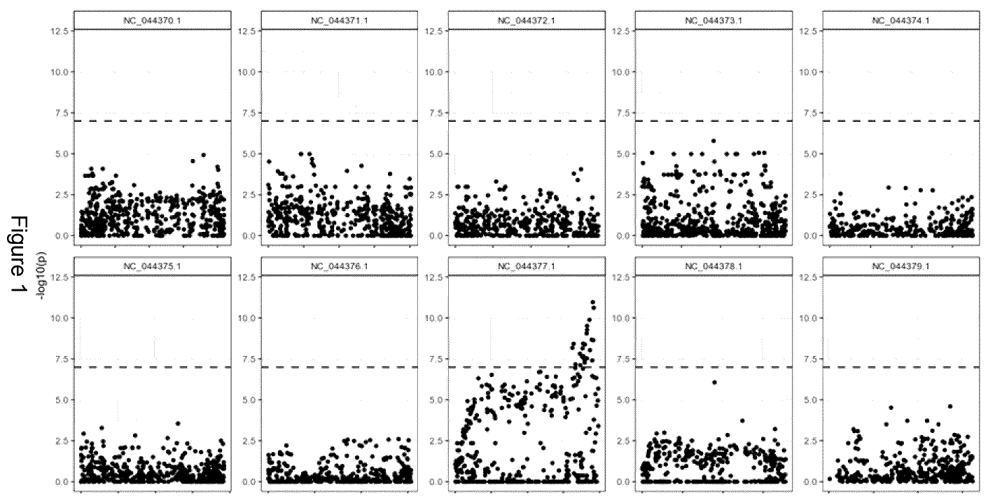

FIG. 2: GWA for Validation of Purple Color in Cannabis in a F2 Population, GID 21 002 035 0000.

FIG. 3: A multiple regression analysis with the allele as variable and purpleness as target using the random forest algorithm. The resulting R squares are derived from the comparison of the predictions from the developed model with the measured phenotype of the field grown training population. The points represent 100 permutations for “specific” (the 25 specific markers as described in Example 7) and 100 re-samplings of 25 random markers for “random”.

### SEQUENCES

The nucleic acid and amino acid sequences listed herein and in any accompanying sequence listing are shown using standard letter abbreviations for nucleotide bases, and the standard one- or three-letter abbreviations for amino acids. It will be understood by those of skill in the art that only one strand of each nucleic acid sequence is shown, but that the complementary strand is included by any reference to the displayed strand.

### DETAILED DESCRIPTION OF THE INVENTION

The present invention will now be described more fully hereinafter with reference to the accompanying drawings, in which some, but not all embodiments of the invention are shown.

The invention as described should not be limited to the specific embodiments disclosed and modifications and other embodiments are intended to be included within the scope of the invention. Although specific terms are employed herein, they are used in a generic and descriptive sense only and not for purposes of limitation.

As used throughout this specification and in the claims, which follow, the singular forms “a”, “an” and “the” include the plural form, unless the context clearly indicates otherwise.

The terminology and phraseology used herein is for the purpose of description and should not be regarded as limiting. The use of the terms “comprising”, “containing”, “having” and “including” and variations thereof used herein, are meant to encompass the items listed thereafter and equivalents thereof as well as additional items. It is, however, contemplated as a specific embodiment of the present disclosure that the term “comprising” encompasses the possibility of no further members being present, i.e., for the purpose of such an embodiment “comprising” is to be understood as having the meaning of “consisting of”.

Methods are provided herein for characterizing, identifying and obtaining plants having a purple color trait of interest prior to the plant displaying the color phenotypically, using a molecular marker detection technique. The inventors of the present invention have further produced and selected for purple colored Cannabis spp. plants by crossing plants displaying purple color with plants lacking purple color. Also demonstrated herein, the inventors were able to use genome wide association (GWA) to identify multiple QTLs linked to purple color. The inventors were also able to identify single nucleotide polymorphisms (SNPs) associated with the purple color trait; these SNPs were verified as genetic markers for identifying plants carrying the purple color trait of interest. The inventors used the methods described herein to identify candidate genes that are causative for the purple color trait. This finding provides for the improvement of methods for producing plants displaying differing degrees of purple color and plants that do not display purple color and modulating the purple color trait in Cannabis spp. plants. In addition, this finding provides a method of prescreening a population for the purple color trait.

A total of three QTLs for purple color were identified in the mixed populations tested and the two F2 populations tested.

Tables 1 to 4 and 7 to 8 herein provide several SNPs which define the QTLs associated with the purple color trait. In some embodiments one or more of the identified SNPs can be used to incorporate the purple color trait of interest from a donor plant, containing one or more of the QTLs associated with the trait, into a recipient plant. For example, the incorporation of the purple color trait of interest may be performed by crossing a donor parent plant to a recipient parent plant to produce plants containing a haploid genome from both parents. Recombination of these genomes provides F1 progeny where each haploid complement of chromosomes, of the diploid genome, is comprised of genetic material from both parents.

In some embodiments, methods of identifying one or more QTLs that are characterized by a haplotype comprising of a series of polymorphisms in linkage disequilibrium are provided. The QTLs each display limited frequency of recombination within the QTLs. Preferably the polymorphisms are selected from any one provided in Tables 1 to 4 and 7 to 8 herein, representing the purple color QTLs. Molecular markers may be designed for use in detecting the presence of the polymorphisms and thus the QTLs. Further, the identified QTLs and the associated molecular markers may be used in a cannabis breeding program to predict the purple color trait of plants in a breeding population and can be used to produce cannabis plants that display the purple color trait of interest, compared to the plants from which they are derived. The QTLs identified herein, and the markers associated with the QTLs, can be used to modulate the purple color trait in Cannabis spp. plants.

As used herein, reference to a “purple color” plant or a variety with a “purple color trait” refers to a plant or a variety that has the appearance of purple color at the time of harvest, as measured using the methods provided herein. In particular, a plant of purple color may accumulate a higher level of anthocyanin or anthocyanin-related compounds compared to a plant that does not have purple color at the time of harvest.

A “purple color trait of interest” refers to the state of the plant with respect to the purple color trait and includes the purple color absence trait and purple color presence trait.

A “purple color absence trait” is defined by the relative absence of purple color.

A “purple color presence trait” is defined by the relative presence of purple color.

The time of harvest is defined with respect to the maturity of the flower, where approximately greater than 50% of the pistils have turned brown in appearance. Alternatively, the time of harvest can also be determined by initiation of flowering for hemp-type cannabis or by other agronomic criteria common in the art.

It is a particular aim of the present invention to identify and characterize a plant for the purple color trait of interest early in the plant lifecycle, particularly prior to the plant displaying the purple color trait of interest. This can be achieved by genotyping the plant using molecular markers for detecting the QTL associated with the purple color trait prior to the time of harvest.

As used herein a “quantitative trait locus” or “QTL” is a polymorphic genetic locus with at least two alleles that differentially affect the expression of a continuously varying phenotypic trait when present in a plant or organism which is characterized by a series of polymorphisms in linkage disequilibrium with each other.

As used herein, the term “purple color QTL” or “purple color quantitative trait locus” refers to a quantitative trait locus characterized by one or more polymorphisms having an allelic state associated with the purple color trait of interest as described in any one of Tables 1 to 4 and 7 to 8, and in particular combinations of said polymorphisms.

In some cases, it is desirable to obtain a plant displaying a purple color presence trait. In other embodiments, it is desirable to obtain a plant displaying a purple color absence trait. Thus, it is an objective of the invention to provide for cannabis plants having a purple color presence QTL or a purple color absence QTL as described herein.

As used herein, “purple color presence QTL” or “purple color presence quantitative trait locus” refers to a quantitative trait locus characterized by one or more polymorphisms having an allelic state associated with the purple color presence trait, as described in Tables 1 to 4 and 7 to 8.

As used herein, “purple color absence QTL” or “purple color absence quantitative trait locus” refers to a quantitative trait locus characterized by one or more polymorphisms having an allelic state associated with the purple color absence trait, as described in Tables 1 to 4 and 7 to 8.

As used herein, “haplotypes” refer to patterns or clusters of alleles or single nucleotide polymorphisms that are in linkage disequilibrium and therefore inherited together from a single parent. The term “linkage disequilibrium” refers to a non-random segregation of genetic loci or markers. Markers or genetic loci that show linkage disequilibrium are considered linked.

As used herein, the term “purple color haplotype” refers to the subset of the polymorphisms contained within the purple color QTLs which exist on a single haploid genome complement of the diploid genome, and which are in linkage disequilibrium with the purple color trait.

As used herein, the term “donor parent plant” refers to a plant having a purple color haplotype, or one or more purple color alleles associated with the purple color trait of interest.

As used herein, the term “recipient parent plant” refers to a plant having a purple color haplotype, or one or more purple color alleles not associated with the purple color trait of interest.

The term “purple color allele” refers to the haplotype allele within a particular QTL that confers, or contributes to, the purple color trait of interest, or alternatively, is an allele that allows the identification of plants with the purple color trait or interest that can be included in a breeding program (“marker assisted breeding”, “marker assisted selection”, or “genomic selection”).

The term “crossed” or “cross” means the fusion of gametes via pollination to produce progeny (e.g., cells, seeds or plants). The term encompasses both sexual crosses (the pollination of one plant by another) and selfing (self-pollination, e.g., when the pollen and ovule are from the same, or genetically identical plant). The term “crossing” refers to the act of fusing gametes via pollination to produce progeny.

The term “GWAS” or “Genome wide association study” or “GWA” or “Genome wide association” as used herein refers to an observational study of a genome-wide set of genetic variants or polymorphisms in different individual plants to determine if any variant or polymorphism is associated with a trait, specifically the purple color trait.

As used herein a “polymorphism” is a particular type of variance that includes both natural and/or induced multiple or single nucleotide changes, short insertions, or deletions in a target nucleic acid sequence at a particular locus as compared to a related nucleic acid sequence. These variations include, but are not limited to, single nucleotide polymorphisms (SNPs), indel/s, genomic rearrangements, and gene duplications.

As used herein, the term “LOD score” or “logarithm (base 10) of odds” refers to a statistical estimate used in linkage analysis, wherein the score compares the likelihood of obtaining the test data if the two loci are indeed linked, to the likelihood of observing the same data purely by chance. The LOD score is a statistical estimate of whether two genetic loci are physically near enough to each other (or “linked”) on a particular chromosome that they are likely to be inherited together. A LOD score of 3 or higher is generally understood to mean that two genes are located close to each other on the chromosome. In terms of significance, a LOD score of 3 means the odds are 1,000:1 that the two genes are linked and therefore inherited together.

As used herein, the term “quantile-quantile” or “Q-Q” refers to a graphical method for comparing two probability distributions by plotting their quantiles against each other. If the two distributions being compared are similar, the points in the Q-Q plot will approximately lie on the line y=x. If the distributions are linearly related, the points in the Q-Q plot will approximately lie on a line, but not necessarily on the line y=x. Q-Q plots can also be used as a graphical means of estimating parameters in a location-scale family of distributions.

As used herein, a “causal gene” is the specific gene having a genetic variant (the “causal variant”) which is responsible for the association signal at a locus and has a direct biological effect on the purple color trait phenotype. In the context of association studies, the genetic variants which are responsible for the association signal at a locus are referred to as the “causal variants”. Causal variants may comprise one or more “causal polymorphisms” that have a biological effect on the phenotype.

The term “nucleic acid” encompasses both ribonucleotides (RNA) and deoxyribonucleotides (DNA), including cDNA, genomic DNA, isolated DNA and synthetic DNA. The nucleic acid may be double-stranded or single-stranded. Where the nucleic acid is single-stranded, the nucleic acid may be the sense strand or the antisense strand. A “nucleic acid molecule” or “polynucleotide” refers to any chain of two or more covalently bonded nucleotides, including naturally occurring or non-naturally occurring nucleotides, or nucleotide analogs or derivatives. By “RNA” is meant a sequence of two or more covalently bonded, naturally occurring or modified ribonucleotides. The term “DNA” refers to a sequence of two or more covalently bonded, naturally occurring or modified deoxyribonucleotides. By “cDNA” is meant a complementary or copy DNA produced from an RNA template by the action of RNA-dependent DNA polymerase (reverse transcriptase).

In some embodiments, the nucleic acid molecules of the invention may be operably linked to other sequences. By “operably linked” is meant that the nucleic acid molecules, such as those comprising the QTLs of the invention or gene(s) identified herein, and regulatory sequences are connected in such a way as to permit expression of the proteins when the appropriate molecules are bound to the regulatory sequences. Such operably linked sequences may be contained in vectors or expression constructs which can be transformed or transfected into plant cells or plants for expression. A “regulatory sequence” refers to a nucleotide sequence located either upstream, downstream or within a coding sequence. Generally regulatory sequences influence the transcription, RNA processing or stability, or translation of an associated coding sequence. Regulatory sequences include but are not limited to effector binding sites, enhancers, introns, polyadenylation recognition sequences, promoters, RNA processing sites, stem-loop structures, translation leader sequences and the like.

The term “promoter” refers to a DNA sequence that is capable of controlling the expression of a nucleic acid coding sequence or functional RNA. A promoter may be based entirely on a native gene, or it may be comprised of different elements from different promoters found in nature. Different promoters are capable of directing the expression of a gene at different stages of development, or in response to different environmental or physiological conditions. An “inducible promoter” is promoter that is active in response to a specific stimulus. Several such inducible promoters are known in the art, for example, chemical inducible promoters, developmental stage inducible promoters, tissue type specific inducible promoters, hormone inducible promoters, environment responsive inducible promoters.

The term “isolated”, as used herein means having been removed from its natural environment. Specifically, the nucleic acid or gene(s) identified herein may be isolated nucleic acids or gene(s), which have been removed from plant material where they naturally occur.

The term “purified”, relates to the isolation of a molecule or compound in a form that is substantially free of contamination or contaminants. Contaminants are normally associated with the molecule or compound in a natural environment, purified thus means having an increase in purity as a result of being separated from the other components of an original composition. The term “purified nucleic acid” describes a nucleic acid sequence that has been separated from other compounds including, but not limited to polypeptides, lipids and carbohydrates which it is ordinarily associated with in its natural state.

The term “complementary” refers to two nucleic acid molecules, e.g., DNA or RNA, which are capable of forming Watson-Crick base pairs to produce a region of double-strandedness between the two nucleic acid molecules. It will be appreciated by those of skill in the art that each nucleotide in a nucleic acid molecule need not form a matched Watson-Crick base pair with a nucleotide in an opposing complementary strand to form a duplex. One nucleic acid molecule is thus “complementary” to a second nucleic acid molecule if it hybridizes, under conditions of high stringency, with the second nucleic acid molecule. A nucleic acid molecule according to the invention includes both complementary molecules.

As used herein a “substantially identical” or “substantially homologous” sequence is a nucleotide sequence that differs from a reference sequence only by one or more conservative substitutions, or by one or more non-conservative substitutions, deletions, or insertions located at positions of the sequence that do not destroy or substantially alter the activity of the polypeptide encoded by the nucleic acid molecule. Alignment for purposes of determining percent sequence identity can be achieved in various ways that are within the knowledge of those with skill in the art. These include using, for instance, computer software such as ALIGN, Megalign (DNASTAR), CLUSTALW or BLAST software. Those skilled in the art can readily determine appropriate parameters for measuring alignment, including any algorithms needed to achieve maximal alignment over the full length of the sequences being compared. In one embodiment of the invention there is provided for a polynucleotide sequence that has at least about 80% sequence identity, at least about 90% sequence identity, or even greater sequence identity, such as about 95%, about 96%, about 97%, about 98% or about 99% sequence identity to the sequences described herein.

Alternatively, or additionally, two nucleic acid sequences may be “substantially identical” or “substantially homologous” if they hybridize under high stringency conditions. The “stringency” of a hybridisation reaction is readily determinable by one of ordinary skill in the art, and generally is an empirical calculation which depends upon probe length, washing temperature, and salt concentration. In general, longer probes required higher temperatures for proper annealing, while shorter probes require lower temperatures. Hybridisation generally depends on the ability of denatured DNA to re-anneal when complementary strands are present in an environment below their melting temperature. A typical example of such “stringent” hybridisation conditions would be hybridisation carried out for 18 hours at 65° C. with gentle shaking, a first wash for 12 min at 65° C. in Wash Buffer A (0.5% SDS; 2×SSC), and a second wash for 10 min at 65° C. in Wash Buffer B (0.1% SDS; 0.5% SSC).

Nucleotide positions of polymorphisms described herein are provided with reference to the corresponding position on the Cannabis sativa (assembly cs10) representative genome, provided as RefSeq assembly accession: GCF_900626175.2 on NCBI, loaded on 14 Feb. 2019, referred to herein as “cs10 reference genome” or “cs10 genome”.

### Methods of Identifying a QTL or Haplotype Responsible for the Purple Color Trait and Molecular Markers Therefor

In some embodiments, methods are provided for identifying a QTL or haplotype responsible for purple color trait and for selecting plants with the purple color trait of interest. In some embodiments, the methods may comprise the steps of:
a. Identifying a plant that displays the purple color trait phenotype within a breeding program.
b. Establishing a population by crossing the identified plant to itself (selfing) or a recipient parent plant.
c. Genotyping the resultant F1, or subsequent populations, for example, by sequencing methods.
d. Performing association studies, including phenotyping and linkage analysis, to discover QTLs and/or polymorphisms contained within the QTL.
e. Optionally, identifying cannabis paralogs of previously characterized genes that may be involved in the purple color phenotype.
f. Developing molecular markers that detect one or more polymorphisms linked to QTLs, alleles within these QTLs, or existing or induced polymorphisms.
g. Validating the molecular markers by determining the linkage disequilibrium between the marker and the purple color trait of interest.

### Trait Development and Introgression

In some embodiments, methods are provided for marker assisted breeding (MAB) or marker assisted selection (MAS) of plants having a purple color QTL or purple color trait. The methods may comprise the steps of:
a. Identifying a plant that displays the purple color trait of interest or phenotype or which contains a purple color QTL as defined herein.
b. Establishing a population by crossing the identified plant to itself (selfing) or another recipient parent plant.
c. Genotyping and phenotyping the resultant F1, or subsequent, populations, for example, by sequencing methods.
d. Performing association studies, inputting phenotype and genotype information to identify genomic regions enriched with polymorphisms associated with the purple color trait, to discover QTLs and/or polymorphisms contained within the QTL.
e. Optionally, identifying cannabis paralogs of previously characterized genes that may be involved in the purple color phenotype.
f. Developing molecular markers that detect one or more polymorphisms linked to QTLs, alleles within these QTLs, or existing or induced polymorphisms.
g. Using the molecular markers when introgressing the QTLs or polymorphisms into new or existing cannabis varieties to select plants containing the purple color haplotype or the purple color trait of interest.

### QTLs and Marker Assisted Breeding

In some embodiments, during the breeding process, selection of plants displaying the purple color trait may be based on molecular markers designed to detect polymorphisms linked to genomic regions that control the purple color trait of interest by either an identified or an unidentified mechanism. Previously identified genetic mechanisms may, for example, have a direct or pleiotropic effect on purple color in a plant. Examples include genes selected from: MYB transcription factors, such as R2R3-MYBs, R3-MYBs, MYB10, R2R2-MYBs, including BrMYB4. In some embodiments, QTLs containing such elements are identified using association studies. Knowledge of the mode-of-action is not required for the functional use of these genomic regions in a breeding program. Identification of regions controlling unidentified mechanisms may be useful in obtaining plants with the purple color trait of interest, based on identification of polymorphisms that are either linked to, or found within QTLs that are associated with the purple color trait of interest using association studies.

### Construction of Breeding Populations

Breeding populations are the offspring of sexual reproduction events between two or more parents. The parent plants (F0) are crossed to create an F1 population each containing a chromosomal complement of each parent. In a subsequent cross (F2), recombination has occurred and allows for mostly independent segregation of traits in the offspring and importantly the reconstitution of recessive phenotypes that existed in only one of the parental lines.

According to some embodiments, QTLs that lead to the phenotype of the purple color trait of interest are identified within synthetic populations of plants capable of revealing dominant, recessive, or complex traits. In one embodiment of the invention, a genetically diverse population of cannabis varieties, that are used to produce the synthetic population, are integrated into a breeding program by unnatural processes. In some embodiments, these processes result in changes in the genomes of the plants. The changes may include, but are not limited to, mutations and rearrangements in the genomic sequences, duplication of the entire genome (polyploidy), or activation of movement of transposable elements which may inactivate, activate or attenuate the activity of genes or genomic elements. According to one embodiment of the invention, the methods employed to integrate the plants into a breeding program include some or all of the following:
a. Growing plants in rich media or soils under artificial lighting;
b. Cloning of plants, often through a multitude of sub-cloning cycles;
C. Introduction of plants into in vitro, sterile growth environments, and subsequent removal to standard growth conditions;
d. Exposure to mutagens such as EMS, colchicine, silver nitrate, ethidium bromide, dinitroanalines, high concentrations mono or poly-chromatic light sources;
e. Growing plants under highly stressful conditions which include restricted space, drought, pathogen, atypical temperatures, and nutrient stresses.

### Purple Color Trait of Interest Association Studies and QTL Identification

In some embodiments, the synthetic populations created are either the offspring of the sexual reproduction or clones of plants in the breeding program such that genetic material of individuals in the synthetic populations is derived from one, or two, or more plants from the breeding program.

In one embodiment, plants identified within the synthetic population as having a purple color trait of interest may be used to create a structured population for the identification of the genetic locus responsible for the trait. The structured population may be created by crossing one (selfing) or more plants and recovering the seeds from those plants.

Plants in the structured population may be fully genotyped using genome sequencing to identify genetic markers for use in the association study (AS) database. Association mapping is a powerful technique used to detect quantitative trait loci (QTLs) specifically based on the statistical correlation between the phenotype and the genotype. In a population generated by crossing, the amount of linkage disequilibrium (LD) is reduced between genetic marker and the QTL as a function of genetic distance in cannabis varieties with similar genome structures. Simple association mapping is performed by biparental crosses of two closely related lines where one line has a phenotype of interest and the other does not. In some embodiments, advanced population structures may be used, including nested association mapping (NAM) populations or multi-parent advanced generation inter-cross (MAGIC) populations, however it will be appreciated that other population structures can also be effectively used. Biparental, NAM, or MAGIC structured populations can be generated and offspring, at F1 or later generations, may be maintained by clonal propagation for a desired length of time. In some embodiments, QTLs may be identified using the high-density genetic marker database created by genotyping the founder lines and structured population lines. This marker database may be coupled with an extensive phenotypic trait characterization dataset, including, for example, the purple color phenotype of the plants. Using the association studies described herein, together with accurate phenotyping, this method is able to identify genomic regions, QTLs and even specific genes or polymorphisms responsible for the purple color trait of interest that is directly introduced into recipient lines. Polygenic phenotypes may also be identified using the methods described herein.

In one embodiment, the structured population is grown to the time of harvest. To characterize the phenotypes of the lines, they are clonally reproduced so the phenotypic data can be collected in feasible replicates.

### Genomic Selection

In some embodiments, during the breeding process, selection of plants by genomic selection (GS) may be conducted. Genomic selection is a method in plant breeding where the genome wide genetic potential of an individual is determined to predict breeding values for those individuals. In some embodiments, the accuracy of genomic selection is affected by the data used in a GS model including size of the training population, relationships between individuals, marker density, use of pedigree information, and inclusion of known QTLs.

In some embodiments, a QTL or a SNP known to be associated with a trait that contributes to selection criteria can improve the accuracy of genomic selection models. In some embodiments, a genomic selection model that incorporates the purple color phenotype can be improved by the inclusion of the purple color QTL in the GS model. In some embodiments, the SNPs described in any of Tables 1 to 4 and 7 to 8 may be useful in a genomic selection model, and particularly in combination, for example where genotypes with unknown phenotypes are evaluated using an approach like a random forest algorithm for prediction of the purple color trait, and particularly in combination, to improve the predictive power of the model. In some embodiments, the SNPs described in any of Tables 1 to 4 and 7 to 8, and particularly combinations thereof, may be useful in a genomic selection model for the purple color trait to improve the predictive power of that model.

### Molecular Markers to Detect Polymorphisms

As used herein, the term “marker” or “genetic marker” refers to any sequence comprising a particular polymorphism or haplotype described herein that is capable of detection. For example, a marker may be a binding site for a primer or set of primers that is designed for use in a PCR-based method to amplify and thus detect a polymorphism or haplotype. Alternatively, the marker may introduce a restriction enzyme recognition site, or result in the removal of a restriction enzyme recognition site. Plants can be screened for a particular trait based on the detection of one or more markers confirming the presence of the polymorphism. Marker detection systems that may be used in accordance with the present invention include, but are not limited to polymerase chain reaction (PCR) followed by sequencing, Kompetitive allele specific PCR (KASP), restriction fragment length polymorphisms (RFLPs) analysis, amplified fragment length polymorphisms (AFLPs), cleaved amplified polymorphic sequences (CAPS), or any other markers known in the art.

In some embodiments “molecular markers” refers to any marker detection system and may be PCR primers, or targeted sequencing primers, such as those described in the examples below, more specifically the primers defined in Table 5 or 11. For example, PCR primers may be designed that consist of a reverse primer and two forward primers that are homologous to the part of the genome that contains a polymorphism but differ in the 3′ nucleotide such that the one primer will preferentially bind to sequences containing the polymorphism and the other will bind to sequences lacking it. The three primers are used in single PCR reactions where each reaction contains DNA from a cannabis plant as a template. Fluorophores linked to the forward primers provide, after thermocycling, a different relative fluorescent signal for homozygous and heterozygous alleles containing the polymorphism and for those lacking the polymorphism, respectively.

In some embodiments, allele-specific primers may each harbor a unique tail sequence that corresponds with a universal FRET (fluorescence resonant energy transfer) cassette. For example, the primer specific to the SNP may be labelled with a FAM and the other specific primer with a HEX dye. During the PCR thermal cycling performed with these primers, the allele-specific primer binds to the genomic DNA template and elongates, so attaching the tail sequence to the newly synthesized strand. The complement of the allele-specific tail sequence is then generated during subsequent rounds of PCR, enabling the FRET cassette to bind to the DNA. Alleles are discriminated through the competitive binding of the two allele-specific forward primers. At the end of the PCR reaction a fluorescent plate is read using standard tools which may include RT-PCR devices with the capacity to detect florescent signals and is evaluated with commercial software.

If the genotype at a given polymorphism site is homozygous, one of the two possible fluorescent signals will be generated. If the genotype is heterozygous, a mixed fluorescent signal will be generated. By way of example, genomic DNA extracted from cannabis leaf tissue at seedling stage can be used as a template for PCR amplifications with reaction mixtures containing the three primers. Final fluorescent signals can be detected by a thermocycler and analyzed using standard software for this purpose, which discriminates between individuals that are heterozygotes or homozygotes for either allele.

In some embodiments, molecular markers to one, two or more of the SNPs in the haplotype can be used to identify the presence of the QTL and by association, the purple color trait of interest.

Further, the QTL may include a number of individual polymorphisms in linkage disequilibrium, which constitute a haplotype and which, with high frequency, can be inherited from a donor parent plant as a unit. Therefore, in some embodiments, molecular markers can be utilized which have been designed to identify numerous polymorphisms which are in linkage disequilibrium with other polymorphisms, any of which can be used to effectively predict the phenotype of the offspring for the purple color trait of interest.

According to some embodiments, any polymorphism in linkage disequilibrium with one or more of the purple color QTLs can be used to determine the purple color haplotype in a breeding population of plants, as long as the polymorphism is unique to the purple color trait of interest in the donor parent plant when compared to the recipient parent plant.

In some embodiments the desired trait is the purple color presence trait, and the donor parent plant is a plant that has been genetically modified or selected to include a purple color presence QTL defined by a polymorphism conferring the purple color presence trait, for example any, some, or all of the polymorphisms defined in any one of Tables 1 to 4 and 7 to 8. Alternatively, the desired trait is the purple color absence trait, and the donor parent plant may be a plant that has been genetically modified or selected to include a purple color absence QTL defined by a polymorphism associated with the purple color absence trait, for example any, some, or all of the polymorphisms defined in any one of Tables 1 to 4 and 7 to 8.

In some embodiments, donor parent plants, as described above, are used as one of two parents to create breeding populations (F1) through sexual reproduction. In this embodiment, donor parent plants may be identified by detecting polymorphisms using the molecular markers as described above.

Methods for reproduction that are known in the art may be used. The donor parent plant provides the purple color trait of interest to the breeding population. The trait is made to segregate through the population (F2) through at least one additional crossing event of the offspring of the initial cross. This additional crossing event can be either a selfing of one of the offspring or a cross between two individuals, provided that each plant used in the F1 cross contains at least one copy of a desired QTL allele or haplotype.

In some embodiments, the purple color allele or purple color haplotype in plants to be used in the F1 cross is determined using the described molecular markers. In some embodiments, the resulting F2 progeny, or subsequent progeny, is/are screened for any of the polymorphisms associated with the purple color trait of interest described herein.

The plants at any generation can be produced by asexual means like cutting and cloning, or any method that yields a genetically identical offspring.

Production of Cannabis Spp. Plants Having the Purple Color Presence Trait

In some embodiments, a Cannabis spp. plant that does not have the purple color trait may be converted into a purple color plant according to the methods of the present invention by providing a breeding population where the donor parent plant contains a purple color presence QTL associated with the purple color presence trait, and recipient parent plant either displays the purple color absence trait or contains the purple color absence QTL.

In some embodiments the purple color presence trait may be introduced into a recipient parent plant by crossing it with a donor parent plant having the purple color presence QTL. In some embodiments the donor parent plant has a purple color phenotype and a contiguous genomic sequence characterized by one or more of the polymorphisms of any one of Tables 1 to 4 and 7 to 8 associated with the purple color allele or purple color haplotype conferring the purple color presence trait.

In some embodiments, the donor parent plant is any Cannabis spp. variety that is cross fertile with the recipient parent plant.

In some embodiments, MAS or MAB may be used in a method of backcrossing plants carrying the purple color presence trait to a recipient parent plant. For example, an F1 plant from a breeding population can be crossed again to the recipient parent plant. In some embodiments, this method is repeated.

In some embodiments, the resulting plant population is then screened for the purple color presence trait using MAS with molecular markers to identify progeny plants that contain one or more polymorphisms, such as those described in any one of Tables 1 to 4 and 7 to 8, indicating the presence of an allele of a QTL associated with the purple color presence phenotype. In another embodiment, the population of cannabis plants may be screened by any analytical methods known in the art to identify plants with desired characteristics, specifically purple color presence.

Production of Cannabis Spp. Plants Having the Purple Color Absence Trait

In some embodiments, a Cannabis spp. plant that has the purple color presence trait may be converted into a plant having a purple color absence trait according to the methods of the present invention by providing a breeding population where the donor parent plant contains a purple color absence QTL and the recipient parent plant either displays the purple color presence trait or contains a purple color presence QTL.

In some embodiments the purple color presence trait may be removed from a recipient parent plant by crossing it with a donor parent plant having the purple color absence QTL. In some embodiments, the donor parent plant does not have a purple color phenotype and contains a contiguous genomic sequence characterized by one or more of the polymorphisms of Tables 1 to 4 and 7 to 8 associated with the purple color allele or purple color haplotype conferring the purple color absence trait.

In some embodiments, the donor parent plant is any Cannabis spp. variety that is cross fertile with the recipient parent plant.

In some embodiments, MAS or MAB may be used in a method of backcrossing plants carrying the purple color absence trait to a recipient parent plant. For example, an F1 plant from a breeding population can be crossed again to the recipient parent plant. In some embodiments, this method is repeated.

In some embodiments, the resulting plant population is then screened for the purple color absence trait using MAS with molecular markers to identify progeny plants that contain one or more polymorphism, such as any of those described in Tables 1 to 4 and 7 to 8, indicating the presence of an allele of a QTL associated with the purple color absence phenotype. In another embodiment, the population of cannabis plants may be screened by any analytical methods known in the art to identify plants with desired characteristics, specifically purple color absence.

### Methods to Genetically Engineer Plants to Achieve the Purple Color Trait of Interest Using Mutagenesis or Gene Editing Techniques

Identifying QTLs, and individual polymorphisms, that correlate with a trait when measured in an F1, F2, or similar, breeding population indicates the presence of one or more causative polymorphisms in close proximity the polymorphism detected by the molecular marker. In some embodiments, the polymorphisms associated with the presence or absence of the purple color trait are introduced into a plant by other means so that a trait can be introduced into plants that would not otherwise contain associated causative polymorphisms or removed from plants that would otherwise contain associated causative polymorphisms. For example, the polymorphisms detailed in Tables 1 to 4 and 7 to 8 are molecular markers that can be used to indicate the presence of a possible causative polymorphism.

The entire QTLs or parts thereof which confer the purple color trait of interest described herein, or the genes or nucleic acid molecules described herein, may be introduced into the genome of a cannabis plant to obtain plants with a purple color trait of interest, through a process of genetic modification known in the art, for example, but not limited to, heterologous gene expression using an expression cassette including a sequence encoding the QTL(s) or part thereof, the gene(s), or the nucleic acids. The expression cassettes may contain all or part of the QTL(s) or gene(s), including possible causative polymorphisms.

The trait described herein may be introduced into, or removed from, the genome of a cannabis plant to obtain plants that include or exclude the causative polymorphisms and the potential to display a desired purple color trait of interest through processes of genetic modification known in the art, for example, but not limited to, CRISPR-Cas9 targeted gene editing, TILLING, non-targeted chemical mutagenesis using e.g., EMS.

The present invention further provides methods for producing a modified Cannabis spp. plant using genome editing or modification techniques. For example, genome editing can be achieved using sequence-specific nucleases (SSNs) the use of which results in chromosomal changes, such as nucleotide deletions, insertions or substitutions at specific genetic loci, particularly those associated with the purple color trait of interest described in Tables 1 to 4 and 7 to 8. Non limiting examples of SSNs include zinc finger nucleases (ZFNs), TAL effector nucleases (TALENs), meganucleases, and clustered regularly interspaced short palindromic repeats (CRISPR)/CRISPR-associated protein (Cas) system. In some embodiments, non-limiting examples of Cas proteins suitable for use in the methods of the present invention include Csnl, Cpfl Cas9, Cas 12, Cas 13, Cas 14, CasX and combinations thereof. In one embodiment, a modified Cannabis spp. plant having a purple color trait of interest is generated using CRISPR/Cas9 technology, which is based on the Cas9 DNA nuclease guided to a specific DNA target by a single guide RNA (sgRNA). For example, the genome modification may be introduced using guide RNA, e.g., single guide RNA (sgRNA) designed and targeted to introduce a polymorphism associated with the distinct sesquiterpene trait of interest, such as one or more polymorphism defined in Tables 1 to 4 and 7 to 8 or linked thereto.

DNA introduction into the plant cells can be performed using Agrobacterium infiltration, virus-based plasmid delivery of the genome editing molecules and mechanical insertion of DNA (PEG mediated DNA transformation, biolistics, etc.). In some embodiments, the Cas9 protein may be directly inserted together with a gRNA (ribonucleoprotein-RNP's) in order to bypass the need for in vivo transcription and translation of the Cas9+gRNA plasmid in planta to achieve gene editing. In one embodiment, a genome edited plant may be developed and used as a rootstock, so that the Cas protein and gRNA can be transported via the vasculature system to the top of the plant and create the genome editing event in the scion.

According to one embodiment of the present invention, the method of genetically modifying a plant may be achieved by combining the Cas nuclease (e.g., Cas9, Cpf 1) with a predefined guide RNA molecule (gRNA). The gRNA is complementary to a specific DNA sequence targeted for editing in the plant genome and which guides the Cas nuclease to a specific nucleotide sequence. The predefined gene-specific gRNAs may be cloned into the same plasmid as the Cas gene and this plasmid is inserted into plant cells as described above.

In some embodiments, once the gRNA molecule and Cas9 nuclease reach the specific predetermined DNA sequence, the Cas9 nuclease cleaves both DNA strands to create double stranded breaks leaving blunt ends. This cleavage site is then repaired by the cellular non homologous end joining DNA repair mechanism resulting in insertions or deletions which introduce a mutation at the cleavage site.

In one embodiment, a deletion form of the mutation may consist of at least 1 base pair deletion. As a result of this base pair deletion, the gene coding sequence for the putative gene(s) responsible for the purple color trait of interest, such as the genes described in Table 6, is disrupted and the translation of the encoded protein is compromised either by a premature stop codon or disruption of a functional or structural property of the protein.

In another embodiment, the purple color trait of interest in Cannabis spp. plants may be introduced by generating gRNA with homology to a specific site of predetermined genes in the Cannabis genome or a QTL defined herein. In one embodiment the gene may be one or more of the genes described in Table 6 herein. This gRNA may be sub-cloned into a plasmid containing the Cas9 gene, and the plasmid inserted into the Cannabis plant cells. In this way site specific mutations in the QTL are generated, including the SNPs associated with the purple color trait of interest described in Tables 1 to 4 and 7 to 8, and in particular a causative polymorphism, thus effectively introducing the purple color trait of interest into the genome edited plant.

In some embodiments, a modified Cannabis spp. plant exhibiting a purple color presence trait may be obtained using the targeted genome modification methods described above, wherein the plant comprises a targeted genome modification to introduce one or more polymorphisms associated with the purple color presence trait defined in Tables 1 to 4 and 7 to 8, wherein the modification effects the purple color presence trait.

In some embodiments, for example where the purple color trait of interest is a purple color absence trait, the genetic modification may be introduced using gene silencing, a process by which the expression of a specific gene product is lessened or attenuated. Gene silencing can take place by a variety of pathways, including by RNA interference (RNAi), an RNA dependent gene silencing process. In one embodiment, RNAi may be achieved by the introduction of small RNA molecules, including small interfering RNA (siRNA), microRNA (miRNA) or short hairpin RNA (shRNA), which act in concert with host proteins (e.g., the RNA induced silencing complex, RISC) to degrade messenger RNA (mRNA) in a sequence-dependent fashion. In particular, RNAi may be used to silence one or more of the putative causative genes described in Table 6 herein. Such RNAi molecules may be designed based on the sequence of these genes. These molecules can vary in length (generally 18-30 base pairs) and may contain varying degrees of complementarity to their target mRNA in the antisense strand. Some, but not all, RNAi molecules have unpaired overhanging bases on the 5′ or 3′ end of the sense strand and/or the antisense strand. As used herein, the term “RNAi molecule” includes duplexes of two separate strands, as well as single strands that can form hairpin structures comprising a duplex region. The RNAi molecules may be encoded by DNA contained in an expression cassette and incorporated into a vector. The vector may be introduced into a plant cell using Agrobacterium infiltration, virus-based plasmid delivery of the vector containing the expression cassette and/or mechanical insertion of the vector (PEG mediated DNA transformation, biolistics, etc.).

Plants may be screened with molecular markers as described herein to identify transgenic individuals with the purple color trait of interest or having a purple color QTL or polymorphism(s), following the genetic modification.

In some embodiments, Cannabis spp. plants having one or more of the polymorphisms of any one of Tables 1 to 4 and 7 to 8 associated with the purple color QTLs or linked thereto are provided. The polymorphisms, including possible causative polymorphisms, may be introduced, for example, by genetic engineering. In some embodiments the one or more polymorphisms associated with the purple color trait of interest or linked thereto are introduced into the plants by breeding, such as by MAS or MAB, for example as described herein.

The purple color QTLs described herein, or genes identified herein responsible for effecting the purple color trait, may be under the control of, or operably linked to, a promoter, for example an inducible promoter. Such QTLs or genes may be operably linked to the inducible promoter so as to induce or suppress the purple color trait or phenotype in the plant or plant cell.

Accordingly, in a further embodiment, Cannabis spp. plants comprising a purple color QTL described herein, including a purple color absence QTL or a purple color presence QTL, or one or more polymorphisms associated therewith, are provided. In some cases, such plants are provided for with the proviso that the plant is not exclusively obtained by means of an essentially biological process.

The following examples are offered by way of illustration and not by way of limitation.

### Example 1

### Genome-Wide Association Studies (GWAS) of Purple Flower Color in Cannabis

During outdoor field trials in 2020 it was observed that several populations of cannabis plants were comprised of individuals with varying degrees of purple-colored flowers. To identify molecular markers for the appearance of purple color in Cannabis the study was initially focused on the apical inflorescence in a diverse population comprising 3220 individuals.

Trimmed and dried apical inflorescence of Cannabis sativa genotypes were photographed and visually assessed for the presence of purple areas.

Individual plants whose apical inflorescence showed at least some purple areas were coded as 1, those only showing green areas were coded as 0.

DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit—96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified are provided in Table 5. The library pool was loaded onto Ion 550 chips with Ion Chef and sequenced with Ion GeneStudio S5 Plus according to the manufacturer's instructions (lon 550™ Kit from Thermo Fisher Scientific).

From a population of 3220 individuals, a genome-wide association study (GWAS) was performed to detect significant associations between genotypic information derived from targeted resequencing of the custom SNP marker panel described above and the appearance of purple color in the apical inflorescence. Flowers were coded as 1 for those showing at least some purple and 0 for those only showing green areas.

The genotypic matrix was filtered for SNPs having more than 30% missing values within the population and a minor allele frequency lower than 5%. This resulted in 2699 SNP markers after filtering. The GWAS was performed using GAPIT version 3 (Wang and Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The Blink model performed the best by the inventors' evaluation and was used for the analysis. SNPs surpassing an LOD (−log₁₀(p-value)) value of 5 were considered to have a significant association with trait variation.

SNPs showing a significant association with purple color in flower, with an LOD value greater than 5, were found on chromosome NC_044371.1, NC_044373.1, and NC_044377.1 with reference to the Cannabis Sativa CS10 genome and are listed in Table 1. The homozygous allele of the SNPs in Table 1 that can distinguish the presence of purple flower are listed along with their position and reference sequence. Interestingly the heterozygous state is also indicative of purple flower color, however less so than the homozygous state of the allele for purple flower color, indicating this is a dominant trait.

TABLE 1
SNPs associated with the purple color trait in flower field trial. The presence of the purple
color trait is predicted by the occurrence of the indicative allele (marked with *). The positions of
the SNPs are provided with reference to the CS10 reference genome as described herein.
“Homo_1” denotes the average phenotypic value associated with homozygous allele 1 based on
scoring for purple color from 0 to 1, where 1 indicated a purple plant and 0 indicated a green plant,
“Homo_2” denotes the average phenotypic value associated with homozygous allele 2 based on
scoring for purple color from 0 to 1, where 1 indicated a purple plant and 0 indicated a green plant
and ″Hetero″ denotes the average phenotypic value associated with heterozygous based on
scoring for purple color from 0 to 1, where 1 indicated a purple plant and 0 indicated a green plant.
BP refers to the nucleotide position of the SNP.

| | | | | Allele_ | Allele_ | Homo_ | Homo_ | | |
|---|---|---|---|---|---|---|---|---|---|
| SNP | Chromosome | BP | LOD | 1 | 2 | 1 | 2 | Hetero | Context Sequence |
| common_4485 | NC_044377.1 | 72948533 | 20.0423 | A | G* | 0.1 | 0.21 | 0.17 | CGGCTACGCTTTCGCCGGGGATAGCTCTCTTCCCGCGCCGGTCATATT |
| | | | | | | | | | TTCCGGCGTTCCTCCGGCGACAACCGCTACTGCCACCGCTTGGTCACC |
| | | | | | | | | | TTCCTTATCGTCTGCTCTCTACAAAGTCGATGGGTGGGGCGCACCTTAC |
| | | | | | | | | | TTCGCCGTCAACTCTTCCGGCAACGTCGCCGTTCGCCCTCACGGCGCT |
| | | | | | | | | | GGAACTTT[G/A]GCGCACCAGGAGATTGATTTGCTGAAAATTGTAAAGAA |
| | | | | | | | | | GGTTTCGGATCCGAAATCTAAGGGCGGGTTAGGTTTGCCCCTTCCGCT |
| | | | | | | | | | CGTTATTCGGCTTCCTGATGTGCTTAAGAACCGCCTTGAGTCTCTCCAG |
| | | | | | | | | | GCGGCGTTCGATTTCGCAATCCAGTCGCAGGACTATGAAAACCATTACC |
| | | | | | | | | | AGGGTGTTTACCCTGTG (SEQ ID NO: 1) |
| common_2262 | NC_044373.1 | 80922439 | 8.277951 | A* | G | 0.2 | 0.12 | 0.18 | TCAAGAAATAAGAATTAACTAAATATTGCCACTTAACCTAGTAAAATTAA |
| | | | | | | | | | GAGCAGTTTCACGTGTTAAAATTAAATAAAATAATGAACTCAACAGAAAC |
| | | | | | | | | | TTAAACCACAATACCTCCAACTTGTTGGGGGGTATTGGTTTTACATTCTC |
| | | | | | | | | | TACTTTCAATGTACAACAACCAACAGTATCTGCTTCATCATCATCCTGCA |
| | | | | | | | | | A[G/A]GCATGAGATCATAATGTCAGTGATGCCTTCAGTTAACAGCTAGGG |
| | | | | | | | | | GAACTTAAAGATGATGTAGAGAAACATGTTGAGCAACTTAAATGATGCA |
| | | | | | | | | | ATGTAGCAGATTCGAGCAACAAATATAAATCAAACACCTTTCATATTTCT |
| | | | | | | | | | TTTTCCTTAACAAGGGCACAAATAAACAATGACAAAAATGTTAAACTGAA |
| | | | | | | | | | GAGCTGT (SEQ ID NO: 2) |
| common_2032 | NC_044373.1 | 44537208 | 7.285258 | A* | C | 0.19 | 0.14 | 0.18 | CATGCAAATACATACATATACATATGTGATTTTTTTTTATGTATACTATAT |
| | | | | | | | | | ATATAGGTCATGGGGTGATGTGAATGAGTGCTTGGGAAGCAAGAGATC |
| | | | | | | | | | AAGTATCATAAATAATGGAGGGGAGTTAGGCCATGAATATCGTGATCAT |
| | | | | | | | | | CATCATCATCAGCATCGGCATATTGGGGTTTCAACGATGAAGATGAAGA |
| | | | | | | | | | AGAT[A/C]AAAGGGAGAAGGAAAGTGAGAGAACCAAGGTTTTGCTTTAA |
| | | | | | | | | | GACCATGAGCGAGGTCGATGTGCTTGATGATGGTTACAAGTGGAGAAA |
| | | | | | | | | | GTACGGACAGAAAGTGGTCAAGAACACACAGCATCCCAGGTACCTAATT |
| | | | | | | | | | AATATCCATTTATTCATTAATTTATAATATTAACAACTCTAACAATGCCATT |
| | | | | | | | | | AATATTAAAGC (SEQ ID NO: 3) |
| common_5220 | NC_044379.1 | 34679389 | 6.59189 | A* | G | 0.2 | 0.13 | 0.14 | GATTGCACTGTGAAAGGGATTCGAAGTAACGGCCGGATCCTCATGACG |
| | | | | | | | | | GTCTTGATCAATATGAGCAATTAATGTTCTAGGGTTTAGGCCTCTTGATT |
| | | | | | | | | | GATGGTGGTGATGGGCAGCCGCCATTGAAACGGAATTACTAGGGTTTT |
| | | | | | | | | | CTAATTCCCTGGTCGTAGTAGTGTTTGATGTAGTGGTTGCCATGAGAAA |
| | | | | | | | | | ATGGGA[A/G]GTAGGCTTTCGTTCTTGGATTATTTGGGTGGTGCTAGTAG |
| | | | | | | | | | TAGTACTACTACTCATTATAAGCTCTGGCTTATTATTCATGTCACAAAAT |
| | | | | | | | | | GAGTAGTGTGGTTTTGATGAGAAGAAGAAGCTGGGTGATGTTGGGTTAA |
| | | | | | | | | | TGAGTTCCCAGTTCCAAGGAGCAGGTAATAAACCTAGAAAGTCGTCCTC |
| | | | | | | | | | GGACTTTGACCCA (SEQ ID NO: 4) |
| common_816 | NC_044371.1 | 45301500 | 6.588524 | A | G* | 0.15 | 0.28 | 0.21 | TCCAATCTTTGGGAGCAACACTGTAAGATGTAACAGTGACACC |
| | | | | | | | | | TTCACTATTAGTGACTTCAAAGGATAGGGGTTGATTTTTTAAAT |
| | | | | | | | | | CAGCATTTATGTGCCAGTTTTGGCCCCAGTTCCTTCCCATTGA |
| | | | | | | | | | AAGCCAACCAGTTCTTGAACCCTTAATTTTCACAGCAGATACG |
| | | | | | | | | | TCTCCAGCACCGGCAACATTGCTGATTA[A/G]CACCGAAATGA |
| | | | | | | | | | AGATACTTGAACCGTCAATTGTGAATCGTATTCCTCCTTCCTTT |
| | | | | | | | | | CTGCACTTTATTCTGTTACAGAAGTCAAATTAACTAGTTGTCAA |
| | | | | | | | | | CATGCCACATTTTTTCCTAATAGATTTAATCACACCGAACATAG |
| | | | | | | | | | TTTTCGTCATCAATATGCCACATTTACAACAACATTGTCATCTC |
| | | | | | | | | | ATCTTTTTCACTAT (SEQ ID NO: 5) |
| common_4499 | NC_044377.1 | 74270487 | 6.51065 | A | G* | 0.04 | 0.21 | 0.13 | CGAAAAGCCTATCTGAAATCTTAGTTCTACAGATAGGACACTC |
| | | | | | | | | | TGAACAAGCTAATGAACAAGATTTACATACTGCAAAACAAAAT |
| | | | | | | | | | GATAAATATGATTAGTTTTAATTTAATGTTAAAAGTGAAATCAA |
| | | | | | | | | | GCATGTAGGATTCATAGGAATGATGATGACTGCTTACAACAAA |
| | | | | | | | | | AATGGCGACACGGCAAAAGAATTGCAGC[G/A]GTCGGGGATT |
| | | | | | | | | | CAAAACATACTTTACACATGTGAGAATTGGCATCTCCATTCCC |
| | | | | | | | | | CATTTGTTTCAGCTCCTTTTCCTTCATCTCTTGCATTCGAGCCT |
| | | | | | | | | | GAAAACAACATACATCTTGAGTTTGAGATACCTTAACACTTTTT |
| | | | | | | | | | CGAGAACTTTTTAGTTTCTTGATCAACAAATGCATTTTCCTTGT |
| | | | | | | | | | GGATTTAAAATTATGC (SEQ ID NO: 6) |
| common_4448 | NC_044377.1 | 68957824 | 6.199807 | A | G* | 0.16 | 0.18 | 0.15 | TTGATCAGCGAAGAAAGGCCAACCAATAATTGGCACTCCAGC |
| | | | | | | | | | GCTCAAACTCTCCAGTGTCGAGTTCCAACCACAATGCGATAAA |
| | | | | | | | | | AACCCTCCTATTGCAGGGTGGTTCAAAACCTCTTCTTGTGGAC |
| | | | | | | | | | ACCAACTACAAATCACACATCTTTCTCTTGTCTCTTCCACAAAC |
| | | | | | | | | | TCAGTTGGAATAATTCCCGAGTTTCCATC[G/A]ATGATGTCGG |
| | | | | | | | | | GTCTTATAATCCAAACAAAGGGTTTCCCACTGTTAGCCAAACC |
| | | | | | | | | | CCAAGCGAACTCAACAAGTTCGTCAGGTGTCATAGCCGTGAT |
| | | | | | | | | | GCTGCCGAAATTAACATAAATCACCGAATTGGCCTCCCTAGAA |
| | | | | | | | | | TTCAACCATTGCAAGCATTCAAGCTCTTCTTTCCATAGATTAGA |
| | | | | | | | | | TCCAATGGATGACAAACTT (SEQ ID NO: 7) |
| common_4452 | NC_044377.1 | 69326994 | 5.601726 | A* | G | 0.26 | 0.11 | 0.2 | GAGTGCCGTATATTTGTATTTAAACATTAGTCAACCAA |
| | | | | | | | | | TATGATCAAATGTATATATACGGTTACATATTACGCAT |
| | | | | | | | | | ATATATGAATCAAAGTTATATTACTTTCTCAATATGATC |
| | | | | | | | | | AAAGTTGTGATTTTGTTGGTGCTAGCCACACTCATTAA |
| | | | | | | | | | TCAAGTATGGAGTAGTACTACAAGTAATAATTATTGCA |
| | | | | | | | | | TAGAGAAGGA[G/A]AGACAAGCTCTTCTCAACTTGAAG |
| | | | | | | | | | AAAGGCTTTGTCGATGATGGCAATCGTCTATCCTCAT |
| | | | | | | | | | GGACAAGTAGTAGCCGTGATTGTTGTGCATGGAGAG |
| | | | | | | | | | GTATCAGGTGCGATAACTCAAAAACTCATCGTCATATT |
| | | | | | | | | | ATCGCTCTTGATCTTAAATCTGATGACAACAATCATAA |
| | | | | | | | | | TTATTTGGGTGGTGAAATTGGTCCTTCT (SEQ ID |
| | | | | | | | | | NO: 8) |
| GBScompat_ | NC_044370.1 | 519152 | 5.110202 | A* | G | 0.25 | 0.16 | 0.24 | GTCGCAAATGGAAATTTACGCCCGCGATGTACTCGAA |
| rare_2 | | | | | | | | | TTAAACCCATTAACCCCATTTCTCAGGTACTCCACAAA |
| | | | | | | | | | ATCATCATATTACTTTTTCTTTCTAATTTCACATTTTTTT |
| | | | | | | | | | GAATTTGTTTTTGGGTTTTGGTGGAAATAGGTGAAAG |
| | | | | | | | | | GATTGCCATTTAATCGGTATTCATGGCTAACAACCCA |
| | | | | | | | | | CAATGCGTTTGC[G/A]AAGCTGGGACAGAAATCGCAG |
| | | | | | | | | | ACGGGAACACCGATTGTGTCTTCCATGAATCAACAGG |
| | | | | | | | | | ACTCCATTACTAGCCAGCTCAATGTAAGTTTTTTTTTTT |
| | | | | | | | | | TTCTTTTTAGTTAGTGAAATTTATGTTGTTTGTTTCTCG |
| | | | | | | | | | GGAAAGTTTTGCGGTAAAATTAAGGGGAAAATATGAT |
| | | | | | | | | | CAATGACTGGACTTTACAATAACTAAAA (SEQ ID NO: 9) |

### Example 2

Genome-wide association studies (GWAS) of whole plant purple color in Cannabis It was observed that the purple color in cannabis is not restricted to the flowers alone. It can be found in leaves, stem, and other components of the shoot system of cannabis. The inventors thus sought to identify additional SNP markers associated with whole plant purpleness and to understand if the markers found to be associated with purple color in flowers were also relevant to the presence of purple color in the whole plant. They assessed purple color visually of the whole plant from a mixed population, that is a subset of the population used in Example 1, consisting of 2274 individuals.

At the time of harvest, plants were photographed, and genotypes were visually assessed for the presence of purple in the whole plant, the areas on leaf, stem, and flower. Plants showing at least some purple areas were coded as 1, those only showing green areas were coded as 0.

DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified are provided in Table 5. The library pool was loaded onto Ion 550 chips with Ion Chef and sequenced with Ion GeneStudio S5 Plus according to the manufacturer's instructions (lon 550™ Kit from Thermo Fisher Scientific).

From a population of 2274 individuals, a genome-wide association study (GWAS) was performed to detect significant associations between genotypic information derived from targeted resequencing of the custom SNP marker panel described above and the appearance of purple color in the whole plant. Plants were coded as 1 for those showing at least some purple and 0 for those only showing green areas.

The genotypic matrix was filtered for SNPs having more than 30% missing values within the population and a minor allele frequency lower than 5%. This resulted in 2350 SNP markers after filtering. The GWAS was performed using GAPIT version 3 (Wang and Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The Blink model performed the best by the inventors' evaluation and was used for the analysis. SNPs surpassing an LOD (−log₁₀(p-value)) value of 5 were considered to have a significant association with trait variation.

The inventors identified SNPs significantly associate with purple color in the whole plant on chromosome NC_044372.1, NC_044377.1, and NC_044378.1, listed in Table 2. They identified two SNP markers that were found in both experiments “common_4485” and “common_4448”, as well as 10 additional SNP markers. The new insight indicated that the same QTL on chromosome NC_044377.1 was associated with purple color in both the flower and the whole plant.

TABLE 2
SNPs associated with the purple color trait in a whole plant field trial. The presence of
the purple color trait is predicted by the occurrence of the indicative allele (marked with *). The
positions of the SNPs are provided with reference to the CS10 reference genome as described
herein. “Homo_1” denotes the average phenotypic value associated with homozygous allele 1
based on scoring for purple color from 0 to 1, where 1 indicated a purple plant and 0 indicated a
green plant, “Homo_2” denotes the average phenotypic value associated with homozygous allele
2 based on scoring for purple color from 0 to 1, where 1 indicated a purple plant and 0 indicated
a green plant and “Hetero” denotes the average phenotypic value associated with heterozygous
based on scoring for purple color from 0 to 1, where 1 indicated a purple plant and 0 indicated a
green plant. BP refers to the nucleotide position of the SNP.

| | | | | Allele_ | Allele_ | Homo_ | Homo_ | | |
|---|---|---|---|---|---|---|---|---|---|
| SNP | Chromosome | BP | LOD | 1 | 2 | 1 | 2 | Hetero | Context Sequence |
| common_4451 | NC_044377.1 | 69163028 | 17.16585 | A | C' | 0.07 | 0.25 | 0.15 | AAATGGCACCGCATCCGAAACAACAAATATCCCAATCAAACGA |
| | | | | | | | | | AAGAACTCACTTATTGCCTTATGACACCGTCTTGCTTCATCCCC |
| | | | | | | | | | ACTCTGATCATCGCTACCCGATGCTCCGAAATAACGCTTTCCA |
| | | | | | | | | | GCCACCATTCTCACCACCATGTTAAGAGTTAAATCTTCTAACCA |
| | | | | | | | | | ACGACTCAACTCAACTACAACTTTACA[A/C]GACTTGTAGAGCT |
| | | | | | | | | | CTCTAATCCCTACTTCAACCTCTGAAATCCTCACTTGCTTCAAC |
| | | | | | | | | | ATCTCTAGACGGCGGTTAGAGAGGAGTTCTAACGTGGCGATCT |
| | | | | | | | | | TCCTCATTTCGCGCCAAAAAGGGCTATAAGGTGCGAAACCAAA |
| | | | | | | | | | GACTGCGTAGTTGTAGCCCATGTGCTTGGCTGCCACGGTTGTA |
| | | | | | | | | | GGGCGCGAGGCCAGC (SEQ ID NO: 10) |
| common_4502 | NC_044377.1 | 74475495 | 11.38578 | C* | G | 0.21 | 0.15 | 0.19 | GATAATTTGATCTTACCTTGTTGTGTACCATAAGTAATCAGTGG |
| | | | | | | | | | AGTCTCTGGTGACATTTAACTGCTCTAAGAGACCAACTGCTGT |
| | | | | | | | | | GATTTTTGTATCTTCATCCACTGAAGACATGTCTTCACTAAATGT |
| | | | | | | | | | CTCCCATGAGTACAATGCACCTTTCAAGGGCGACATTTGCGTT |
| | | | | | | | | | CGAGTCATTTGTACTCTCACCTGTTA[G/C]GAAGTGACAGATGC |
| | | | | | | | | | CCGTTATAGACAATGAGTGAAGTAGCCAATGCATCTTACATATA |
| | | | | | | | | | GTACACGATCAACCCTGTGATAAAGCGGGCTAGCCCTGATATG |
| | | | | | | | | | GTATTATGTTCTTATATCAAGCTCAATAATTAATCGTTTTCTTAT |
| | | | | | | | | | GGCCCATTTAGTCTCAGGGTCAGTCCTGTTATAGTATTATGTTC |
| | | | | | | | | | TTACACATTAT (SEQ ID NO: 11) |
| GBScompat_ | NC_044377.1 | 73090006 | 8.571359 | A | G* | 0.17 | 0.25 | 0.35 | CTTCAGCCCAAACCTTTTGAGCAACCTCAAAAGTCCTATCAATA |
| rare_165 | | | | | | | | | CCATGCTCACCATCAAGTAAGATCTCTGGCTCAACAATTGGGA |
| | | | | | | | | | CCAAACCATTGTCCTAAACATAACAAAACATAATCAATTTTCACA |
| | | | | | | | | | AACACAAGTACAAAAATCAAGATTAGCTTAGTCTTGTCTGATTA |
| | | | | | | | | | GTTATCTTACTTGGGAGATAGCAGC[A/G]TAGCGGGCTAGACC |
| | | | | | | | | | CCAAGCGGCTTCCTTCACAGCCAGAGCTGATGGGCCATTGGG |
| | | | | | | | | | AATGCTCACAACAGTACGCCTGTTTAAATAATGTAATTTTGTGT |
| | | | | | | | | | CAAATACCGATTAAAGAGCATTGCTATAAGGCATTATAGTGTCC |
| | | | | | | | | | AACATCACTATTAGGCACTACGGGTGCTTTTTAACATTTCTCAA |
| | | | | | | | | | CCAGACAAAAGTA (SEQ ID NO: 12) |
| common_4500 | NC_044377.1 | 74383124 | 7 .631759 | A | C* | 0.11 | 0.25 | 0.19 | TCGACTTCAATGAGAAATCTCAACCCCTGTGGTAAGAATTTTG |
| | | | | | | | | | TGTACTTTTTTAAAAATTGAATTTTTAAAATTTCCCAGTTCGCTG |
| | | | | | | | | | AATATTTGTATTAACTGTGTCTAATTTTTCATACTAGACATTGAA |
| | | | | | | | | | AGAATGGTGTCTTTGAAGGGAATGGTTATCCGTTGCAGTTCAA |
| | | | | | | | | | TAATTCCGGAAATCAGAGAAGCAAT[C/A]TTTAGATGTCTTGTG |
| | | | | | | | | | TGCGGTTACTACTCTGAACCTGTTGCTGTAGAGAAAGGTAATT |
| | | | | | | | | | AGTTAGGATACCATTCGCATGGGCTAGTTTTTTCTTATCTATAT |
| | | | | | | | | | CACAATGTCATTCTAAATTATTTTCCCTTTTCAGGACGGATAAC |
| | | | | | | | | | TGAACCAACAAGATGCTTGAAGGAAGAATGCCAAGCAAGAAA |
| | | | | | | | | | CTCCATGACACTT (SEQ ID NO: 13) |
| common_4054 | NC_044377.1 | 7972754 | 7.433539 | A* | G | 0.17 | 0.15 | 0.23 | TTAACTTAATGATCATATAGATAGTAATAAACTATTAATTAATTA |
| | | | | | | | | | ATTTTGCTGGGGATGAGAATGGTGGCCAGGTAGCTTTTCCTT |
| | | | | | | | | | GATCTTTTCCATAACAGTTTTCTTCTCGTGAAGAGGAGTAGGA |
| | | | | | | | | | TCATGAGTAGTAGCAGTTGGTTCGTCCTTGTGTTTTCCAGTAA |
| | | | | | | | | | GTTTCTCTTTTATTTTTGTTCCTAAACC[C/T]TTCTTCTTCCTTCT |
| | | | | | | | | | CCCTCCAGCTCCATCATCCTCTGACTGCATCCACACCCATTTT |
| | | | | | | | | | ATAATTATTATATATATTAATCATTCATTTTTAATATATATAATTA |
| | | | | | | | | | CATTATATTATTATTTTAATATTTTAAAATGTAAATAAAAAATTTA |
| | | | | | | | | | GTGATTCAAATTATAATTTTTTATATTATATGTTGATAGCAATTT |
| | | | | | | | | | TGTTATT (SEQ ID NO: 14) |
| common_4448 | NC_044377.1 | 68957824 | 6.644629 | A | G* | 0.15 | 0.2 | 0.18 | TTGATCAGCGAAGAAAGGCCAACCAATAATTGGCACTCCAGC |
| | | | | | | | | | GCTCAAACTCTCCAGTGTCGAGTTCCAACCACAATGCGATAAA |
| | | | | | | | | | AACCCTCCTATTGCAGGGTGGTTCAAAACCTCTTCTTGTGGAC |
| | | | | | | | | | ACCAACTACAAATCACACATCTTTCTCTTGTCTCTTCCACAAAC |
| | | | | | | | | | TCAGTTGGAATAATTCCCGAGTTTCCATC[G/A]ATGATGTCGG |
| | | | | | | | | | GTCTTATAATCCAAACAAAGGGTTTCCCACTGTTAGCCAAACC |
| | | | | | | | | | CCAAGCGAACTCAACAAGTTCGTCAGGTGTCATAGCCGTGAT |
| | | | | | | | | | GCTGCCGAAATTAACATAAATCACCGAATTGGCCTCCCTAGAA |
| | | | | | | | | | TTCAACCATTGCAAGCATTCAAGCTCTTCTTTCCATAGATTAGA |
| | | | | | | | | | TCCAATGGATGACAAACTT (SEQ ID NO: 7) |
| common_4519 | NC_044377.1 | 76201790 | 5.94937 | A* | G | 0.26 | 0.09 | 0.19 | CGATCACTTCGTAGATGCATCCTCCCACAAGGTAGCACAATTGT |
| | | | | | | | | | AGAAAGTGCTAAATCATGCTTTATTCCATTTGTTCTTTTTGTCTTC |
| | | | | | | | | | TCTTTTTGCTTAATCGAACGATGTTGTGAACTTGTAGGGTTGTCA |
| | | | | | | | | | AATTCGAAGGGAGAGCGCACATGGAGAGAGCGTTTGTTGCAACA |
| | | | | | | | | | ATGTGAGGGCCCTGTTTGATGA[A/G]CTCCCAACTCCACACCTAA |
| | | | | | | | | | TTGTGGAGATCACACCATTCCCTGAAGGGCCTCTCACTGAAAAA |
| | | | | | | | | | GATTACACCAAAGCTGAGAAATTGGAGAGGGTACTTAGAACTGG |
| | | | | | | | | | CCCGAACGTTTGATTCTTCTCTCGAGTTAAATCATCGCTGTCTCT |
| | | | | | | | | | CGTTAGAACTACAGCTTAATTGTATGTATGTTTTGAGCCTTGTAC |
| | | | | | | | | | ATAT (SEQ ID NO: 15) |
| common_4599 | NC_044378.1 | 3495196 | 5.705698 | A | T* | 0.17 | 0.21 | 0.14 | CCATATAATGCAAATTCTCTAATATCAATTACAAAATCAAATATAA |
| | | | | | | | | | GACACAGATGCCTAAATGATGCCATGTAATTTCGATCACAGCATT |
| | | | | | | | | | GAATTTTTCTTCAAGATTGAAGAGTTAAGAAGTAAAGAACTTTAC |
| | | | | | | | | | CATAGATGTGTAGGAGACATTGTAACGAAAAAGAGGCGCTTCTT |
| | | | | | | | | | GCGAGGATCAACTTTAGTAGC[A/T]ACCCAATCTGCCCAAGCTTC |
| | | | | | | | | | CATAGCCAACTCCATGGCACCAACACCATCTAACTCTTCACAAAT |
| | | | | | | | | | TCCACTTTCATCAGAACTCCATCTGCATAATAAATTGGTTGCATA |
| | | | | | | | | | GTAAGTACAAAGTGCTCAACACATCATATTCTGAAACTTAACTCA |
| | | | | | | | | | AAATGTTACTATGATTCATTTTACTTACAACAGCTTAACTGGACCT |
| | | | | | | | | | (SEQ ID NO: 16) |
| common_1535 | NC_044372.1 | 63488008 | 5.52871 | A | G* | 0.144 | 0.19 | 0.23 | GAAGTATGGGAAAGAAAGAGCTTGAGTCAAGAAGCTTTGATTGA |
| | | | | | | | | | GGCATTCAAAATGGAAGTGAATCAAGTAAAAATGGAGTTAAGAA |
| | | | | | | | | | GTGCAAATGAGTTGGTGAGCCAGTTGATGGATGATGTTGAAATA |
| | | | | | | | | | CTTACTTATGATATACTAAAGGCGAAAACTGAGATTAATGAAATG |
| | | | | | | | | | AAGAGGAAAGAAACAGAAGCTCAA[A/G]TTGAGATAGCACTAATG |
| | | | | | | | | | AAAACTGAGCTTCAGAAAGAGAGAAGAGATTGCAATGATGGTCA |
| | | | | | | | | | CATAACGGCTTCACTCAAGGAATACGAGTCCTTGGTCAAAAAGG |
| | | | | | | | | | GTGATGATCAAATTTGGGCGCCAACTTCTGATAACAAGCATGAG |
| | | | | | | | | | CTGGAAACTTTGAGGAAGGAGTTGGATGCTGCATTGGCTAAAGT |
| | | | | | | | | | TGCTGAAT (SEQ ID NO: 17) |
| common_4485 | NC_044377.1 | 72948533 | 5.369889 | A | G* | 0.11 | 0.24 | 0.18 | CGGCTACGCTTTCGCCGGGGATAGCTCTCTTCCCGCGCCGGT |
| | | | | | | | | | CATATTTTCCGGCGTTCCTCCGGCGACAACCGCTACTGCCACC |
| | | | | | | | | | GCTTGGTCACCTTCCTTATCGTCTGCTCTCTACAAAGTCGATG |
| | | | | | | | | | GGTGGGGCGCACCTTACTTCGCCGTCAACTCTTCCGGCAACG |
| | | | | | | | | | TCGCCGTTCGCCCTCACGGCGCTGGAACTTT[G/A]GCGCACCA |
| | | | | | | | | | GGAGATTGATTTGCTGAAAATTGTAAAGAAGGTTTCGGATCCG |
| | | | | | | | | | AAATCTAAGGGCGGGTTAGGTTTGCCCCTTCCGCTCGTTATTC |
| | | | | | | | | | GGCTTCCTGATGTGCTTAAGAACCGCCTTGAGTCTCTCCAGGC |
| | | | | | | | | | GGCGTTCGATTTCGCAATCCAGTCGCAGGACTATGAAAACCAT |
| | | | | | | | | | TACCAGGGTGTTTACCCTGTG (SEQ ID NO: 1) |
| common_4446 | NC_044377.1 | 68780117 | 5.323884 | A | T* | 0.026 | 0.23 | 0.09 | TTTGTTAATTGTTTCATTTTTCTGAAATGCAAATGATGATATTTTT |
| | | | | | | | | | ATGAAGGTTGGAAGAATGGCGGGTCAGTTTGCCAAGCCCAGA |
| | | | | | | | | | TCAGATTCATTTGAGGATAGGGATGGAGTGAAGCTTCCTAGTT |
| | | | | | | | | | ACAGAGGTGACAATGTGAATGGTGATGCATTTGATGAAAAATC |
| | | | | | | | | | GAGAATTCCAGACCCTAATCGAATGAT[T/A]AGAGCCTATACAC |
| | | | | | | | | | AATCTGCTGCAACTTTGAATCTTTTGAGGGCATTTGCTACTGGA |
| | | | | | | | | | GGTTATGCTGCTATGCAGAGAGTGACCCACTGGAATCTAGATT |
| | | | | | | | | | TCACTGATCACAGTGAGCAGGGGGATAGGTAAACTTTTATTGT |
| | | | | | | | | | TCTTTTCTTCTTACTTGAAATTTTGAATGTTTATTTTCCATAATGA |
| | | | | | | | | | ATAGGATTGAAG (SEQ ID NO: 18) |
| common_4514 | NC_044377.1 | 75675432 | 5.184746 | A* | G | 0.31 | 0.11 | 0.18 | CACTTTAAGTTATAAATTACGTTGTAACTAAAAGTAAAAATCTTT |
| | | | | | | | | | GTAGTGTAAATTTATATATATTTACCTCGGAGACCATGTCATTG |
| | | | | | | | | | AAAACTCTTCCCATTATTTCCATCTTATGTTTAGGATCATTACTC |
| | | | | | | | | | ATAACACTTCCCATCATGGTTAGTACCATACTCCCACCACTTAC |
| | | | | | | | | | TATTTCCTCTGAACGACATCTTA[A/G]AAAGCTTGTGAAATCCTC |
| | | | | | | | | | TTGAAATTGATTCAAGTAAGCTTTCACAACTGAATTAGGGCTTC |
| | | | | | | | | | TTTTTGTAATATAAATGTTGCCTTTGTTAAGTGCTTCTCCTGTTT |
| | | | | | | | | | CCCCAACCAAACCACTTGGAACCTTCAAAACAACATTAATTCAC |
| | | | | | | | | | ATAATTAATAAACTAAAACCTTATAAAAAGAAAGGGTAAATTTCA |
| | | | | | | | | | ATTTT (SEQ ID NO: 19) |

### Example 3

### Genome-Wide Association Studies (GWA) of Purple Color in an F2 Population in Cannabis

To confirm the ability to monitor the transmissibility of the purple color through monitoring SNP markers associated with this trait in the next generations, to identify additional SNP markers associated with purple color in cannabis, and to identify candidate genes that may be involved in the presence or absence of purple color, the inventors generated an F2 population designated GID: 21 002 035 0000 from the selfing of a progeny from parents GID: 20 000 104 0000, known to be stable for the appearance of purple color in the whole plant, and GID: 20 000 072 0000, known to rarely display purple color in the whole plant.

They assessed purple color visually of the whole plant from F2 population GID: 21 002 035 0000 consisting of 137 individuals. At the time of harvest, genotypes were visually assessed for the presence of purple in the whole plant, the areas on leaf, stem, and flower.

Plants were assessed for purple color in the whole plant with a score from 1 to 9, where 1 indicates a completely green plant and 9 a completely purple plant. A total of 41 (28,87% of total population) plants were scored less than 5 (predominantly green), while 101 (71,12% of total population) plants were scored greater than or equal to 5 (more purple). This indicates a dominant allele controlling purple color in the whole plant and the flower and that the trait is transmissible.

DNA was extracted from about 70 mg of leaf discs from all the plants evaluated using an adapted “sbeadex kit” with magnetic beads by LGC Genomics, automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified are provided in Table 5. The library pool was loaded onto Ion 550 chips with Ion Chef and sequenced with Ion GeneStudio S5 Plus according to the manufacturer's instructions (Ion 550™ Kit from Thermo Fisher Scientific).

From a population of 137 individuals, a genome-wide association analysis (GWAS) was performed to detect significant associations between genotypic information derived from targeted resequencing of the custom SNP marker panel described above and the appearance of purple color in the whole plant. Plants were assigned a score from 1 to 9, where 1 indicates a completely green plant and 9 a completely purple plant.

The genotypic matrix was filtered for SNPs having more than 30% missing values within the population and a minor allele frequency lower than 5%. This resulted in 4212 SNP markers after filtering. The GWAS was performed using GAPIT version 3 (Wang and Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The Blink model performed the best by the inventors' evaluation and was used for the analysis. SNPs surpassing an LOD (−log₁₀(p-value)) value of 5 were considered to have a significant association with trait variation.

Here, SNPs significantly associated with purple color in the whole plant were found exclusively on chromosome NC_044377.1, listed in Table 3. The inventors show that the presence of the indicative homozygous allele is strongly associated with purple color in this segregating population. The SNPs identified in Table 3 are useful in predicting the presence or absence of purple color in the whole plant. The inventors show that the heterozygous state of the allele associates with purple color, though less so than the homozygous state. The homozygous state of the reference allele is clearly associated with plants that are not purple.

From SNP marker “common_4519” (Table 3), showing the highest association purple color in the F2 population, a constant decrease in LOD values can be observed for neighboring increasingly distant markers, showing an erosion of linkage disequilibrium caused by recombination. This observation shows the marker panel used in this study can be used to monitor linkage decay across the genome and for determining the QTL with high confidence.

TABLE 3
SNPs associated with the purple color trait in a whole plant, F2 population 21 002 035
0000 on Chromosome NC_044377.1. The presence of the purple color trait is predicted by the
occurrence of the indicative allele (marked with *). The positions of the SNPs are provided with
reference to the CS10 reference genome as described herein. “Homo_1” denotes the average
phenotypic value associated with homozygous allele 1 based on a score from 1-9, as described
in the text, where 1 indicates a green plant and 9 indicates a purple plant, “Homo_2” denotes the
average phenotypic value associated with homozygous allele based on a score from 1-9, as
described in the text, where 1 indicates a green plant and 9 indicates a purple plant, and “Hetero”
denotes the average phenotypic value associated with heterozygous based on a score from 1-9,
as described in the text, where 1 indicates a green plant and 9 indicates a purple plant. BP refers
to the nucleotide position of the SNP.

| | | | Allele_ | Allele_ | Homo_ | Homo_ | | |
|---|---|---|---|---|---|---|---|---|
| SNP | BP | LOD | 1 | 2 | 1 | 2 | Hetero | Context Sequence |
| common_4519 | 76201790 | 10.96014 | A | G* | 2.18 | 7.4 | 6.74 | CGATCACTTCGTAGATGCATCCTCCCACAAGGTAGCACAATTGTAGAAAGTGCTAAA |
| | | | | | | | | TCATGCTTTATTCCATTTGTTCTTTTTGTCTTCTCTTTTTGCTTAATCGAACGATGTTG |
| | | | | | | | | TGAACTTGTAGGGTTGTCAAATTCGAAGGGAGAGCGCACATGGAGAGAGCGTTTGT |
| | | | | | | | | TGCAACAATGTGAGGGCCCTGTTTGATGA[A/G]CTCCCAACTCCACACCTAATTGTG |
| | | | | | | | | GAGATCACACCATTCCCTGAAGGGCCTCTCACTGAAAAAGATTACACCAAAGCTGAG |
| | | | | | | | | AAATTGGAGAGGGTACTTAGAACTGGCCCGAACGTTTGATTCTTCTCTCGAGTTAAA |
| | | | | | | | | TCATCGCTGTCTCTCGTTAGAACTACAGCTTAATTGTATGTATGTTTTGAGCCTTGTA |
| | | | | | | | | CATAT (SEQ ID NO: 15) |
| common_4525 | 76757669 | 10.61882 | A | C* | 2.38 | 7.76 | 6.72 | ACAAGTCTTATCTAAACGAAAAGCTCCACAACTATGGAGATGAACACTACCAGAGAA |
| | | | | | | | | GCAAAACATAACAGTTAGCTTCAGTTCATAATTTATTTACAGTCTATCATACACTGTTC |
| | | | | | | | | TACAAGTGCTTGCTGAGGATGGATTCGACAACACCTTGTGCAATAGGATGATAACAA |
| | | | | | | | | TCGCGAGCCTCTGCAAACACCCTTTTCG[C/A]GAAAATCTTCTCTTCTTCCTTCCCGT |
| | | | | | | | | TCCCTTGAACGAGCGCAGTGTAGAGTGGACGAAGGTATTTCATCCTCCCAACTTCTT |
| | | | | | | | | TGAGAGTTTTCTCCACCTCGCCGTAGTAGTCTCTGCACTTGGCGGTAATGGCTAGCT |
| | | | | | | | | GCAGAAACCCCACCTTCACCTCGTAATCTTTTGATTCTGAGAGCCTGTAGCGCTGGT |
| | | | | | | | | CCAA (SEQ ID NO: 20) |
| common_4500 | 74383124 | 9.883966 | A* | C | 7.35 | 2.37 | 6.86 | TCGACTTCAATGAGAAATCTCAACCCCTGTGGTAAGAATTTTGTGTACTTTTTTAAAA |
| | | | | | | | | ATTGAATTTTTAAAATTTCCCAGTTCGCTGAATATTTGTATTAACTGTGTCTAATTTTTC |
| | | | | | | | | ATACTAGACATTGAAAGAATGGTGTCTTTGAAGGGAATGGTTATCCGTTGCAGTTCA |
| | | | | | | | | ATAATTCCGGAAATCAGAGAAGCAAT[C/A]TTTAGATGTCTTGTGTGCGGTTACTACT |
| | | | | | | | | CTGAACCTGTTGCTGTAGAGAAAGGTAATTAGTTAGGATACCATTCGCATGGGCTAG |
| | | | | | | | | TTTTTTCTTATCTATATCACAATGTCATTCTAAATTATTTTCCCTTTTCAGGACGGATAA |
| | | | | | | | | CTGAACCAACAAGATGCTTGAAGGAAGAATGCCAAGCAAGAAACTCCATGACACTT |
| | | | | | | | | (SEQ ID NO: 13) |
| common_4487 | 73084792 | 9.515762 | A* | G | 7.41 | 2.75 | 6.64 | CTGTCTCATATATTCAACCTCGTCACAAACATTTATTGTTTCGTGGGTCGTTTTCCAC |
| | | | | | | | | TTCTTCATATCTACACAATAATCAATCGGGGCTTCCTTTGTTTTTTGTTATGTGAAAAT |
| | | | | | | | | GGTTGCAGAAGTAGAAGAAGAATGAGAGAGTTAAGATTAGAAAGAAAGAAGAATGC |
| | | | | | | | | GAACCCTTTGTGACGTTTGTGAGAAAGC[T/C]GCCGCGATCCTCTTCTGCGCCGCCG |
| | | | | | | | | ACGAGGCTGCTCTATGCAGTTCCTGCGATGACAAGGTCTTTCACTAATTTAAGCTTTT |
| | | | | | | | | AATAATTTTTAATTTAATTTACTAACTTCAAATCATTGGTATGAAGTTATTTCAATTCAA |
| | | | | | | | | ATATTATTTATATGAATTGGGTTTTGTATTTTGTTATCTGCTGGAATTTTCATTGTTT |
| | | | | | | | | (SEQ ID NO: 21) |
| GBScompat_ | 73090006 | 9.271999 | A | G* | 2.55 | 7.32 | 6.76 | CTTCAGCCCAAACCTTTTGAGCAACCTCAAAAGTCCTATCAATACCATGCTCAC |
| rare_165 | | | | | | | | CATCAAGTAAGATCTCTGGCTCAACAATTGGGACCAAACCATTGTCCTAAACAT |
| | | | | | | | | AACAAAACATAATCAATTTTCACAAACACAAGTACAAAAATCAAGATTAGCTTAG |
| | | | | | | | | TCTTGTCTGATTAGTTATCTTACTTGGGAGATAGCAGC[A/G]TAGCGGGCTAGA |
| | | | | | | | | CCCCAAGCGGCTTCCTTCACAGCCAGAGCTGATGGGCCATTGGGAATGCTCA |
| | | | | | | | | CAACAGTACGCCTGTTTAAATAATGTAATTTTGTGTCAAATACCGATTAAAGAGC |
| | | | | | | | | ATTGCTATAAGGCATTATAGTGTCCAACATCACTATTAGGCACTACGGGTGCTT |
| | | | | | | | | TTTAACATTTCTCAACCAGACAAAAGTA (SEQ ID NO: 12) |
| GBScompat_ | 72805722 | 9.055252 | A * | C | 7.27 | 2.67 | 6.6 | AGCAGAGAAAATTGAAAAAATGACGGAAAGGAAAAAGAAGAGGGAAGCAACAC |
| rare_164 | | | | | | | | CAGTATGCTTTGTGGCCACAGGGAAGTGAACAAGGTATAAATCCAGATAATCTA |
| | | | | | | | | ACTGAAGCTTCTTCAGACTATTCTTACAGGCCTCAAGGACATGTCCATGGTCAG |
| | | | | | | | | AATTCCAAAGCTGCAATCAAGACAGGTAAAGAAAAAGAAT[A/C]ATAGTCTGGCT |
| | | | | | | | | GGACATACAGAATATACATATTATTCGATCAGTTCGAAAATAAACTAACCTTAGT |
| | | | | | | | | GGTAACAAAGAGATCTTCTCTCTTAACAAGCCCTGTCTGAAATGCCTCAGAAAG |
| | | | | | | | | TGCCTCCCCAACTTCTGTTTCATTCCTGTAATCAGCTGCAACCAACACCAGCTA |
| | | | | | | | | GTCAAACATTACATTGTAGCAACTCAA (SEQ ID NO: 22) |
| common_4513 | 75673037 | 8.673232 | A | C* | 2.15 | 7.42 | 6.63 | ATAATGGGTTTTGATTTGTTCTGATACTCAGATTGAACCAAAGAGAAGGCCATC |
| | | | | | | | | TCCGAGTTACCTTGAGAAAGTTCAGAGTGAAATCAGTGCCAACATGAGAGGAG |
| | | | | | | | | TATTGGTGGATTGGTTGGTGGAAGTTGCAGAGGAGTACAAACTTGGTTCAGAG |
| | | | | | | | | ACTCTTTACCTATCTGTTTCTTATATTGATCGATTCTTGTC[A/C]TTGAACACCAT |
| | | | | | | | | TGCCAGGAATAAGCTTCAGTTATTGGGTGTTTCTTCTTTGCTCGTTGCCTCGTA |
| | | | | | | | | AGATTCTAACCCTTTTGAACTAATGTTAATGAAGATGATATGTTGAATTTGATTT |
| | | | | | | | | GTTTATTCATATAAAGTTTTGATTTTTATCTTTGGTTTCACTTGTTTAGAAAGTAT |
| | | | | | | | | GAAGAGATTAATCCTCCTAATGTGG (SEQ ID NO: 23) |
| common_4522 | 76434921 | 8.634652 | A | G* | 2.56 | 7.47 | 6.33 | GACCTGGCGCCGGATCGATCCGGTTCCGAAATACGCGGACGGGTTGCCTATG |
| | | | | | | | | TTCTGCCACGTGGCGGGTTGTGAAGGTAAGCTGGTTGTAATGGGCGGTTGGG |
| | | | | | | | | ATCCGAAAAGCTACGGACCCGTTTCGGATGTTTTCGTTTTCGATTTCGCTAAGA |
| | | | | | | | | ATCGGTGGAGCGAAGCCAAGGCCATGCCGGGTAAGAGGTCGTT[C/T]TTTGCG |
| | | | | | | | | GTCGGGTCTTATTCGGGTCGGATCTATGTTGCGGGGGGCACGATGAGAATAA |
| | | | | | | | | GAACGCGTTGAGTTCTGCTTGGGTTTACGATGTGAGTCTGGACGAGTGGAGCG |
| | | | | | | | | AGTTGGCTCAGATGAGTCAGGGCCGTGACGAGTGCGAAGGCGTGGTGTTAAA |
| | | | | | | | | CGGTGAGTTTTGGGTTGTGAGTGGGTACGGCACCGAC (SEQ ID NO: 24) |
| common_4465 | 70904920 | 8.431373 | A * | G | 7.31 | 2.89 | 6.66 | ATAGTGAGTTCTTTAATGAAACGTACCCATTCCATTATTTGGTTTAGTTTCATTT |
| | | | | | | | | AATATTATTGAAAGTGGAACATCAAATTTAAGAAGATCCATATTTTAATTGGTTT |
| | | | | | | | | TGGAAATTGTACATGAGGAATGATTAAGAAGACGTAGGCAACCAATTATAGAT |
| | | | | | | | | GCACATTAATTTAGTTCATTTAGATCCTAATCCATGCA[A/G]CTGGCCCAATTAT |
| | | | | | | | | TGGAGATCTCATGGTTTTGGGTATCTCAAATCTAAGCATAGCCACTGGCTCAG |
| | | | | | | | | TCGGAGATCTCATGGTTCTTGCAATATCTTTGAGAATAAATTTCTGAATTTTCC |
| | | | | | | | | CAGTTGAGGTCTTGGGAAGTTCATCTCTGAACACAACAACTTTAGGAACCATA |
| | | | | | | | | TAATGAGGCAACTTCTCTCTACAATACT (SEQ ID NO: 25) |
| common_4475 | 71862900 | 8.379069 | A * | G | 7.2 | 3.15 | 6.76 | AGGTTACATCATCAATAAATGAATAGCACTACCAATAGAAGGAAAGAGTGATC |
| | | | | | | | | ATGTGACATATTTCATATATGGCTCTTGAAACTAACCAAACACCACTCAAAAAA |
| | | | | | | | | AATCTACATTTCTCTCCACATATCACACCTTCCCCTACTCATATTTTCTTCTATA |
| | | | | | | | | TATGTCACTAAATCATAAACTCCCACATACAAGTACTCT[A/G]CTCAAGCCTTAT |
| | | | | | | | | TACAAACACTCCTCTCTTCTACAAAAACCTCTCTGTTTCAAACATGGCTATAAG |
| | | | | | | | | ATTACATGGAATGTTGAATGCTAAGCACCTCCTACGCCGCTCGAGTTTCTCTC |
| | | | | | | | | AAAACCATGGAGTTTCAACACCCATCGACGTTCCCAAAGGCTATCTCGCGGT |
| | | | | | | | | GTATGTTGGAGAAGAACACAAGAAGAGACA (SEQ ID NO: 26) |
| common_4414 | 64950520 | 8.173088 | A | G* | NA | 6.8 | 5.66 | CCTATTCAAGATATCATGGCCTTCAGTTCGGCAATTCATAATTCTTCGAAAGAC |
| | | | | | | | | TTCTCTTAGTTGACGCTCCAATGGATCCAATCCATTTCGAAAACTTTCACAAAC |
| | | | | | | | | CGCAGCTACCTCATGCACCCTTTGCTCTACCTCCTCCTTTTGCTCTTCTGTCA |
| | | | | | | | | AGGGAAACTGAACAGAATCCACCAAGTCAGTCATGTGGCG[T/C]GCACATCTT |
| | | | | | | | | TCAACCTGATAAATCTCCCTCAACAATCCATTGGTGTTTCGATTCTCTCTCTTT |
| | | | | | | | | TTGGATTCTTCCAAAATCCGATTGAGAAGTGACATCAAAGGCGTTCCCCATGA |
| | | | | | | | | GAAATTGCGGGGCACTGAGAAATGGACATTAAGACCGCGATCCTGACATGGG |
| | | | | | | | | ATTGCAGCCACAAGAGTCCACAAAACAAACATG (SEQ ID NO: 27) |
| common_4463 | 70588691 | 8.14361 | A | G* | 2.89 | 7.32 | 6.58 | TGGTCAGTGTGAATTCCACTATTGTGTGAATTGTGTCACTTGCACATCCAGGC |
| | | | | | | | | AAAGAGTAAAATATGAATATCATGATCATCCTCTTTTTTTTCTCGACAAAATTCA |
| | | | | | | | | CACTGGTCTGGTGGAATGTGATGATTATGATTCTTATTGTAAGAATCCAATTTT |
| | | | | | | | | (GGCTGAATGTGACGAATTCAAGTATACCAATTCATCTGT[G/A]TTTGGTTGTGA |
| | | | | | | | | TCAAGAATGTAATTTTAAAGTTCATTTGCTATGTGGTCCATTACCTAGCACTCT |
| | | | | | | | | TAAATATGAATATCACATACATCCTCTTATTTTGTTTGATTTTGTTCTCAACAAT |
| | | | | | | | | GATTATGGAGACTTTTACTGTGATATTTGTGAAATGAAAAGAGATCCACGAATA |
| | | | | | | | | CGTGTATACTTTTGTGAAGATTGCAAC (SEQ ID NO: 28) |
| common_4504 | 74637355 | 8.033517 | A* | G | 7.48 | 2.36 | 6.66 | TTAACTTAATGATCATATAGATAGTAATAAACTATTAATTAATTAATTTT |
| | | | | | | | | GCTGGGGATGAGAATGGTGGCCAGGTAGCTTTTCCTTGATCTTTTCC |
| | | | | | | | | ATAACAGTTTTCTTCTCGTGAAGAGGAGTAGGATCATGAGTAGTAGC |
| | | | | | | | | AGTTGGTTCGTCCTTGTGTTTTCCAGTAAGTTTCTCTTTTATTTTTGTT |
| | | | | | | | | CCTAAACC[C/T]TTCTTCTTCCTTCTCCCTCCAGCTCCATCATCCTCTG |
| | | | | | | | | ACTGCATCCACACCCATTTTATAATTATTATATATATTAATCATTCATTT |
| | | | | | | | | TTAATATATATAATTACATTATATTATTATTTTAATATTTTAAAATGTAAA |
| | | | | | | | | TAAAAAATTTAGTGATTCAAATTATAATTTTTTATATTATATGTTGATAG |
| | | | | | | | | CAATTTTGTTATT (SEQ ID NO: 29) |
| rare_547 | 69138750 | 7.817705 | A | C* | 3.22 | 7.34 | 6.66 | AAGACAAAAGAATAAGCTAATTAAGAAGCTAAAGAAAGAGATCAACTC |
| | | | | | | | | TACTTTGAATTTGTTGTTTCAGAACAATATGACTACAAATCGATGGTG |
| | | | | | | | | GGCTGGTAATGTGGCCATGAGAGGAGTGGATTCCATGTCTTCACCAC |
| | | | | | | | | CACCACCACCTCCATTGCTTCAGCTCAGAAACACAGATGAAGATCCC |
| | | | | | | | | AACAAAGATGA[C/A]GACCAAGACAGTGGCAACGGCGGCGACGACGA |
| | | | | | | | | CGACCCCAATTCAACCGGCCACGAAAGCTTCGGACTAGGAGGAAGC |
| | | | | | | | | AGCAGCAACAACCGCAGGCCACGTGGCAGACCCCCAGGTTCCAAGA |
| | | | | | | | | ACAAGCCAAAGCCTCCGGTAGTGATAACAAAAGAGAGCCCCAATGCT |
| | | | | | | | | CTAAGAAGCCACGTTTTGGAAATCAGTAGC (SEQ ID NO: 30) |
| GBScompat_ | 71021186 | 7.666785 | A | G* | 3 | 7.39 | 6.58 | TATCTCCAAATACCCATCTGGGTGCATAACTCCCACATCGCCCGTGT |
| common_869 | | | | | | | | AAAACCATCCATCCTCCTTTATAGCTTTAGCAGTAGCCAGTTCATCTT |
| | | | | | | | | TAAGGTAGCCCAACATGACGCAATTACCTCTTAACACCACTTCCCCC |
| | | | | | | | | ATTGTGAAACCATCTCTCTTCACACTCAACCCCGAATTCGGATCAACA |
| | | | | | | | | ACATCAACTTC[A/G]GTCATTGCTGCTGAATTCACTCCTTGGCGCGCC |
| | | | | | | | | TTTAGCCGGGCGCGCTCAGTAGCTGGAAGAAGATTCCACTTCTTCTT |
| | | | | | | | | CCAAGCGCAAGACACAACGAGTCCGCACACTTCTGTCAACCCGTAG |
| | | | | | | | | CCATGACTAACTTTAAAACCGAGCGACTCAGTTCGGTGTAGAACCGC |
| | | | | | | | | CGCGGGAGGTGGAGCTCCTGCGGTAAGG (SEQ ID NO: 31) |
| common_4462 | 70351069 | 7.629437 | G* | A | 7.32 | 2.89 | 6.63 | TGTTTATATTATGAGGAAAAGAGGTTTGGTCCCAAGCTTGGTGTCATA |
| | | | | | | | | TAATTCTATTGTACATGGACTTAGCAAGGAAGGAGGCTGTATGAGGG |
| | | | | | | | | CTTATCAGTTGTTAGAAGAAGGCATTGAATTCGGATATCGACCATCTG |
| | | | | | | | | AGCATACTTACAAGGTCTTAGTAGAAGGTCTTTGCCGAGAAAATGAC |
| | | | | | | | | CTTCACAAGGC[G/A]AAATTTGTCTTTCATGTTATGCTCAACAAGGAAG |
| | | | | | | | | GAGTTGAGAGAACTAGATTTTATAACATATATCTTAGAGCTCTTTGTTT |
| | | | | | | | | TATGGATAATGCTGCTACTGAGCTTTTGAATACACTTGTTTCTATGCT |
| | | | | | | | | CCAAACTCAGTGTCAACCTGATGTGATCACTCTCAATACCATTATTAA |
| | | | | | | | | AGGGTTTTGTAAGATGGGGAGA (SEQ ID NO: 32) |
| common_4486 | 72986295 | 7.529719 | C | G* | 2.57 | 7.25 | 6.72 | TCAATTTTGTTACTGTTATCTTTAATGCTTTCAATTGAATAAAATTC |
| | | | | | | | | AAGGGGTTAATATTATAGCAATTCTGCAGCGTCTCTACATATTGA |
| | | | | | | | | TTATCCTATGCTGTTTAAGCTTCAGAATGACTCTGCAAATAAAGT |
| | | | | | | | | CTCACACTGTGGTGTCTTGGAGTTTATTGCTGAGGAAGGCATGA |
| | | | | | | | | TATATATGCCTTATTGGGTA[C/G]GGATTGGTTTTTTCTGCGAAAT |
| | | | | | | | | ATTCACCTGTACAGAGTTTTCTGGTCCATTTTTATCTAGCTTTTGA |
| | | | | | | | | CCCATGGTGATTTGCACTCATTAGTGTTTTGTTGGTTCTGTTTAT |
| | | | | | | | | CTATTAGATGATGCAAAATATGCTCTTAGGAGAGGGAGACTTTGT |
| | | | | | | | | GCGTGTGAAGAATGTGACTCTTCTGAAGGGGACATATGTTAAA |
| | | | | | | | | (SEQ ID NO: 33) |
| common_4472 | 71630810 | 7.443011 | A* | G | 7.28 | 2.96 | 6.72 | ATTCATGAAAGAAAATTTCTAATAATATTCATTTGTATCATTCTTG |
| | | | | | | | | GGGACTACATTTAGTTGTTAATCCCCAAGGAAAAATTAATTACAA |
| | | | | | | | | GGTATATGGCATTCTCTGCCTAACAAATTGTAACTGTGAGTTTTG |
| | | | | | | | | TTAGCTGTTATCTTCTACTGGTGTCTAGACTCATTTGATTAGGAG |
| | | | | | | | | CTTAAGTGAGAAATAAGATC[G/A]ATGAAGGCATCTTCTGTGCAT |
| | | | | | | | | GGAATTGTAAGAGCTCCCATTGGATGATCGAACCCGAATTCTTCT |
| | | | | | | | | TCAGCTTGATTTAGAAAGTCTTGGAATGAAAGCTGGTTCAAGTAT |
| | | | | | | | | GCCACAGGGATCACAAATCTCTTCATTATGCTCTCATCGCGAACA |
| | | | | | | | | TAAACCGCCAAAAAGCCTTTAGGGATATCTTTTGTGCTTGAGAAA |
| | | | | | | | | (SEQ ID NO: 34) |
| common_4517 | 75907527 | 7.402701 | A* | C | 7.43 | 2.33 | 6.7 | CACAGCATCTCCATTAGAAGGATTAAACTTATGAGTACTAGCATG |
| | | | | | | | | AGCATACCCACTTGCAAACCTAAAAAGACCACTTCCACCAATCAC |
| | | | | | | | | AGGCATTTCTCTAACCTTATCAAACACTTGGTTTCTACCAAGAAT |
| | | | | | | | | GGTGAGAGTGCTCCCATTGTATTTCCCTTGAGTTATATGAAAATT |
| | | | | | | | | CATAGCCATAATTAGGGCAAT[C/A]TCTTCTTGTGAAGCTAATCCA |
| | | | | | | | | TAAAACCCTTGAGCTTTTCCTAGCAACTTTGAGCTTACTTCTGGC |
| | | | | | | | | CCTTCTGTCAATGGATTGTCGATCATGCTAACCGCCCCGAACCC |
| | | | | | | | | ACTTTTCGATGCATTGGCCGGTGGTTGGATTATTGCCATCGCGC |
| | | | | | | | | TAGGGTTTTTGCCGCTGTATATGTCGTGCCAATAGAACCGAAAG |
| | | | | | | | | TGG (SEQ ID NO: 35) |
| common_4474 | 71780297 | 7.270959 | A* | G | 7.33 | 2.91 | 6.62 | ACGTAATGCTTTGATGTATTTTCTGTCATCTAAATTCGGAATACCATCTAGAAT |
| | | | | | | | | ATTTTGACAGTCTTCCACATTACATGAACTAGTGATATTGCTTGATTTTCCATT |
| | | | | | | | | GATTATCTTCTCCCTTGTATCAATATACTTACCCATACTTTCACTTTGTACAAG |
| | | | | | | | | TGATGCCTCAGTTTTAGCAGCTATACTTTTTGCAAGTTC[C/T]TCCATCACATG |
| | | | | | | | | TTCAATGATAGAATTAGTCCCTTTGTTCTTTTTCTCCTTTGGTCTTGGTGCATC |
| | | | | | | | | AAGTGGAACAGAAGTAGCTCGACGACGTACACCAGTAACTTCTTCTTCCTCT |
| | | | | | | | | CCAAAATTTTCACCATCGCATTCAATACCATCATCAACATGTGCTCCAGTGTC |
| | | | | | | | | AATATAATGCCGCTCTTGTTCATGTTCCTCA (SEQ ID NO: 36) |
| common_4432 | 66679345 | 7.165743 | A* | G | 7.27 | 3.29 | 6.63 | AGTAGTAGCAACGGTGAATTGACAACAAAAGTTTGAAGTCATTAACTACCCCA |
| | | | | | | | | TGGCCATCAAAAACTCACCCTCACATACAACCTCACCTCCAACATATGCTTTC |
| | | | | | | | | CCCTCCATTTTCGCTATTCCAAAGCGTTTCTGCAGCTTTGTGAGTGTCATTCT |
| | | | | | | | | CATAACTAAAGTGTCACCAGCAATCACTGGTTTCCGGAATCT[A/G]ACTTTGTC |
| | | | | | | | | GATTCCAGCAAAAAAGAAGTTCTCCCGAGAACCTCCCACTTCTGGTTGCAGC |
| | | | | | | | | ATCACCAAACCACCAACCTGAGCCATTGCCTAGATCCAACAAAATTGAATATG |
| | | | | | | | | TTGTTTTGTTAAAATTACATTACATGATGACTAAATAGATCAGAATATTTCCTA |
| | | | | | | | | CAAGGACAGCAAAAAAATTCATTTTTCTAGGAAC (SEQ ID NO: 37) |
| common_4452 | 69326994 | 7.113516 | A* | G | 7.33 | 3.18 | 6.62 | GAGTGCCGTATATTTGTATTTAAACATTAGTCAACCAATATGATCAAATGTATA |
| | | | | | | | | TATACGGTTACATATTACGCATATATATGAATCAAAGTTATATTACTTTCTCAAT |
| | | | | | | | | ATGATCAAAGTTGTGATTTTGTTGGTGCTAGCCACACTCATTAATCAAGTATG |
| | | | | | | | | GAGTAGTACTACAAGTAATAATTATTGCATAGAGAAGGA[G/A]AGACAAGCTC |
| | | | | | | | | TTCTCAACTTGAAGAAAGGCTTTGTCGATGATGGCAATCGTCTATCCTCATGG |
| | | | | | | | | ACAAGTAGTAGCCGTGATTGTTGTGCATGGAGAGGTATCAGGTGCGATAACT |
| | | | | | | | | CAAAAACTCATCGTCATATTATCGCTCTTGATCTTAAATCTGATGACAACAATC |
| | | | | | | | | ATAATTATTTGGGTGGTGAAATTGGTCCTTCT (SEQ ID NO: 8) |

### Example 4

### Validation of Purple Color Markers in Cannabis

The inventors identified SNP markers that are associated with purple color in whole cannabis plants. To validate the usefulness of the SNP markers identified, they evaluated their effectiveness in predicting the presence of purple color in cannabis plants in a different F₂ population of cannabis plants. This F2 population, designated GID: 21 002 046 0000, was made from the selfing of a progeny of parents GID: 20 000 006 0000, known to not display the appearance of purple color in the whole plant, and GID: 20 000 083 0000, known to display purple color in the whole plant.

Purple color was visually assessed of the whole plant from F2 population GID: 21 002 046 0000 consisting of 113 individuals. At the time of harvest, plants were visually assessed for the presence of purple in the whole plant, the areas on leaf, stem, and flower. Plants were assessed for purple color in the whole plant with a score from 1 to 9, where 1 indicates a completely green plant and 9 a completely purple plant. A total of 30 (26.54% of total population) plants were scored less than 5 (more green), while 83 (73.45% of total population) plants were scored greater than or equal to 5 (more purple). This indicates a dominant allele controlling purple color in the whole plant and the flower and that the trait is transmissible.

DNA was extracted from about 70 mg of leaf discs from all the plants evaluated using an adapted “sbeadex kit” with magnetic beads by LGC Genomics, automated on a KingFisher Flex with 96 Deep-Well Head by Thermo Fisher Scientific.

The extracted DNA served as a template for the subsequent library preparation for sequencing. The library pools were prepared according to the manufacturer's instructions (AgriSeq™ HTS Library Kit-96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified are provided in Table 5. The library pool was loaded onto Ion 550 chips with Ion Chef and sequenced with Ion GeneStudio S5 Plus according to the manufacturer's instructions (Ion 550™ Kit from Thermo Fisher Scientific).

From a population of 113 individuals, a genome-wide association analysis (GWAS) was performed to detect significant associations between genotypic information derived from targeted resequencing of the custom SNP marker panel described above and the appearance of purple color in the whole plant. Plants were assigned a score from 1 to 9, where 1 indicates a completely green plant and 9 a completely purple plant.

The genotypic matrix was filtered for SNPs having more than 30% missing values within the population and a minor allele frequency lower than 5%. This resulted in 4015 SNP markers after filtering. The GWAS was performed using GAPIT version 3 (Wang and Zhang, 2021) with four statistical models: General Linear Model (GLM), Mixed Linear Model (MLM), FarmCPU and Blink (model=c (“GLM”, “MLM”, “FarmCPU”, “Blink”). A quantile-quantile plot (QQ plot) was used to evaluate the statistical models. The Blink model performed the best by the inventors' evaluation and was used for the analysis. SNPs surpassing an LOD (−log₁₀(p-value)) value of 5 were considered to have a significant association with trait variation.

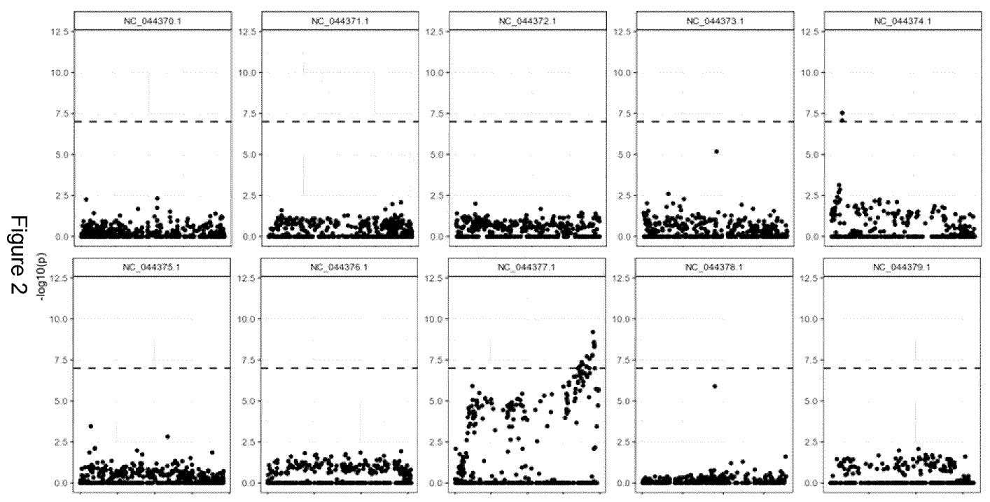

Here the inventors identified additional SNPs that significantly associate with purple color in the whole plant on chromosome NC_044377.1 and NC_044374.1 listed in Table 4 (FIG. 2).

The inventors then looked specifically at the three SNPs on chromosome NC_044377.1 identified in Example 3 from population GID: 21 002 035 0000 with the highest LOD scores: “common_4519”, “common_4525” and “common_4500” (Table 3). They found that in the F2 population, designated GID: 21 002 046 0000, these SNP markers were strongly linked to the gene and/or causative SNP underlying the appearance of purple color in cannabis, based on their LOD scores. These SNP markers can be used to predict of the presence or absence of purple color in the whole plant, including the flower. In the case of SNPs “common_4519”, “common_4525”, and “common_4500”, when homozygous for the indicative allele, in these cases at allele 2, on average plants were found to have a score above 7, indicating a highly purple plant. For these three SNPs, when heterozygous for the indicative allele, on average plants were found to have a score under 7, indicating that these were slightly less purple than the homozygous case. When, alternatively, the plants were homozygous for allele 1, on average the purple score was below 3.65, indicating a plant that is not purple. This shows that it is possible to produce a plant with purple color from a cross between a non-purple plant and a plant that is homozygous or heterozygous for the alleles associated with purple color, without relying on the appearance of purple color to determine selection. Through the use of the provided markers it is possible to determine the allele state of the SNPs for the purple trait in order to identify the presence of the trait in the absence of the appearance of purple color to aid selection and identification of plants with SNPs associated with the purple trait.

When considering the SNPs associated with purple color found in the GWAS from the two F2 populations, a well-defined QTL on chromosome NC_044377.1 can be defined (Table 4, FIG. 2). This QTL is well defined by the SNPs “GBScompat_common_864” and “GBScompat_common_879” at reference positions 68717484 to 77040783 on chromosome NC_044377.1. The SNP markers, as well as the entire region that make up the QTL, are linked to the gene and/or causative SNP underlying the appearance of purple color in cannabis as demonstrated by the linkage decay observed to a level under the LOD threshold of 5.

A second QTL associated with purple color can also be defined based on this experiment on NC_044374.1 based on the SNP markers “common_2448”, “GBScompat_common_473”, and “GBScompat_rare_86”. This QTL is defined by the genomic region linked to these SNP markers and can be considered to be centered at position 6600328 on NC_044374.1 with reference to the CS10 genome of Cannabis Sativa.

TABLE 4
Validation of Purple Color in Whole Plant, F2 population 21 002 046 0000 showing the
SNPs associated with the purple color trait. The presence of the purple color trait is predicted by
the occurrence of the indicative allele (marked with *). The positions of the SNPs are provided
with reference to the CS10 reference genome as described herein. “Homo_1” denotes the
average phenotypic value associated with homozygous allele 1 based on a score from 1-9, as
described in the text, where 1 indicates a green plant and 9 indicates a purple plant, “Homo_2”
denotes the average phenotypic value associated with homozygous allele based on a score from
1-9, as described in the text, where 1 indicates a green plant and 9 indicates a purple plant and
“Hetero” denotes the average phenotypic value associated with heterozygous based on a score
from 1-9, as described in the text, where 1 indicates a green plant and 9 indicates a purple plant.
BP refers to the nucleotide position of the SNP.

| | | | | Allele_ | Allele_ | Homo_ | Homo_ | | |
|---|---|---|---|---|---|---|---|---|---|
| SNP | Chromosome | BP | LOD | 1 | 2 | 1 | 2 | Hetero | Context Sequence |
| common_4519 | NC_044377.1 | 76201790 | 9.201071 | A | G* | 3.34 | 7.27 | 6.8 | CGATCACTTCGTAGATGCATCCTCCCACAAGGTAGCACAATTG |
| | | | | | | | | | TAGAAAGTGCTAAATCATGCTTTATTCCATTTGTTCTTTTTGTCT |
| | | | | | | | | | TCTCTTTTTGCTTAATCGAACGATGTTGTGAACTTGTAGGGTTG |
| | | | | | | | | | TCAAATTCGAAGGGAGAGCGCACATGGAGAGAGCGTTTGTTG |
| | | | | | | | | | CAACAATGTGAGGGCCCTGTTTGATGA[A/G]CTCCCAACTCCAC |
| | | | | | | | | | ACCTAATTGTGGAGATCACACCATTCCCTGAAGGGCCTCTCAC |
| | | | | | | | | | TGAAAAAGATTACACCAAAGCTGAGAAATTGGAGAGGGTACTT |
| | | | | | | | | | AGAACTGGCCCGAACGTTTGATTCTTCTCTCGAGTTAAATCATC |
| | | | | | | | | | GCTGTCTCTCGTTAGAACTACAGCTTAATTGTATGTATGTTTTG |
| | | | | | | | | | AGCCTTGTACATAT (SEQ ID NO: 15) |
| common_4526 | NC_044377.1 | 76803154 | 8.582297 | A | G* | 3.61 | 7.38 | 6.76 | TTTATATTACCTACATGAAATCAGACAACAACAGCATATCGGGG |
| | | | | | | | | | GCTTCACTCATGGAGATGGAGACCTACTACCATTTGGTTCATCT |
| | | | | | | | | | GGGTAGCGATTTGGACTTCGGCTAAGGCTTCTCTTTGGGCTTC |
| | | | | | | | | | GGCTTCTGCTTCGACTATGGCTTGGACTTCTAGCTCCACTTGG |
| | | | | | | | | | ACCCCTAGCTGGGCTTCGACTTGGGCT[T/C]CTTGATCCATTAT |
| | | | | | | | | | ATGGTGGAGAACGAGAGTACGACCTGTCTCGGCTGTACCTTCT |
| | | | | | | | | | CTCCTGTGGAGACACAGATCTGGCCCGAAGAGGGAGTACGAA |
| | | | | | | | | | AATTAATGTTTAAACATTGAAATATATTAAATGCATGTTTTCTCC |
| | | | | | | | | | TATTGCTAAAGATCCCACATTTTTAATGCTGACTAGAGAAGTTG |
| | | | | | | | | | AAAAGATATACTTG (SEQ ID NO: 38) |
| common_4525 | NC_044377.1 | 76757669 | 8.534953 | A | C* | 3.62 | 7.22 | 6.72 | TTTATATTACCTACATGAAATCAGACAACAACAGCATATCGGGG |
| | | | | | | | | | GCTTCACTCATGGAGATGGAGACCTACTACCATTTGGTTCATCT |
| | | | | | | | | | GGGTAGCGATTTGGACTTCGGCTAAGGCTTCTCTTTGGGCTTC |
| | | | | | | | | | GGCTTCTGCTTCGACTATGGCTTGGACTTCTAGCTCCACTTGG |
| | | | | | | | | | ACCCCTAGCTGGGCTTCGACTTGGGCT[T/C]CTTGATCCATTAT |
| | | | | | | | | | ATGGTGGAGAACGAGAGTACGACCTGTCTCGGCTGTACCTTCT |
| | | | | | | | | | CTCCTGTGGAGACACAGATCTGGCCCGAAGAGGGAGTACGAA |
| | | | | | | | | | AATTAATGTTTAAACATTGAAATATATTAAATGCATGTTTTCTCC |
| | | | | | | | | | TATTGCTAAAGATCCCACATTTTTAATGCTGACTAGAGAAGTTG |
| | | | | | | | | | AAAAGATATACTTG (SEQ ID NO: 20) |
| GBScompat_ | NC_044377.1 | 77040783 | 8.407491 | A* | T | 7.33 | 3.82 | 6.8 | CAAAATTATTTTAGTAAAGGTTCCTTCTTTAGATAAGAAAGAA |
| common_879 | | | | | | | | | CAAAATATGGCCATTGTATCATCAACCATTTCAAATAGGAAAC |
| | | | | | | | | | AATTAGATTCTCAGTACTCTAAACCTTTGATTCCTGATCCTGC |
| | | | | | | | | | TAGTACCAAACACATGGCTGCTCTTAAGCTTCAAAAGGTCTA |
| | | | | | | | | | CAAAAGCTTCCGAACTAGACGAAAGTTAGC[T/A]GATTGTGCT |
| | | | | | | | | | GTTCTTGTAGAGCAGAGTTGGTATGTAACTAATCTTTTATGTT |
| | | | | | | | | | CAATTCTAGTCTTTGTTGTGTTGTGTTGTTCTGTCATGTTCTG |
| | | | | | | | | | ATATACAATTTTTTGCATCAATTTTAGGTGGAAGCTCTTAGATT |
| | | | | | | | | | TTGCTGAACTCAAGAGGAGTTCGATATCTTTTTTCGATATTGA |
| | | | | | | | | | GAAGCATGAAACCGCGATT (SEQ ID NO: 39) |
| common_4528 | NC_044377.1 | 76959457 | 8.296782 | A* | G | 7.22 | 3.82 | 6.64 | AAGTAAATAATATTTACATAAGTGAATTAGAATGAAGTAATAA |
| | | | | | | | | | GCAATAAAGTGCCACAAACTCCAACAAATCCAACTACAAATC |
| | | | | | | | | | CCACTCCAATTCCCATATAAAGCCATGACATGTTAAGCCACC |
| | | | | | | | | | CTTCATCATATTCTTCAGCATCACTTGGATCATGAATATCTTTA |
| | | | | | | | | | GGGCTATTTGGTGTCTCATCTCCAAGACAT[A/G]AATTGTTTA |
| | | | | | | | | | GTGGAAGTCCGCACAATCCATCATTATCAATGTATATCGAAG |
| | | | | | | | | | CATTAAAACTTTGCAATTGAGTACCGATAGGAATTCTTCCAGA |
| | | | | | | | | | CAATTTGTTACTTGACAAATTCAAAAAAGATAGAGAAGATATA |
| | | | | | | | | | CTTGCCAAGCTTGTTGGAATGACACTAGAAAGCTTGTTATGG |
| | | | | | | | | | GATAAATCTAGAGAATCCAACT (SEQ ID NO: 40) |
| common_4518 | NC_044377.1 | 75977377 | 7.797678 | A* | G | 7.3 | 3.17 | 6.87 | TTTTATTTCTCTCACTTTTTTTGCATACTCTTTTCTTTCCATTCT |
| | | | | | | | | | TCTCGATCGTGCTGAAATACCTAATAAGACAGTGACACAGCA |
| | | | | | | | | | TGGCATGACACAATTAATGGGAGCGGTTGCCTTTGCACAACA |
| | | | | | | | | | ACTCCTCCTCTTCCACCTCCACTCTGCTGATCATATGGGACC |
| | | | | | | | | | AGAGGGACAATATCACTTGCTACTCCAGCT[C/T]GTGATTCTT |
| | | | | | | | | | GTCTCTCTGGTCACATCTCTAATGGGAATAGGGCTACCGAAG |
| | | | | | | | | | AGTTTCTTAGTGAGTTTTGTTAGGTCTCTTAGCATTTTGTTTCA |
| | | | | | | | | | AGGGGTTTGGCTTATGGTGATGGGGTTTATGTTATGGACACC |
| | | | | | | | | | ATCCTTGATTTCCAAAGGGTGTTTCATGCACTATGAGGAAGG |
| | | | | | | | | | TCATCATGTGGTGAGATGCTCA (SEQ ID NO: 41) |
| common_4517 | NC_044377.1 | 75907527 | 7.761131 | A* | C | 7.38 | 3.13 | 6.86 | CACAGCATCTCCATTAGAAGGATTAAACTTATGAGTACTAGCATG |
| | | | | | | | | | AGCATACCCACTTGCAAACCTAAAAAGACCACTTCCACCAATCAC |
| | | | | | | | | | AGGCATTTCTCTAACCTTATCAAACACTTGGTTTCTACCAAGAAT |
| | | | | | | | | | GGTGAGAGTGCTCCCATTGTATTTCCCTTGAGTTATATGAAAATT |
| | | | | | | | | | CATAGCCATAATTAGGGCAAT[C/A]TCTTCTTGTGAAGCTAATCCA |
| | | | | | | | | | TAAAACCCTTGAGCTTTTCCTAGCAACTTTGAGCTTACTTCTGGC |
| | | | | | | | | | CCTTCTGTCAATGGATTGTCGATCATGCTAACCGCCCCGAACCC |
| | | | | | | | | | ACTTTTCGATGCATTGGCCGGTGGTTGGATTATTGCCATCGCGC |
| | | | | | | | | | TAGGGTTTTTGCCGCTGTATATGTCGTGCCAATAGAACCGAAAG |
| | | | | | | | | | TGG (SEQ ID NO: 35) |
| GBScompat_ | NC_044377.1 | 72805722 | 7.710366 | A* | C | 7.15 | 3.77 | 6.83 | AGCAGAGAAAATTGAAAAAATGACGGAAAGGAAAAAGAAGAGGG |
| rare_164 | | | | | | | | | AAGCAACACCAGTATGCTTTGTGGCCACAGGGAAGTGAACAAGG |
| | | | | | | | | | TATAAATCCAGATAATCTAACTGAAGCTTCTTCAGACTATTCTTAC |
| | | | | | | | | | AGGCCTCAAGGACATGTCCATGGTCAGAATTCCAAAGCTGCAAT |
| | | | | | | | | | CAAGACAGGTAAAGAAAAAGAAT[A/C]ATAGTCTGGCTGGACATA |
| | | | | | | | | | CAGAATATACATATTATTCGATCAGTTCGAAAATAAACTAACCTTA |
| | | | | | | | | | GTGGTAACAAAGAGATCTTCTCTCTTAACAAGCCCTGTCTGAAAT |
| | | | | | | | | | GCCTCAGAAAGTGCCTCCCCAACTTCTGTTTCATTCCTGTAATCA |
| | | | | | | | | | GCTGCAACCAACACCAGCTAGTCAAACATTACATTGTAGCAACTC |
| | | | | | | | | | AA (SEQ ID NO: 22) |
| common_4500 | NC_044377.1 | 74383124 | 7.570859 | A* | C | 7.16 | 3.57 | 6.91 | TCGACTTCAATGAGAAATCTCAACCCCTGTGGTAAGAATTTTGTG |
| | | | | | | | | | TACTTTTTTAAAAATTGAATTTTTAAAATTTCCCAGTTCGCTGAATA |
| | | | | | | | | | TTTGTATTAACTGTGTCTAATTTTTCATACTAGACATTGAAAGAAT |
| | | | | | | | | | GGTGTCTTTGAAGGGAATGGTTATCCGTTGCAGTTCAATAATTCC |
| | | | | | | | | | GGAAATCAGAGAAGCAAT[C/A]TTTAGATGTCTTGTGTGCGGTTA |
| | | | | | | | | | CTACTCTGAACCTGTTGCTGTAGAGAAAGGTAATTAGTTAGGATA |
| | | | | | | | | | CCATTCGCATGGGCTAGTTTTTTCTTATCTATATCACAATGTCATT |
| | | | | | | | | | CTAAATTATTTTCCCTTTTCAGGACGGATAACTGAACCAACAAGA |
| | | | | | | | | | TGCTTGAAGGAAGAATGCCAAGCAAGAAACTCCATGACACTT |
| | | | | | | | | | (SEQ ID NO: 13) |
| common_2448 | NC_044374.1 | 6600147 | 7.544585 | A* | C | 7.22 | 3.85 | 6.75 | GAATGTGTTGGAATCCCATTCTTAATTTGATATTGAAATTTTT |
| | | | | | | | | | ATTGCTGTTTTCCAGTCTTGGCATGAAAGACTCTTGGGGGC |
| | | | | | | | | | CTTTGAAGGCTTTGGCTGTAGCTAGCATTATAAATGGCATT |
| | | | | | | | | | GGTGATATACTCCTGTGCAGAGTTTTTAGCTATGGCATTGC |
| | | | | | | | | | TGGTGCAGCATGGGCGACGATGGCATCACAGGTGC[T/G]C |
| | | | | | | | | | CAGATGAACATTTTGCTCCACTGTCTTTCCAGATTATATATT |
| | | | | | | | | | TCACTTTAGTTCTTATTAATTTCCGAGAAATATCCTAAATGA |
| | | | | | | | | | GTTTGTTTTCTTTCATACTGCCACTAACACAAATAGTATTAG |
| | | | | | | | | | GTTGTTGCAGGGTATATGATGGTTGAAAATCTGAACAAGAA |
| | | | | | | | | | AGGTTACAATGCTTATGCTCTCTCCATTCCCTC (SEQ ID |
| | | | | | | | | | NO: 42) |
| GBScompat_ | NC_044374.1 | 6600328 | 7.528498 | A* | T | 7.23 | 3.69 | 6.58 | GACGATGGCATCACAGGTGCTCCAGATGAACATTTTGCTCC |
| common_473 | | | | | | | | | ACTGTCTTTCCAGATTATATATTTCACTTTAGTTCTTATTAAT |
| | | | | | | | | | TTCCGAGAAATATCCTAAATGAGTTTGTTTTCTTTCATACTG |
| | | | | | | | | | CCACTAACACAAATAGTATTAGGTTGTTGCAGGGTATATGA |
| | | | | | | | | | TGGTTGAAAATCTGAACAAGAAAGGTTACAATGC[T/A]TATG |
| | | | | | | | | | CTCTCTCCATTCCCTCACCGAAAGAACTTATCGCTATACTTG |
| | | | | | | | | | AGCTTGCTGCTCCGGTATTCATCACTATGACTTCTAAGGTA |
| | | | | | | | | | AATATTACTCAGTTTTCCTTGAGCTTGGCTATAATCTTTCCTT |
| | | | | | | | | | AGTTTTCCTTCAAAAACTAAGGTGTTTATATCCTTAGGTGGC |
| | | | | | | | | | ATTCTATAGTCTCCTCATATATTTTGCTA (SEQ ID NO: 43) |
| common_4459 | NC_044377.1 | 69980258 | 7.36557 | A* | C | 7.42 | 3.77 | 6.61 | TAGGCAAAGCATGTCAAGACTGGGGCTTCTTTATGGTAATT |
| | | | | | | | | | ATTAATTAAGCTTAATTAATATTAATACCTCTAAAACAATATT |
| | | | | | | | | | GATGTCATATTTGAAATATTGCTATTCTTTTATGATTAATATA |
| | | | | | | | | | TAGGTGATCAATCATGGTGTGGCAGAGAGATTAATGAGTGA |
| | | | | | | | | | AGTTTTAGAAGGGTGTAGAGGTTTTTTTGATCT[T/G]AGTGA |
| | | | | | | | | | AGAAGAGAAGCTTGTGTTTAAGGGTACACATGTTATGGACA |
| | | | | | | | | | CAATTAGGTATGGTACAAGCTTCAATGCATCGGTAGAGAAA |
| | | | | | | | | | GCTTTGTATTGGAGAGATTATCTTAAGGTTCTTGTTCCTCAG |
| | | | | | | | | | CACCATCCTCATCATTTTCATTTCCCTAATAACCCATCTGGG |
| | | | | | | | | | TTCAGGTAATATTATTCACACATAAAATTT (SEQ ID NO: 44) |
| common_4463 | NC_044377.1 | 70588691 | 7.235412 | A | G* | 3.86 | 7.38 | 6.66 | TGGTCAGTGTGAATTCCACTATTGTGTGAATTGTGTCACT |
| | | | | | | | | | TGCACATCCAGGCAAAGAGTAAAATATGAATATCATGATC |
| | | | | | | | | | ATCCTCTTTTTTTTCTCGACAAAATTCACACTGGTCTGGTG |
| | | | | | | | | | GAATGTGATGATTATGATTCTTATTGTAAGAATCCAATTTT |
| | | | | | | | | | GGCTGAATGTGACGAATTCAAGTATACCAATTCATCTGT[G/A] |
| | | | | | | | | | TTTGGTTGTGATCAAGAATGTAATTTTAAAGTTCATTTG |
| | | | | | | | | | CTATGTGGTCCATTACCTAGCACTCTTAAATATGAATATCA |
| | | | | | | | | | CATACATCCTCTTATTTTGTTTGATTTTGTTCTCAACAATGA |
| | | | | | | | | | TTATGGAGACTTTTACTGTGATATTTGTGAAATGAAAAGAG |
| | | | | | | | | | ATCCACGAATACGTGTATACTTTTGTGAAGATTGCAAC |
| | | | | | | | | | (SEQ ID NO: 28) |
| common_4451 | NC_044377.1 | 69163028 | 7.124024 | A* | C | 7.18 | 3.72 | 6.64 | AAATGGCACCGCATCCGAAACAACAAATATCCCAATCAAA |
| | | | | | | | | | CGAAAGAACTCACTTATTGCCTTATGACACCGTCTTGCTT |
| | | | | | | | | | CATCCCCACTCTGATCATCGCTACCCGATGCTCCGAAATA |
| | | | | | | | | | ACGCTTTCCAGCCACCATTCTCACCACCATGTTAAGAGTT |
| | | | | | | | | | AAATCTTCTAACCAACGACTCAACTCAACTACAACTTTACA |
| | | | | | | | | | [A/C]GACTTGTAGAGCTCTCTAATCCCTACTTCAACCTCTG |
| | | | | | | | | | AAATCCTCACTTGCTTCAACATCTCTAGACGGCGGTTAGA |
| | | | | | | | | | GAGGAGTTCTAACGTGGCGATCTTCCTCATTTCGCGCCAA |
| | | | | | | | | | AAAGGGCTATAAGGTGCGAAACCAAAGACTGCGTAGTTG |
| | | | | | | | | | TAGCCCATGTGCTTGGCTGCCACGGTTGTAGGGCGCGAG |
| | | | | | | | | | GCCAGC (SEQ ID NO: 10) |
| common_4483 | NC_044377.1 | 72698292 | 7.106316 | A | G* | 3.81 | 7.25 | 6.74 | CGACTTCTTTGCCGAAGATAAGAAAAACCCTGATAGCAGT |
| | | | | | | | | | TTCGATGAATATTTCTACGATGATGACGAAAAGCCTCGCG |
| | | | | | | | | | AGGAGTGTGGTGTTGTGGGTATTTATGGCGACTCAGAGG |
| | | | | | | | | | CCTCTCGGCTCTGTTACTTGGCTCTTCACGCTCTCCAACA |
| | | | | | | | | | TCGTGGTCAAGAAGGGGCTGGAATTGTTGCTGTGAAAAA |
| | | | | | | | | | CGA[C/T]GTTCTTCAATCCGTTACAGGCGTTGGACTTGTCT |
| | | | | | | | | | CTGAAGTCTTTAGCCATTCAAAGCTCGATCAATTGCCTGG |
| | | | | | | | | | AGATTTGGCTATTGGCCATGTACGGTACTCTACTGCTGGG |
| | | | | | | | | | TCTTCTATGCTTAAAAATGTTCAACCTTTTGTTGCAGGGTA |
| | | | | | | | | | TAGATTTGGTTCAGTTGGTGTTGCACACAATGGCAATTTG |
| | | | | | | | | | GTAAAT (SEQ ID NO: 45) |
| GBScompat_ | NC_044374.1 | 6600352 | 7.082896 | A | G* | 3.74 | 7.25 | 6.5 | GATGAACATTTTGCTCCACTGTCTTTCCAGATTATATATTTCACT |
| rare_86 | | | | | | | | | TTAGTTCTTATTAATTTCCGAGAAATATCCTAAATGAGTTTGTTT |
| | | | | | | | | | TCTTTCATACTGCCACTAACACAAATAGTATTAGGTTGTTGCAG |
| | | | | | | | | | GGTATATGATGGTTGAAAATCTGAACAAGAAAGGTTACAATGCT |
| | | | | | | | | | TATGCTCTCTCCATTCCCTCACC[G/A]AAAGAACTTATCGCTATA |
| | | | | | | | | | CTTGAGCTTGCTGCTCCGGTATTCATCACTATGACTTCTAAGGT |
| | | | | | | | | | AAATATTACTCAGTTTTCCTTGAGCTTGGCTATAATCTTTCCTTA |
| | | | | | | | | | GTTTTCCTTCAAAAACTAAGGTGTTTATATCCTTAGGTGGCATT |
| | | | | | | | | | CTATAGTCTCCTCATATATTTTGCTACATCCATGGGCACAATCA |
| | | | | | | | | | GCATGG (SEQ ID NO: 46) |
| GBScompat_ | NC_044377.1 | 68717484 | 7.054155 | A | G* | 3.77 | 7.35 | 6.64 | TAGTGGGTTAGGAACTGGGAGCAAAGGCCTAAGCGGCGGACA |
| common_864 | | | | | | | | | GAAGAGGAGAGTGAGCATCTGCATAGAGATTCTCACTCGTCCA |
| | | | | | | | | | AAGCTTCTTTTCCTTGACGAGCCCACTAGTGGGCTCGACAGTG |
| | | | | | | | | | CTGCTTCATACTATGTGATGAGCAGCATTGCGTCGTTGGATATT |
| | | | | | | | | | CAGAGTCGGAGGGGTGGTGGGGCCGGTGG[C/T]CGGAGGACT |
| | | | | | | | | | GTGGTGGCTTCCATCCACCAGCCCAGTTCCGAAGTGTTTCAGC |
| | | | | | | | | | TTTTTAATACTCTTTGCCTTCTTTCTGCTGGTAAAATTGTGTATT |
| | | | | | | | | | TTGGTCCTGCTAGTGCAGCTAATGAGGTATTTTCAGGTTTTTTT |
| | | | | | | | | | TAAACGTATTTAAATTATAAATTACAATACATAAAGGAATAATAA |
| | | | | | | | | | TATTTGTACTAATTA (SEQ ID NO: 47) |

TABLE 5
Targeted sequencing primers (5′ to 3′) for the SNPs identified in Tables 1 to 4, as
described in Examples 1 to 4.

| SNP | Forward Primer | Reverse Primer |
|---|---|---|
| common_1535 | GCAAATGAGTTGGTGAGCCA | GCCAATGCAGCATCCAACTC |
| | (SEQ ID NO: 48) | (SEQ ID NO: 49) |
| common_2032 | GGGGTGATGTGAATGAGTGC | CCTGGGATGCTGTGTGTTCT |
| | (SEQ ID NO: 50) | (SEQ ID NO: 51) |
| common_2262 | AAATTAAGAGCAGTTTCACGTGT | TGCTCGAATCTGCTACATTGC |
| | (SEQ ID NO: 52) | (SEQ ID NO: 53) |
| common_2448 | AAGACTCTTGGGGGCCTTTG | CCATCATATACCCTGCAACAACC |
| | (SEQ ID NO: 54) | (SEQ ID NO: 55) |
| common_4054 | TTACCCATCGTGGCTGACTG | CGCAACTAACGGCAACCAAA |
| | (SEQ ID NO: 56) | (SEQ ID NO: 57) |
| common_4414 | TTCACAAACCGCAGCTACCT | TGGACTCTTGTGGCTGCAAT |
| | (SEQ ID NO: 58) | (SEQ ID NO: 59) |
| common_4432 | AGCAACGGTGAATTGACAACA | AATGGCTCAGGTTGGTGGTT |
| | (SEQ ID NO: 60) | (SEQ ID NO: 61) |
| common_4446 | TTGGAAGAATGGCGGGTCAG | TATCCCCCTGCTCACTGTGA |
| | (SEQ ID NO: 62) | (SEQ ID NO: 63) |
| common_4451 | ACACCGTCTTGCTTCATCCC | TGGGCTACAACTACGCAGTC |
| | (SEQ ID NO: 64) | (SEQ ID NO: 65) |
| common_4452 | TGTTGGTGCTAGCCACACTC | AGGACCAATTTCACCACCCA |
| | (SEQ ID NO: 66) | (SEQ ID NO: 67) |
| common_4459 | AAGCATGTCAAGACTGGGGC | ACCGATGCATTGAAGCTTGT |
| | (SEQ ID NO: 68) | (SEQ ID NO: 69) |
| common_4462 | GAAGGAGGCTGTATGAGGGC | GGTTGACACTGAGTTTGGAGC |
| | (SEQ ID NO: 70) | (SEQ ID NO: 71) |
| common_4465 | AAGGGTAAGGGAGAGTGGCA | AGCTCGACTAATGGGGATGA |
| | (SEQ ID NO: 72) | (SEQ ID NO: 73) |
| common_4472 | GCATTCTCTGCCTAACAAATTGT | CCTAAAGGCTTTTTGGCGGT |
| | (SEQ ID NO: 74) | (SEQ ID NO: 75) |
| common_4474 | GTGATGCCTCAGTTTTAGCAGC | GGAACATGAACAAGAGCGGC |
| | (SEQ ID NO: 76) | (SEQ ID NO: 77) |
| common_4475 | TCCACATATCACACCTTCCCC | TCTCCAACATACACCGCGAG |
| | (SEQ ID NO: 78) | (SEQ ID NO: 79) |
| common_4483 | TTATGGCGACTCAGAGGCCT | ACCAAATTGCCATTGTGTGCA |
| | (SEQ ID NO: 80) | (SEQ ID NO: 81) |
| common_4485 | TATTTTCCGGCGTTCCTCCG | GGAGAGACTCAAGGCGGTTC |
| | (SEQ ID NO: 82) | (SEQ ID NO: 83) |
| common_4486 | CAATTCTGCAGCGTCTCTACA | CACGCACAAAGTCTCCCTCT |
| | (SEQ ID NO: 84) | (SEQ ID NO: 85) |
| common_4487 | TGTTTCGTGGGTCGTTTTCC | ACCTTGTCATCGCAGGAACT |
| | (SEQ ID NO: 86) | (SEQ ID NO: 87) |
| common_4499 | CAGATAGGACACTCTGAACAAGC | TCAGGCTCGAATGCAAGAGA |
| | (SEQ ID NO: 88) | (SEQ ID NO: 89) |
| common_4500 | GAGAAATCTCAACCCCTGTGGT | AACTAGCCCATGCGAATGGT |
| | (SEQ ID NO: 90) | (SEQ ID NO: 91) |
| common_4502 | TCAGTGGAGTCTCTGGTGACA | GGCTAGCCCGCTTTATCACA |
| | (SEQ ID NO: 92) | (SEQ ID NO: 93) |
| common_4504 | TGCTGGGGATGAGAATGGTG | ATGGGTGTGGATGCAGTCAG |
| | (SEQ ID NO: 94) | (SEQ ID NO: 95) |
| common_4513 | AGAGAAGGCCATCTCCGAGT | GAATCTTACGAGGCAACGAGC |
| | (SEQ ID NO: 96) | (SEQ ID NO: 97) |
| common_4514 | ACCTCGGAGACCATGTCATTG | GGTTTGGTTGGGGAAACAGG |
| | (SEQ ID NO: 98) | (SEQ ID NO: 99) |
| common_4517 | GCATGAGCATACCCACTTGC | CGGCCAATGCATCGAAAAGT |
| | (SEQ ID NO: 100) | (SEQ ID NO: 101) |
| common_4518 | TTAATGGGAGCGGTTGCCTT | GCATCTCACCACATGATGACC |
| | (SEQ ID NO: 102) | (SEQ ID NO: 103) |
| common_4519 | GATGCATCCTCCCACAAGGT | CGGGCCAGTTCTAAGTACCC |
| | (SEQ ID NO: 104) | (SEQ ID NO: 105) |
| common_4522 | GGACGGGTTGCCTATGTTCT | ACTCATCTGAGCCAACTCGC |
| | (SEQ ID NO: 106) | (SEQ ID NO: 107) |
| common_4525 | CAAGTGCTTGCTGAGGATGG | GCGCTACAGGCTCTCAGAAT |
| | (SEQ ID NO: 108) | (SEQ ID NO: 109) |
| common_4526 | CAACAGCATATCGGGGGCTT | CTCTTCGGGCCAGATCTGTG |
| | (SEQ ID NO: 110) | (SEQ ID NO: 111) |
| common_4528 | ACAAATCCCACTCCAATTCCCA | GTCATTCCAACAAGCTTGGCA |
| | (SEQ ID NO: 112) | (SEQ ID NO: 113) |
| common_4599 | ACGAAAAAGAGGCGCTTCTTG | AGGTCCAGTTAAGCTGTTGTAAGT |
| | (SEQ ID NO: 114) | (SEQ ID NO: 115) |
| common_5220 | ATTGATGGTGGTGATGGGCA | TCCGAGGACGACTTTCTAGGT |
| | (SEQ ID NO: 116) | (SEQ ID NO: 117) |
| common_816 | CCTGATCATGGGCAGCATCA | TTAGAAACAGCCTGGTGGGG |
| | (SEQ ID NO: 118) | (SEQ ID NO: 119) |
| GBScompat_ | GCATCACAGGTGCTCCAGAT | AGTGATGAATACCGGAGCAGC |
| common_473 | (SEQ ID NO: 120) | (SEQ ID NO: 121) |
| GBScompat_ | GTGGGTTAGGAACTGGGAGC | AAACACTTCGGAACTGGGCT |
| common_864 | (SEQ ID NO: 122) | (SEQ ID NO: 123) |
| GBScompat_ | ACATCGCCCGTGTAAAACCA | TCATGGCTACGGGTTGACAG |
| common_869 | (SEQ ID NO: 124) | (SEQ ID NO: 125) |
| GBScompat_ | ACCAAACACATGGCTGCTCT | ATCGCGGTTTCATGCTTCTC |
| common_879 | (SEQ ID NO: 126) | (SEQ ID NO: 127) |
| GBScompat_ | GGGAAGCAACACCAGTATGC | AACAGAAGTTGGGGAGGCAC |
| rare_164 | (SEQ ID NO: 128) | (SEQ ID NO: 129) |
| GBScompat_ | CAGCCCAAACCTTTTGAGCA | CAGGCGTACTGTTGTGAGCA |
| rare_165 | (SEQ ID NO: 130) | (SEQ ID NO: 131) |
| GBScompat_ | ACAAACTGCTGCCTCTGTATCT | GTGCCAGCCATTCTCAAAGC |
| rare_2 | (SEQ ID NO: 132) | (SEQ ID NO: 133) |
| GBScompat_ | TGCTCCACTGTCTTTCCAGA | GCCAAGCTCAAGGAAAACTGA |
| rare_86 | (SEQ ID NO: 134) | (SEQ ID NO: 135) |
| rare_547 | TCGATGGTGGGCTGGTAATG | TGGCTTCTTAGAGCATTGGGG |
| | (SEQ ID NO: 136) | (SEQ ID NO: 137) |
| common_4448 | TGATCAGCGAAGAAAGGCCA | AGCATCACGGCTATGACACC |
| | (SEQ ID NO: 138) | (SEQ ID NO: 139) |
| common_4463 | TCACTTGCACATCCAGGCAA | AGTCTCCATAATCATTGTTGAGAACA |
| | (SEQ ID NO: 140) | (SEQ ID NO: 141) |

### Example 5

### Gene Identification

There are presently no known genes identified in Cannabis that have been shown to regulate color in flowers or throughout the whole plant. Genes that regulate flower color through the biosynthesis of anthocyanins or through their transcriptional regulation have been described and characterized in several plant species. The inventors considered genes that regulate anthocyanin levels as being the best candidate genes for controlling the appearance of purple in cannabis color observed. They next sought to identify putative genes that could encode proteins that may be responsible for the accumulation of anthocyanins in the total plant and in the flower. Using the findings of the association studies they identified candidate genes at the QTLs identified.

At the QTL found on chromosome NC_044373.1 based on the SNP “common_2262” at position 80922439, the inventors looked at a 2 mB region centering on this SNP for putative candidate genes. Genes and annotation of the CS10 reference genome (GCF_900626175.1) were retrieved from NCBI. Scans for known amino acid domains were performed using hmmer (v3.1, with the option −E 1e-5) with the Pfam database (v33, Finn et al). Gene description, related KEGG pathways and GO terms were identified using Pannzer (v2, Toronen et al.) using default settings and manually inspected. The inventors identified two genes with gene ID LOC115712034 and LOC115712567 listed in Table 6. Both are annotated as acyl-transferase family proteins. A BLAST search of the amino acid sequences encoded by these genes of all Arabidopsis thaliana proteins returned an HXXXD-type acyl-transferase family protein as the closest homolog. Acyl-transferases, like the two identified, may be involved in transferring acyl-groups to the sugar moieties of anthocyanins affecting the purple color of plant tissue through the stability of the anthocyanin, causing them to either accumulate or dissipate.

Based on the results of the association study for purple color from the F2 population 21 002 046 0000 the inventors identified a QTL on NC_044374.1 marked by the three SNPs: “common_2448”, “GBScompat_common_473”, and “GBScompat_rare_86”. They looked for putative candidates in the region of this QTL by manual inspection of an annotated gene list for chromosome NC_044374.1 from the Cannabis sativa CS10 genome. The inventors identified a candidate gene within 0.1 Mb that is annotated to encode an acyl transferase family protein, with gene ID LOC115716241 listed in Table 6. A BLAST search of the amino acid sequences encoded by these genes against all Arabidopsis thaliana proteins returned an HXXXD-type acyl-transferase family protein as the closest homolog. Acyltransferases, like the two identified, may be involved in transferring acyl-groups to the sugar moieties of anthocyanins affecting the purple color of plant tissue through the stability of the anthocyanin, causing them to either accumulate or dissipate.

From the QTL found on NC_044377.1 between position 64950520-77040783 the inventors searched for genes that may encode proteins involved in the biosynthesis or transcriptional regulation of anthocyanins from an annotated gene list for this region of NC_044377.1 from the Cannabis sativa CS10 genome. Upon inspection of this genomic region and BLAST analysis of putative candidates they identified five candidate genes LOC115695758, LOC115725215, LOC115695887, LOC115695872, LOC115695871 listed in Table 6. The gene IDs LOC115695758, LOC115725215, and LOC115695887 encode putative MYB Transcription factors. MYB Transcription factors, in other plant species, act as regulators of secondary metabolism, including positively and negatively regulating anthocyanin biosynthesis.

The inventors identified that candidate gene LOC115695758, mRNA XM_03062284, encodes the protein XP_030478701.1 that is homologous to a MYB domain containing transcription factor. Inspection of the protein sequence with SMART: Simple Modular Architecture Research Tool (Letunic et al. (2017) Nucleic Acids Res; Letunic et al. (2020) Nucleic Acids Res) identified two SANT Repressor domains at positions 14-64 and 67-115. The inventors determined that mutagenesis to functionally alter the proteins activity and or approaches to disrupt transcription of the protein would result in the manifestation of purple color in cannabis plants primarily in, but not restricted to, flowers.

The inventors identified that the candidate gene LOC115695887, mRNA XM_030622986, encodes the protein XP_030478846, which was predicted to contain a domain homologous to a GT1 domain. GT1 domains are DNA binding domains that are components of transcriptional regulators, particularly activators. The transcription of this gene may be repressed in plants lacking anthocyanin pigment. The inventors determined that mutagenesis to functionally alter the proteins activity and or approaches to enhance transcription of the protein would result in the manifestation of purple color in cannabis plants primarily in, but not restricted to, flowers.

The inventors further identified that the candidate gene LOC115725215, mRNA XM_030654653, encodes the protein XP_030510513, which was predicted to contain a domain homologous to a GT1 domain. GT1 domains are DNA binding domains components of transcriptional regulators, particularly activators. The transcription of this gene may be repressed in plants lacking anthocyanin pigment. The inventors determined that mutagenesis to functionally alter the proteins activity and or approaches to enhance transcription of the protein would result in the manifestation of purple color in cannabis plants primarily in, but not restricted to, flowers.

The inventors also identified two genes LOC115695871 and LOC115695872 that are annotated as encoding putative anthocyanidin 3-O-glucosyltransferase. The inventors identified the candidate gene LOC115695871, mRNA XM_030622963.1, encoding the protein XP_030478823.1. XP_030478823.1 was predicted to encode an anthocyanidin 3-O-glucosyltransferase 2. The inventors identified the candidate gene LOC115695872, mRNA XM_030622964, encoding the protein XP_030478824.1. XP_030478824.1 was predicted to encode an anthocyanidin 3-O-glucosyltransferase 2 with 47% identity to XP_030478823.1. Glucosyltransferase proteins transfer the sugar moiety to anthocyanidin. Anthocyadins are stabilized by the addition of a sugar moiety. This suggests a mechanism for the regulation of purple color in cannabis whereby the loss or gain of function of this protein would affect the accumulation of anthocyanins in plant tissue.

TABLE 6
Gene list of candidate genes identified. The gene ID is provided
with reference to the publicly available CS10 genome.

| | Start | End | | | |
|---|---|---|---|---|---|
| Chromosome | Position | Position | Gene ID | Protein ID | Description |
| NC_044373.1 | 79836159 | 79837767 | LOC115712034 | XP_030496100.1 | HXXXD-type acyl- |
| | | | | | transferase family protein |
| NC_044373.1 | 79968804 | 79970536 | LOC115712567 | XP_030496724.1 | HXXXD-type acyl- |
| | | | | | transferase family protein |
| NC_044374.1 | 6676610 | 6680038 | LOC115716241 | XP_030500856.1 | HXXXD-type acyl- |
| | | | | | transferase family protein |
| NC_044377.1 | 75409856 | 75419127 | LOC115695758 | XP_030478701.1 | MYB domain TF, 2 SANT |
| | | | | | Domains |
| NC_044377.1 | 75894244 | 75898321 | LOC115725215 | XP_030510513.1 | MYB domain TF |
| NC_044377.1 | 76275921 | 76280609 | LOC115695887 | XP_030478846.1 | MYB domain TF |
| NC_044377.1 | 76403822 | 76405684 | LOC115695872 | XP_030478824.1 | anthocyanidin 3-O- |
| | | | | | glucosyltransferase 2 |
| NC_044377.1 | 76416328 | 76418041 | LOC115695871 | XP_030478823.1 | anthocyanidin 3-O- |
| | | | | | glucosyltransferase 2 |

### Example 6

### Identification of Additional SNPs in QTLs

Based on the GWA study findings from the mixed population of cannabis and the validation of those finding by GWA in two F2 populations segregating for the purple color trait, the inventors identified a QTL at 68717484 to 77040783 on chromosome NC_044377.1 with reference to the CS10 genome responsible for the regulation of purple color. The inventors reasoned that additional SNP markers present in the custom SNP marker panel described in Examples 1 to 4 in the region of the QTL, but not identified in the GWA, could be used to evaluate this QTL. The inventors selected SNPs from the custom marker panel between position 70000000-78000000 on chromosome NC_044377.1, with reference to the cs10 genome. The SNPs selected included those that were previously identified as associated with the purple color trait in Examples 1 to 4 (Table 7).

The inventors conceived to evaluate the markers through the use of a training population of 234 diverse cannabis genotypes that included high THC varieties, low THC/high CBD varieties, and assorted hemp plants. The training population was grown and harvested in an open field in 2022. The inventors determined the genotypes and phenotypes of these plants.

DNA was extracted from about 70 mg of leaf discs from all the plants evaluated using an adapted kit with “sbeadex” magnetic beads by LGC Genomics, which was automated on a KingFisher Flex with 96 Deep-Well Head by Thermo Fisher Scientific. The extracted DNA served as a template for the subsequent library preparation for sequencing. The library pools were prepared according to the manufacturer's instructions (AgriSeq™ HTS Library Kit—96 sample procedure from Thermo Fisher Scientific). Targeted sequencing of a custom SNP marker panel based on the Cannabis Sativa CS10 reference genome was carried out on the Ion Torrent system by Thermo Fisher Scientific. The primers for the SNPs identified are provided in Table 5 and Table 11. The library pool was loaded onto Ion 550 chips with Ion Chef and sequenced with Ion GeneStudio S5 Plus according to the manufacturer's instructions (Ion 550™ Kit from Thermo Fisher Scientific).

At time of harvest, the color of the flowers of these plants was evaluated on a scale from 1-9, with 1 being most the green and 9 being the most purple.

The inventors evaluated the allelic effect for each SNP associated with an average phenotype of the plants sharing a common allele by GWA analysis as in the previous examples (Table 7). The GWAS was performed using GAPIT version 3 (Wang and Zhang, 2021) with the Blink model. Complimentarily, the inventors evaluated the allelic effect where purple and green were evaluated as a binary trait: plants that had a score of 1-4 were deemed to be green and assigned a 0, those with a score of 6-9 were deemed to be purple and assigned a score of 1. The allele effect was similarly evaluated for each SNP and associated with an average phenotypic value by GWA performed using GAPIT version 3 (Wang and Zhang, 2021) with the Blink model, (Table 8). The sequence associated with SNPs not defined in the previous examples are given in Table 9, together with primer sequences that can be used for amplification to determine the allelic variant (Table 10).

TABLE 7
Allelic effect table of SNP markers where the phenotypic values
are in a range from 1-9, where 9 is the most purple. The positions
of the SNPs on chromosome NC_044377.1 are provided with reference
to the CS10 reference genome as described herein. The LOD score for
the Blink model is provided as LOD_BLINK. Mean_1, Mean_2 and Mean_3
denote the average phenotypic value associated with Allele_1, Allele_2
and Allele_3, respectively, based on the scoring from 1-9 for purple
color, with 9 being most purple. Count_1, Count_2, and Count_3 denote
the number of plants that contributed to the average phenotypic value
of Mean_1, Mean_2, and Mean_3, respectively.

| | | | | | | | | | | | LOD_{—} |
|---|---|---|---|---|---|---|---|---|---|---|---|
| SNP | Position | Alleles_1 | Alleles_2 | Alleles_3 | Mean_1 | Mean_2 | Mean_3 | Count_1 | Count_2 | Count_3 | BLINK |
| common_4479 | 72194335 | AA | AC | CC | 2.98276 | 3.42 | 3.22535 | 58 | 100 | 71 | 0.435151 |
| common_4481 | 72547365 | CC | CT | TT | 3.72656 | 2.54762 | 2.82353 | 128 | 84 | 17 | 1.2923627 |
| common_4483 | 72698293 | CC | CT | TT | 3.0354 | 3.39785 | 3.64706 | 113 | 93 | 17 | 0.4286138 |
| common_4484 | 72776852 | AA | AG | GG | 3.34043 | 3.33684 | 3.03371 | 47 | 95 | \|89 | 0.2538411 |
| GBScompat_{—} | 72805723 | AA | AC | CC | 3.32759 | 3.39535 | 1.91304 | 116 | 86 | 23 | 2.043402 |
| rare_164 | | | | | | | | | | | |
| common_4485 | 72948534 | AA | AG | GG | 2.85 | 3.29348 | 3.53247 | 60 | 92 | 77 | 0.3931966 |
| rare_551 | 73008201 | GG | GT | TT | 3 | 4.91667 | 6 | 202 | 12 | 6 | 0.2812617 |
| common_4487 | 73084793 | CC | CT | TT | 4.91667 | 3.36667 | 2.66372 | 24 | 90 | 113 | 0.4889833 |
| GBScompat_{—} | 73090007 | AA | AG | GG | 3.08911 | 4.5 | 4 | 202 | 22 | 2 | 0.8007105 |
| rare_165 | | | | | | | | | | | |
| common_4494 | 73826567 | AA | AC | CC | 3.28161 | 2.9697 | 3.16667 | 174 | 33 | 12 | 0.440787 |
| common_4495 | 73977781 | CC | CG | GG | 3.49524 | 3.24638 | 2.60417 | 105 | 69 | 48 | 0.706105 |
| GBScompat_{—} | 74049363 | AA | AC | CC | 3 | 2.96774 | 3.38776 | 17 | 62 | 147 | 0.3076736 |
| common_874 | | | | | | | | | | | |
| common_4499 | 74270488 | AA | AG | GG | 2.53846 | 2.57143 | 3.61184 | 13 | 63 | 152 | 0.5813504 |
| common_4500 | 74383125 | AA | AC | CC | 2.86207 | 3.33333 | 3.31429 | 29 | 93 | 105 | 0.6086946 |
| common_4502 | 74475496 | CC | CG | GG | 3.03061 | 3.43158 | 3.375 | 98 | 95 | 32 | 0.3154603 |
| common_4504 | 74637356 | CC | CT | TT | 4.48571 | 3.12903 | 2.88636 | 35 | 93 | 88 | 1.0491576 |
| common_4513 | 75673038 | AA | AC | CC | 2.78049 | 3.7037 | 6.81818 | 123 | 81 | 11 | 1.4418846 |
| common_4514 | 75675433 | AA | AG | GG | 4.24 | 3.06593 | 2.6 | 50 | 91 | 65 | 1.144972 |
| common_4515 | 75775693 | CC | CT | TT | 3.25333 | 3.75281 | 2.5 | 75 | 89 | 56 | 0.2767248 |
| GBScompat_{—} | 75779139 | AA | AG | GG | 1.46154 | 3.18868 | 3.46897 | 13 | 53 | 145 | 0.6210447 |
| common_876 | | | | | | | | | | | |
| common_4516 | 75858208 | CC | CT | TT | 4.02419 | 2.14103 | 2.22727 | 124 | 78 | 22 | 1.5233162 |
| common_4517 | 75907528 | AA | AC | CC | 2.73684 | 3.21053 | 3.81159 | 57 | 95 | 69 | 0.0354086 |
| common_4518 | 75977378 | CC | CT | TT | 3.75701 | 2.87778 | 2.41379 | 107 | 90 | 29 | 0.0197589 |
| common_4519 | 76201791 | AA | AG | GG | 3.64286 | 3.14706 | 2.3871 | 98 | 102 | 31 | 0.2129968 |
| GBScompat_{—} | 76400765 | AA | AG | GG | 2.84783 | 2.51852 | 3.97826 | 46 | 81 | 92 | 0.0507506 |
| common_877 | | | | | | | | | | | |
| common_4522 | 76434922 | CC | CT | TT | 3.5 | 3.65385 | 2.19672 | 40 | 104 | 61 | 0.4662512 |
| rare_556 | 76445504 | AA | AC | CC | 1 | 1.875 | 3.37619 | 2 | 16 | 210 | 1.7539793 |
| common_4525 | 76757670 | AA | AC | CC | 3.53846 | 3.50435 | 2.63014 | 39 | 115 | 73 | 1.4364765 |
| GBScompat_{—} | 76764686 | AA | AG | GG | 3.90217 | 3.0098 | 2.22857 | 92 | 102 | 35 | 0.5429001 |
| common_878 | | | | | | | | | | | |
| common_4526 | 76803155 | CC | CT | TT | 3.89796 | 3.63726 | 2.29268 | 49 | 102 | 82 | 2.0458712 |
| common_4527 | 76935329 | AA | AG | GG | 1.94 | 3.21831 | 5.02778 | 50 | 142 | 36 | 2.8485841 |
| rare_559 | 76935555 | CC | CT | TT | 3.28922 | 1.875 | 1 | 204 | 16 | 2 | 0.2341438 |
| common_4528 | 76959458 | AA | AG | GG | 2.94737 | 2.98333 | 3.67143 | 38 | 120 | 70 | 0.1685708 |
| GBScompat_{—} | 77040784 | AA | AT | TT | 3.2807 | 3.31325 | 3.18868 | 57 | 83 | 53 | 0.7451149 |
| common_879 | | | | | | | | | | | |
| common_4534 | 77431065 | AA | AG | GG | 3.86957 | 3.22018 | 2.34884 | 69 | 109 | 43 | 0.3647421 |
| common_4540 | 77925821 | CC | CG | GG | 2.80952 | 3.78295 | 2.425 | 21 | 129 | 80 | 0.2980371 |

TABLE 8
Allelic effect of SNP markers where the phenotypic values are binary, where 0 is green and 1 is purple. The positions
of the SNPs on chromosome NC_044377.1 are provided with reference to the CS10 reference genome as described herein. The LOD score for the Blink
model is provided as LOD_BLINK. Mean_1, Mean_2 and Mean_3 denote the average phenotypic value associated with Allele_1, Allele_2
and Allele_3, respectively, based on 0-1 scoring for purple color, with 0 being green and 1 being purple . Count_1, Count_2, and
Count_3 denote the number of plants that contributed to the average phenotypic value of Mean_1, Mean_2, and
Mean_3, respectively.

| | | | | | | | | | | | LOD_{—} |
|---|---|---|---|---|---|---|---|---|---|---|---|
| SNP | Position | Alleles_1 | Alleles_2 | Alleles_3 | Mean_1 | Mean_2 | Mean_3 | Count_1 | Count_2 | Count_3 | BLINK |
| common_4479 | 72194335 | AA | AC | CC | 0.1923077 | 0.278481 | 0.1896552 | 52 | 79 | 58 | 0.24254795 |
| common_4481 | 72547365 | CC | CT | TT | 0.3168317 | 0.1351351 | 0.0714286 | 101 | 74 | 14 | 0.50028911 |
| common_4483 | 72698293 | CC | CT | TT | 0.1666667 | 0.2763158 | 0.2941177 | 90 | 76 | 17 | 0.11522712 |
| common_4484 | 72776852 | AA | AG | GG | 0.2972973 | 0.1948052 | 0.2207792 | 37 | 77 | 77 | 0.9433052 |
| GBScompat_{—} | 72805723 | AA | AC | CC | 0.2325581 | 0.2763158 | 0 | 86 | 76 | 23 | 1.72423577 |
| rare_164 | | | | | | | | | | | |
| common_4485 | 72948534 | AA | AG | GG | 0.106383 | 0.2666667 | 0.2794118 | 47 | 75 | 68 | 1.44299889 |
| rare_551 | 73008201 | GG | GT | TT | 0.1952663 | 0.375 | 0.75 | 169 | 8 | 4 | 1.04201765 |
| common_4487 | 73084793 | CC | CT | TT | 0.5294118 | 0.2567568 | 0.1340206 | 17 | 74 | 97 | 0.59584442 |
| GBScompat_{—} | 73090007 | AA | AG | GG | 0.1987952 | 0.4444444 | 0.5 | 166 | 18 | 2 | 1.18315367 |
| rare_165 | | | | | | | | | | | |
| common_4494 | 73826567 | AA | AC | CC | 0.2312925 | 0.2692308 | 0 | 147 | 26 | 8 | 1.12330031 |
| common_4495 | 73977781 | CC | CG | GG | 0.2857143 | 0.25 | 0.0731707 | 84 | 56 | 41 | 0.36462765 |
| GBScompat_{—} | 74049363 | AA | AC | CC | 0.1428571 | 0.2 | 0.2520325 | 14 | 50 | 123 | 0.22400558 |
| common_874 | | | | | | | | | | | |
| common_4499 | 74270488 | AA | AG | GG | 0.1666667 | 0.1296296 | 0.2868853 | 12 | 54 | 122 | 0.00373675 |
| common_4500 | 74383125 | AA | AC | CC | 0.1363636 | 0.2467533 | 0.25 | 22 | 77 | 88 | 0.56459361 |
| common_4502 | 74475496 | CC | CG | GG | 0.2159091 | 0.2666667 | 0.2173913 | 88 | 75 | 23 | 1.18774325 |
| common_4504 | 74637356 | CC | CT | TT | 0.4782609 | 0.2207792 | 0.1666667 | 23 | 77 | 78 | 1.2776725 |
| common_4513 | 75673038 | AA | AC | CC | 0.1386139 | 0.3125 | 0.9 | 101 | 64 | 10 | 1.72169909 |
| common_4514 | 75675433 | AA | AG | GG | 0.4411765 | 0.1866667 | 0.1666667 | 34 | 75 | 60 | 0.217973 |
| common_4515 | 75775693 | CC | CT | TT | 0.2739726 | 0.3333333 | 0.0454546 | 73 | 63 | 44 | 1.25650183 |
| GBScompat_{—} | 75779139 | AA | AG | GG | 0 | 0.2083333 | 0.2678571 | 13 | 48 | 112 | 0.08287739 |
| common_876 | | | | | | | | | | | |
| common_4516 | 75858208 | CC | CT | TT | 0.3820225 | 0.0675676 | 0.0476191 | 89 | 74 | 21 | 0.85831711 |
| common_4517 | 75907528 | AA | AC | CC | 0.12 | 0.2588235 | 0.2978723 | 50 | 85 | 47 | 1.12405489 |
| common_4518 | 75977378 | CC | CT | TT | 0.2987013 | 0.2073171 | 0.0714286 | 77 | 82 | 28 | 0.33794508 |
| common_4519 | 76201791 | AA | AG | GG | 0.3 | 0.2247191 | 0.0967742 | 70 | 89 | 31 | 0.16517653 |
| GBScompat_{—} | 76400765 | AA | AG | GG | 0.0810811 | 0.1527778 | 0.3428571 | 37 | 72 | 70 | 0.02423832 |
| common_877 | | | | | | | | | | | |
| common_4522 | 76434922 | CC | CT | TT | 0.2941177 | 0.3209877 | 0.0535714 | 34 | 81 | 56 | 0.37643106 |
| rare_556 | 76445504 | AA | AC | CC | 0 | 0 | 0.2485549 | 2 | 13 | 173 | 1.30658963 |
| common_4525 | 76757670 | AA | AC | CC | 0.1851852 | 0.3232323 | 0.0819672 | 27 | 99 | 61 | 2.04236757 |
| GBScompat_{—} | 76764686 | AA | AG | GG | 0.36 | 0.1975309 | 0.030303 | 75 | 81 | 33 | 0.2104317 |
| common_878 | | | | | | | | | | | |
| common_4526 | 76803155 | CC | CT | TT | 0.3846154 | 0.2894737 | 0.0779221 | 39 | 76 | 77 | 2.06280081 |
| common_4527 | 76935329 | AA | AG | GG | 0.0212766 | 0.25 | 0.5 | 47 | 116 | 26 | 0.91816389 |
| rare_559 | 76935555 | CC | CT | TT | 0.2321429 | 0 | 0 | 168 | 13 | 2 | 0.09378689 |
| common_4528 | 76959458 | AA | AG | GG | 0.2857143 | 0.1666667 | 0.2941177 | 35 | 102 | 51 | 0.99598663 |
| GBScompat_{—} | 77040784 | AA | AT | TT | 0.2093023 | 0.2318841 | 0.2790698 | 43 | 69 | 43 | 2.08497285 |
| common_879 | | | | | | | | | | | |
| common_4534 | 77431065 | AA | AG | GG | 0.3728814 | 0.2022472 | 0.0571429 | 59 | 89 | 35 | 0.58252157 |
| common_4540 | 77925821 | CC | CG | GG | 0.1578947 | 0.3173077 | 0.1044776 | 19 | 104 | 67 | 0.37734319 |

The individual SNPs that the inventors identified for this region in Examples 1 to 4 above and the additional SNPs in this region as set out in this Example 6 have predictive power alone in determining whether plants will display a purple or green phenotype based on the genotype of the SNP, as demonstrated in the validation of the markers in the training population where the phenotypic values for purple are given on a scale from 1-9 or as a binary trait, with green=0 and purple=1. When taken as a binary trait as shown in Table 8, the SNPs “common_4519”, “common_4525”, and “common_4500” have an ˜90%, 92%, 87% accuracy in selecting for green plants, respectively. The predictive power of these markers alone for selecting for plants with a propensity for being purple is highly informative. Additionally, the SNP markers “common_4513”, “rare_551”, and “common_4487” are highly predictive for selecting plants that will be purple at the time of flowering. Furthermore, markers that have a high allelic effect are particularly useful in selecting for purple or green cannabis flowers and plants. The inventors identified the following SNP markers as having a large allelic effect: “common_4487”, “common_4504”, “common_4513”, “common_4516”, “rare_556”, “GBScompat_common_878”.

The inventors tested the prediction of the phenotype using genotype information with individual markers in the region of the QTL for purple color on chromosome NC_044377.1. The inventors used the genotype and phenotype data from the training population treating color as a binary trait for each SNP marker identified in Table 8 to calculate a false positive rate, i.e., the percentage of plants that do not fit the predicted phenotype for a given allele variant “X”, and the false negative rate, the percent of plants that are the alternative allele or heterozygous but that display the phenotype predicted for “X” (Table 9). By way of example, for SNP marker “common_4513”, the genotype CC predicts purple color (Table 3). “Common_4513” has a false positive rate of 0.56, meaning that 0.56% of the plants that were predicted to be purple by this homozygous allele are green. “Common_4513” has a false negative rate of 16.38, meaning that 16.36 percent of the plants that are purple do not have the alleles CC (Table 9). A low false positive rate can be exploited by breeders conducting marker assisted selection to vastly reduce the number of plants in a breeding population and increase the likelihood that the plants under selection contain the trait of interest. A low false negative rate can facilitate marker assisted selection by improving the likelihood that the allele selected for is tightly linked to the trait of interest. However, a high false negative rate can be tolerated, as the plants with the allele state not selected for will not be selected. The inventors identify herein three additional markers meeting the criteria of a p value under 0.05 and with a false positive rate lower than ˜5% that can be used individually for marker assisted selection of the purple trait: “common_4513”, “common_4487”, and “GBScompat_rare_165” (originally identified in Example 3 and Example 4). However, all of the SNP markers in Table 7 and Table 8 can be used in marker assisted selection using the false positive rate and false negative rate provided in Table 9, as a guide in decision making.

TABLE 9
The false positive (false Pos Rate) and false negative rate (false Neg
Rate) for each of the SNP markers in Table 7 and Table 8, calculated
treating color as a binary trait. A p-Value is given for each SNP.

| | | false Pos | false Neg |
|---|---|---|---|
| SNP Marker | p-Value | Rate | Rate |
| common_4516 | 0.000000125 | 49.15 | 16.95 |
| common_4513 | 0.00000757 | 0.56 | 16.38 |
| common_4527 | 0.0000489 | 52.54 | 0.56 |
| common_4522 | 0.0000559 | 29.94 | 17.51 |
| common_4526 | 0.0000623 | 40.68 | 2.82 |
| common_4515 | 0.000159526 | 50.85 | 0.56 |
| common_4481 | 0.000347863 | 37.85 | 3.95 |
| GBScompat_common_877 | 0.001101148 | 51.98 | 11.86 |
| common_4525 | 0.001284114 | 47.46 | 2.26 |
| common_4514 | 0.001461061 | 59.32 | 7.91 |
| GBScompat_common_878 | 0.002130623 | 59.89 | 0.56 |
| common_4534 | 0.002333597 | 20.34 | 9.04 |
| common_4487 | 0.003226859 | 3.95 | 15.82 |
| GBScompat_rare_165 | 0.004075931 | 5.08 | 15.82 |
| GBScompat_rare_164 | 0.004698482 | 12.99 | 20.34 |
| common_4485 | 0.00549864 | 23.73 | 19.77 |
| common_4540 | 0.006426547 | 46.89 | 3.39 |
| common_4495 | 0.010119266 | 19.21 | 18.64 |
| common_4504 | 0.034254239 | 67.8 | 4.52 |
| common_4483 | 0.038281704 | 35.03 | 6.78 |
| rare_556 | 0.042274055 | 7.91 | 21.47 |
| rare_559 | 0.043724158 | 8.47 | 19.21 |
| GBScompat_common_876 | 0.072674338 | 7.34 | 19.77 |
| common_4518 | 0.07379385 | 14.69 | 19.77 |
| common_4499 | 0.085904153 | 31.07 | 16.38 |
| common_4519 | 0.09383373 | 15.82 | 19.77 |
| common_4517 | 0.097221726 | 23.73 | 17.51 |
| rare_551 | 0.103409833 | 0.56 | 18.08 |
| common_4528 | 0.162412805 | 64.97 | 5.65 |
| common_4494 | 0.204738513 | 4.52 | 20.34 |
| common_4484 | 0.35627679 | 65.54 | 5.08 |
| GBScompat_common_879 | 0.377179454 | 46.33 | 6.21 |
| common_4500 | 0.41538422 | 10.73 | 19.77 |
| common_4502 | 0.467222402 | 37.85 | 12.43 |
| common_4479 | 0.53421441 | 56.5 | 4.52 |
| GBScompat_common_874 | 0.702463655 | 28.25 | 14.12 |

The reference or context sequence for each of the additional SNPs identified in Table 7 and Table 8 is provided in Table 10 with reference to the CS10 genome. In Table 11, PCR primers designed to amplify each of the regions containing these SNPs, with reference to the CS10 genome, are provided in order for the allelic variant to be determined.

TABLE 10
Detailed information of each of the additional SNPs associated with purple color in
Cannabis as provided in Table 7 and Table 8. The “context sequence” is provided with
the SNP given in brackets. All of the sequences and alleles are provided with
reference to the plus strand.

| SNP | Reference Sequence |
|---|---|
| common_4479 | TGCTTCTCAGAGGGAGTGTGTGTGAGAGAGAAGTGGCCGAAGCCCGTGGAGTGTGTTGTT |
| | GAGGTGGGCCGTACCGATGGAGACGAGTGCCCAAACGACGTCGTTCGGAGAACAACGCG |
| | GTGCCGGAGGTAAGCTCAAGAGGCCGGCTCGCAAGCCACCGTCAACTCCCTACTCTCGTC |
| | CTCCGGCGCTACCGACCCAAAT[A/C]GATAGGGGTCAGCGGCGATGGTTGTCCAAGCTCGT |
| | TGATCCCGCATACCGCCTCATTAGTGATGGCGCCTCCATCTTCTTTCCGAATTTCTTCTCAA |
| | AATCACCTTTCACCATTGATGCCAACACTGAAGATCACGGTGGGTTTTACATATATATTCAAC |
| | TCTCTATGCTTCTTAGTCAAGCTTTCTTTCGATTGATTAGG |
| | (SEQ ID NO: 142) |
| common_4481 | AGCTATACTGTAATATCCCTCATTGATTTGTATAATAAACTTGAATCAGAGAATTGATGAAGG |
| | TTGTACCAAATTTTAGAAAATCTCTGTTTGATAATTTTCCCATGGTTATATGGTGTAGTGTAGA |
| | AAAAGTGTTATTGAATACTATGTTGTTTTTGGTGTTTTGTGGTTGAAGTTTCTGTTTGTGTTTG |
| | GTTTTCCAGT[T/C]TCTCACAGAGCACAAGCATTTACTGGAACGTATGGAATAAATTACGGGA |
| | GAATTGCAGATAACATCCCTTCTCCGGATGAAGTTGCTACTCTTCTCAGAGCAGCTAAGATA |
| | AAGAATGTTAGAATATACGATGCAGATCACAGTGTTCTCAAGGCCTTTAGTGGGACTGGACT |
| | TGAATTAGTAATTGGACTTCCAAATGGA |
| | (SEQ ID NO: 143) |
| common_4484 | TCTCGTGAAAACACTTCCACTCATCTTTCGTCCACCCATTCTTGCATGCCGCTTATTTGGTAT |
| | TTGTGGCAGAGGGTACATTGACATATTTGTTGTGCAACATGATGACTGCCATCCACCGCTCC |
| | CCCATTTGTAACACTGTCGAGAAACACCAGTGCAGGAGCAAACCGGCACTGGCATGATAGA |
| | CTCATCAAAAGTTAT[A/G]AGGTTCAAGCCCACATCCTGACTATCCCATTCTGTTCTATACTTT |
| | GTGCCATCAGAGGTAGCTCGTCTGTTCAGATCCTCTGCTACTTTCTTTCGCGGTGGCTTTGA |
| | CTCCCTGGATGAATGTCCCTTGTTCACCTTTGGTCGCTTTGTCTGAGGTGACTTGATAGCAT |
| | CAGAAGATATAACTGTAATGGGGAAGGCATCG |
| | (SEQ ID NO: 144) |
| common_4485 | CGGCTACGCTTTCGCCGGGGATAGCTCTCTTCCCGCGCCGGTCATATTTTCCGGCGTTCCT |
| | CCGGCGACAACCGCTACTGCCACCGCTTGGTCACCTTCCTTATCGTCTGCTCTCTACAAAG |
| | TCGATGGGTGGGGCGCACCTTACTTCGCCGTCAACTCTTCCGGCAACGTCGCCGTTCGCC |
| | CTCACGGCGCTGGAACTTT[G/A]GCGCACCAGGAGATTGATTTGCTGAAAATTGTAAAGAAG |
| | GTTTCGGATCCGAAATCTAAGGGCGGGTTAGGTTTGCCCCTTCCGCTCGTTATTCGGCTTC |
| | CTGATGTGCTTAAGAACCGCCTTGAGTCTCTCCAGGCGGCGTTCGATTTCGCAATCCAGTC |
| | GCAGGACTATGAAAACCATTACCAGGGTGTTTACCCTGTG |
| | (SEQ ID NO: 145) |
| rare_551 | CGTGAAGCTGTTTCCCTTGATGACTCAGTCCCAATCGAGCACACGACTTGAGAACAAACGG |
| | GTACGTGAACTTATCGGGTCGGGTCGGGCTGTTACGAGCCATATAGAGGAAAAAGTGTAGA |
| | GCTTTGATTGGGTCGGGGCTCCGAGAGTAGGCACGGATCATGGTGTTGTAGTGGAATGAAT |
| | TGGGGGATGGGATAGAGT[G/T]GAGAATGAGACGGGCATAAGTGAGGTCGCCATTGGAGGA |
| | AAGAGCTGAGAAGGTGAAGATGTTGGTAAGGCTCTTTTGTTGGGTTTTGCCTGATTTGAGTG |
| | TTTGGGCATGGAGTTGCAGAGCTTCGGGCATGGAGTTTATCTCCATCTTCGGAGGTGGTGG |
| | CGGCGCCGCCAATGTGCATGGCAGAGCTCCGATGAGAGT |
| | (SEQ ID NO: 146) |
| common_4494 | TATAATTATGCTTATAATACCATTTTTATCACCTTTCTCCAATCTTTAGTTCTCTGATTAATTTA |
| | CTTCCTATAAACAGAGTAACTTCGAGAGAGAGGGAGAGATATATATCTTTTTTGTGCCACCG |
| | CTTCACTCACATACCATTCCATGTCGTGAAGTGCATATAGGATTAGGTCCATCCACGTAGCT |
| | AGCGAAATATCC[A/C]TGATATTTTTAGCACAAAAACAGATATTGTCCAAATTTCTCTATAAAA |
| | AGCAACGAATCTTATATTCTTCCTCTACTTGCCCAAAATAGTAATAATGGATAGAGCTTTAAC |
| | TGGTTTGATCTGCTGCTTCGTCCTTACGGTGGTCATCTTTACAGAGACTGCAGCTGCAGCAA |
| | TACATACCGTAGGAGACCACATAGGCTG |
| | (SEQ ID NO: 147) |
| common_4495 | TGAAAAAGATTGTGGACAATATTATAACTCAAAAACGTATAATTTAAAATAATTAATACATATA |
| | GTATACTACTTTTTCTCTTTTCACAACACGTGTATCATTTTGTGGTGCTCTCTTCTCTTGTGTC |
| | TAATTATTATATAAAGGATAGAACAAGTTCCAAAACATAATAAAACCCACCGAGTGCTAATTA |
| | ATAAAGTTCT[C/G]AAGCTCTCTTCTTCTTCTTGCTTCTCCTCTACTTGGCCAAAATAATAGTT |
| | TGAGAGAAAAAGAGGTGGGAAGGGACGACCTTTGAACACAGTTGATTTTCATAATGGCAAG |
| | AGGTTTAGTGGGTTTGATCTGCTGTTTCGCCCTTACGGTGGTCATCTTTACAGATAGTACAG |
| | CGGCGGTGACACATACGGTTGGAGACAC |
| | (SEQ ID NO: 148) |
| GBScompat | CGCTTTTAGTATTTATATTGTACCAACTCAATTATCATGAACATTTGTTTCATCTTTACCCGTT |
| common_874 | ATGTACTTGTAGGGTATTGCAAAGATGGGCTGACCATGTCCTTCATATAGAACACAACTTGT |
| | GGAGGTATCACTATTTTTCAATTTTGTTTTCACGCTAGATTCTTCTTTCAACGTGTTCTGAGC |
| | CGATCAAGGTCC[C/A]ATGCAGGGCTCTGATTTTGTATGTTGCGGCTGGAGCACTGCCAGAG |
| | GCATTGGCAGCTCTTCGTGAAGCACAACAACCAGACACAGCTGCCATGTTTGTCCTTGCTT |
| | GCCGTGAAATACACTCAGAAATCATCTCCAGTTTGGACTTGGATGATGAAACTAGTTCCTCT |
| | ACCAATGAGAAACTGCTTAAGTTGCCTGGTTT |
| | (SEQ ID NO: 149) |
| common_4499 | CGAAAAGCCTATCTGAAATCTTAGTTCTACAGATAGGACACTCTGAACAAGCTAATGAACAA |
| | GATTTACATACTGCAAAACAAAATGATAAATATGATTAGTTTTAATTTAATGTTAAAAGTGAAA |
| | TCAAGCATGTAGGATTCATAGGAATGATGATGACTGCTTACAACAAAAATGGCGACACGGCA |
| | AAAGAATTGCAGC[G/A]GTCGGGGATTCAAAACATACTTTACACATGTGAGAATTGGCATCTC |
| | CATTCCCCATTTGTTTCAGCTCCTTTTCCTTCATCTCTTGCATTCGAGCCTGAAAACAACATA |
| | CATCTTGAGTTTGAGATACCTTAACACTTTTTCGAGAACTTTTTAGTTTCTTGATCAACAAATG |
| | CATTTTCCTTGTGGATTTAAAATTATGC |
| | (SEQ ID NO: 150) |
| common_4502 | GATAATTTGATCTTACCTTGTTGTGTACCATAAGTAATCAGTGGAGTCTCTGGTGACATTTAA |
| | CTGCTCTAAGAGACCAACTGCTGTGATTTTTGTATCTTCATCCACTGAAGACATGTCTTCACT |
| | AAATGTCTCCCATGAGTACAATGCACCTTTCAAGGGCGACATTTGCGTTCGAGTCATTTGTA |
| | CTCTCACCTGTTA[G/C]GAAGTGACAGATGCCCGTTATAGACAATGAGTGAAGTAGCCAATG |
| | CATCTTACATATAGTACACGATCAACCCTGTGATAAAGCGGGCTAGCCCTGATATGGTATTA |
| | TGTTCTTATATCAAGCTCAATAATTAATCGTTTTCTTATGGCCCATTTAGTCTCAGGGTCAGT |
| | CCTGTTATAGTATTATGTTCTTACACATTAT |
| | (SEQ ID NO: 151) |
| common_4514 | CACTTTAAGTTATAAATTACGTTGTAACTAAAAGTAAAAATCTTTGTAGTGTAAATTTATATAT |
| | ATTTACCTCGGAGACCATGTCATTGAAAACTCTTCCCATTATTTCCATCTTATGTTTAGGATC |
| | ATTACTCATAACACTTCCCATCATGGTTAGTACCATACTCCCACCACTTACTATTTCCTCTGA |
| | ACGACATCTTA[A/G]AAAGCTTGTGAAATCCTCTTGAAATTGATTCAAGTAAGCTTTCACAACT |
| | GAATTAGGGCTTCTTTTTGTAATATAAATGTTGCCTTTGTTAAGTGCTTCTCCTGTTTCCCCA |
| | ACCAAACCACTTGGAACCTTCAAAACAACATTAATTCACATAATTAATAAACTAAAACCTTATA |
| | AAAAGAAAGGGTAAATTTCAATTTT |
| | (SEQ ID NO: 152) |
| common_4515 | TTAACGACCAGAAAAGTTCAGGGGGCATGATTTAATACATGTCAAAGTTCGGAGAGTAAAAA |
| | TCCTAATTAGCCTAAAAAAAACTAAGCAAGACAGTACTGATATATATTTATATGTTTATATAGG |
| | TGCTCATACATTTGGAAGGGGTCAATGTGGAACCTTCAGTGGAAGATTATTCAATTTTAGTG |
| | GCACAGGTGCTCC[T/C]GATCCAACCATAGAGGAAAACTACTTGAGATTATTACAAGATCTAT |
| | GTCCTCAAGGAGGAAACGCCTCTATCTTAACAGATTTAGACCCCACCACACCAGATTTGTTT |
| | GACAGAAACTACTACTCAAATCTTCAACAACACATGGGATTGTTCACAAGTGACCAAGAGCT |
| | GTTTTCAAGCCCATTAGCAGCAAATGACACT |
| | (SEQ ID NO: 153) |
| GBScompat_ | AACATAAAAGAGATTGCAGAAAACTTTTCCAAGGTACTCTTATAAACAGTCATAATTAATCAC |
| common_876 | AGATTTGTTAGAACAATTATGCTCACTTCCTAAAACTCAACGATGAATTTGGCTGTACATTGT |
| | AATACAGACTGATTGATTTTATCTTTCTCTATCGACGCAGCTTGCTTGCAATGCACATACAAT |
| | TTGTGATAATGA[G/A]TTGAGACCCCTGGGGACCGGGCTGTTTCCTGTCATATCTATTATTAA |
| | CCACAGGTAACTAGATCAGTTCAATAATGCTGTTTAACAAATTTTAGTTCATTGATATGTTTTA |
| | GGTACCATTCTCCTGATTTCAGTCTGTTGAATGTTTAAGCTGCTTGCCAAATTCTGTTCTGGT |
| | TTTTGAGGGAAAGTTAGCTGTCGTGCG |
| | (SEQ ID NO: 154) |
| common_4516 | TTTGGTGCGAAAGTTTTGAATTTACAGTTTTGTCCCTTTTCTGTTCTTATTTTATTTTTACAATC |
| | TGAAACTGAAGAAGAAGAAGAAGAAGATAGATACTGAGTTCGGACCGAGTCAATTATGGATT |
| | CTAACTCAGATACAACGAGTCCTAAAACACCGCTTTTGGGACCAGAAGCTCGGAGACCGAG |
| | TGATAACGGTCGC[C/T]GGAATCGCCGACTCAGTCGAAGATACTCTGTTAACTCGCTCAGGA |
| | CCGAATTCGTTTCCAGGCTCCCGGAAAAAGTCCGTTCTGGTTTCGACGTTGAGTCCAGTCC |
| | TTATGATATCGACCTAACTCGGACCACTGGCTTGAGCGGAGGTTTGTTTGAAATTTCAATTC |
| | AAGAAAAAAAACAATACTCTGTTTGGTTACTGA |
| | (SEQ ID NO: 155) |
| GBScompat_ | AGAGGAAGCTAGTGACGAAGATGCACACTTGACATAAACCGAAATCAACGAATTCAAAACC |
| common_877 | GAAGTAACAAAACACAATCCGAACTTCACAACCGCGCAATGCATTTGTTGACACTGCCTTTC |
| | ATCATCAACTACAAGTCCAAAAGCACTAAGAACACTAGATAAAGTAAAGTCATCAACCCCAA |
| | CACTAACCCTCCTCAT[G/A]TCATGAAATGTTTGAATAGCAGCAAAGCCATCATTGTTATGAG |
| | AACAACAAGTGATCATGGCATTGTAGAAAACAGTGTCTCTCATGGTCAATGGAGTAGCAAAG |
| | AACACTTCCCTTGCCAACTTAAGATTCCCAGCTACAGAGTAAGCTGCAATCATAGTCGTTCT |
| | AGAAATGATATCTGGTTTAGGAATTTGGTCGAAC |
| | (SEQ ID NO: 156) |
| rare_556 | TGGGAAATACGGCTCTTCATTTGGCTGCTTTGATTGGGAACATTCCTATGTGTCGGGCCATC |
| | ACCAAACCCTATTCTGATCAGGCAGCTTCGTTGATTGAAATTGAGAATAATATGGGTGAAAC |
| | TCCTCTCTTCTTGGCTTCTCTTCATGGCAAGAAGGACACTTTTCTTTATCTTAACTCAATCTT |
| | GAAGAAAACTCGGC[C/A]TGAAACCGCGATTACTTGTTGTAGAAGGCATAATGGTGATACTG |
| | TTCTACACTGTGCTATAGATGGCGAGTTCTTCGGTAAGTCTAAGTAGAATTTATTACTTCTTT |
| | TAAATTAATTCTCATTGAACATGCTCTTAATTGTAAATTGAATTTTGAATTGAAATTAAACTCA |
| | AAAATTAATTGTCAATTAAAATTTTGTTAC |
| | (SEQ ID NO: 157) |
| GBScompat_ | AAACAGTTCGTAAATGTATGTATTTATAGGCAAGGTCGGGTGGCTCGGTGGACCTGAATTGT |
| common_878 | TAAGAACACGTCGGATCAAGTTATTATTGAATGTCTCAGCATTTTCAAGATTGGCATCCGGTT |
| | CTTTGGCACCACCGAGCCATGGCCAACCAGCTTCGGTAACAACAACAGGAATCCCCGAAAA |
| | ATTTGCAGCCTCCAT[G/A]GCATAATAGGTAGCATCCACCATTGCATCAAACATGCTACTATA |
| | GTGGAAAAGAGTGTTGGGATCAACAATCTGCTTAACCGAGGGAAGTGGTCGAAAAATAGCA |
| | TAATCAATCGGAAAAATGCCATCCCCTTCCGTGTATCCATAATATGGATACGCATTTAACATG |
| | TAATAGGAATTTGTGTTTTTCAAAAACTGAAGG |
| | (SEQ ID NO: 158) |
| common_4527 | AGAGGACTAAAATACTTGCATCAAGATTGTAGTGCGGCGATTTTACATTTTGACATAAAACCT |
| | CATAACATTCTTCTAGATTTTGATTTTTCTCCTAAAATTTCTGACTTCGGCCTTGCCAAGTTGT |
| | GGCAAAGGGATGGGAGTGGTGTATCACTGTTGAAGGGTAGAGGCACAATTGGATATACAGC |
| | ACCGGAGATGCAT[A/G]ACAGAAATTTTGGTGAAGTATCTTATAAATCTAATGTTTATAGTTAT |
| | GGAATGATGGTTTTAGAAATGGTGGGCGGAAGAAAAAATATTGATACTAGTGTTTCTCGAAC |
| | AAGTGCAATATATTATCCACATTGGGCACATAAGCATATCAATGATGATGATGATGGTGAGC |
| | TCTTGAAAAATATTTGTGAAGAAATAATGG |
| | (SEQ ID NO: 159) |
| rare_559 | ATAAATCTAATGTTTATAGTTATGGAATGATGGTTTTAGAAATGGTGGGCGGAAGAAAAAATA |
| | TTGATACTAGTGTTTCTCGAACAAGTGCAATATATTATCCACATTGGGCACATAAGCATATCA |
| | ATGATGATGATGATGGTGAGCTCTTGAAAAATATTTGTGAAGAAATAATGGAAAATGAAGAT |
| | GATGAAATGTTGG[C/T]GAGAAAGATGATTATTGTAAGCTTATGGTGCATTCAAGCTATGCCA |
| | TCCAATCGTCCCTCCATGTTTAATGTCGTACAAATGATGGAAGGGAGTTTAGAATCTTTACAT |
| | ATGCCACCAAAACCAACATTGTCTTTACAAAAAGAGTAATATTATTTGTCATCTATATATATAT |
| | ATATTTGTATTTTCATACAACTTCTAGT |
| | (SEQ ID NO: 160) |
| common_4534 | TTGAGAATACTGAAAGCGCAATCAGAATACCGGTGGGGAATCGATAAGAACCTGCCGGTTT |
| | GTTCTTCCACATCCACTCCTGAGCATGCTGCTGATGCCCCTGAAATCACACACATTAAGAAT |
| | TAATTTGTGGTATCGAATTCTAAACACTAAAAAAACCGAAAAAGGAATGAAAGATTTGGGGG |
| | CTCTTTTTACCTGCAT[G/A]TTTTGATGACCCATCATTCCCGAAGCCTGATACTGGAATATTAC |
| | GTTTAATGGTCAGTAATCGGTAATAATGACAATTCTATTAAATCTCTTATGCTACTTAAAAAAC |
| | AAAAGAGAAAAAGAACTGACAGTCGAAGATAAAGAAGGCCGTGGCAGCTGCTGTTGAGGCT |
| | GCATTTGGAACTGTGTTTGATGCTGTATCTGC |
| | (SEQ ID NO: 161) |
| common_4540 | ATGACTACGATAGTATTTTATACCTGCAGCTGCAATACACATGGTTTGACCATCATCATCATC |
| | CCCAATGGCATATAGGAAATACCCACCAAGATGTTTCTTAGCATACTCGACCTTTTCATTTAT |
| | AGATTCTGAAGCTTCGTAGCCATACCAAGTATTTTTATCAGCCTTGTACATGCACTTGGTAAC |
| | ATCCTTATCATA[G/C]ATCCCACCTTCTGGATCAGGAATATCTTTATACGCGATGGGACCCGA |
| | ATAGGCAATAGTTGGTGCCCCAATGCTGGAATCATTTTCATCAGCCAATGTCCATTTATTTCC |
| | GTACAAGGGAATGCCCATAACTAGTTTACAGGTCGAAAATTCATTTGTAGTGAGCCAACTAT |
| | TAATAGAGTCTTCACTGCTGTTATTAGGG |
| | (SEQ ID NO: 162) |

TABLE 11
Targeted sequencing primers (5′ to 3′) for the additional SNPs identified in Table 7 and
Table 8, as described in this Example 6.

| SNP | Forward Primer 1 | Reverse Primer 1 | Forward Primer 2 | Reverse Primer 2 |
|---|---|---|---|---|
| common_4479 | CCGTGGAGTGTGT | CCCACCGTGATCT | CGTGGAGTGTGTT | CCCACCGTGATCT |
| | TGTTGAG | TCAGTGT | GTTGAGG | TCAGTGT |
| | (SEQ ID NO: 163) | (SEQ ID NO: 164) | (SEQ ID NO: 165) | (SEQ ID NO: 164) |
| common_4481 | CCCATGGTTATATG | GTCCAGTCCCACT | TCCCATGGTTATAT | TCCAGTCCCACTAA |
| | GTGTAGTGT | AAAGGCC | GGTGTAGTGT | AGGCCT |
| | (SEQ ID NO: 166) | (SEQ ID NO: 167) | (SEQ ID NO: 168) | (SEQ ID NO: 169) |
| common_4484 | TTCGTCCACCCATT | CATCCAGGGAGTC | TTCGTCCACCCATT | ATCCAGGGAGTCA |
| | CTTGCA | AAAGCCA | CTTGCA | AAGCCAC |
| | (SEQ ID NO: 170) | (SEQ ID NO: 171) | (SEQ ID NO: 170) | (SEQ ID NO: 172) |
| common_4485 | CGGTCATATTTTCC | GGAGAGACTCAAG | TATTTTCCGGCGTT | GGAGAGACTCAAG |
| | GGCGTTC | GCGGTTC | CCTCCG | GCGGTTC |
| | (SEQ ID NO: 173) | (SEQ ID NO: 174) | (SEQ ID NO: 175) | (SEQ ID NO: 174) |
| rare_551 | TCAGTCCCAATCG | CTGCAACTCCATG | TCAGTCCCAATCG | TCTGCAACTCCATG |
| | AGCACAC | CCCAAAC | AGCACAC | CCCAAA |
| | (SEQ ID NO: 176) | (SEQ ID NO: 177) | (SEQ ID NO: 176) | (SEQ ID NO: 178) |
| common_4494 | TTTTTGTGCCACCG | CAGCCTATGTGGT | TTTTTGTGCCACCG | GCCTATGTGGTCT |
| | CTTCAC | CTCCTACG | CTTCAC | CCTACGG |
| | (SEQ ID NO: 179) | (SEQ ID NO: 180) | (SEQ ID NO: 179) | (SEQ ID NO: 181) |
| common_4495 | TGTGGTGCTCTCTT | TCCAACCGTATGT | TGGTGCTCTCTTCT | TCCAACCGTATGT |
| | CTCTTGT | GTCACCG | CTTGTGTC | GTCACCG |
| | (SEQ ID NO: 182) | (SEQ ID NO: 183) | (SEQ ID NO: 184) | (SEQ ID NO: 183) |
| GBScompat_ | GGGCTGACCATGT | ATTTCACGGCAAG | GGGCTGACCATGT | AGCAGTTTCTCATT |
| common_874 | CCTTCAT | CAAGGAC | CCTTCAT | GGTAGAGGA |
| | (SEQ ID NO: 185) | (SEQ ID NO: 186) | (SEQ ID NO: 185) | (SEQ ID NO: 187) |
| common_4499 | CAGATAGGACACT | TCAGGCTCGAATG | CAGATAGGACACT | TCAGGCTCGAATG |
| | CTGAACAAGCT | CAAGAGA | CTGAACAAGC | CAAGAGA |
| | (SEQ ID NO: 188) | (SEQ ID NO: 189) | (SEQ ID NO: 190) | (SEQ ID NO: 189) |
| common_4502 | CAGTGGAGTCTCT | GGCTAGCCCGCTT | TCAGTGGAGTCTC | TAGCCCGCTTTATC |
| | GGTGACA | TATCACA | TGGTGACA | ACAGGG |
| | (SEQ ID NO: 191) | (SEQ ID NO: 192) | (SEQ ID NO: 193) | (SEQ ID NO: 194) |
| common_4514 | ACCTCGGAGACCA | TGGGGAAACAGGA | ACCTCGGAGACCA | GGTTTGGTTGGGG |
| | TGTCATTG | GAAGCAC | TGTCATTG | AAACAGG |
| | (SEQ ID NO: 195) | (SEQ ID NO: 196) | (SEQ ID NO: 195) | (SEQ ID NO: 197) |
| common_4515 | CCAGAAAAGTTCA | CAAATCTGGTGTG | CCAGAAAAGTTCA | AAATCTGGTGTGG |
| | GGGGGCA | GTGGGGT | GGGGGCA | TGGGGTC |
| | (SEQ ID NO: 198) | (SEQ ID NO: 199) | (SEQ ID NO: 198) | (SEQ ID NO: 200) |
| GBScompat_ | CTCTATCGACGCA | CGCACGACAGCTA | TCTCTATCGACGCA | CGCACGACAGCTA |
| common_876 | GCTTGCT | ACTTTCC | GCTTGC | ACTTTCC |
| | (SEQ ID NO: 201) | (SEQ ID NO: 202) | (SEQ ID NO: 203) | (SEQ ID NO: 202) |
| common_4516 | CTGAGTTCGGACC | AAACAAACCTCCG | ACTGAGTTCGGAC | CAAGCCAGTGGTC |
| | GAGTCAA | CTCAAGC | CGAGTCA | CGAGTTA |
| | (SEQ ID NO: 204) | (SEQ ID NO: 205) | (SEQ ID NO: 206) | (SEQ ID NO: 207) |
| GBScompat_ | GAACTTCACAACC | AGCTGGGAATCTT | GAACTTCACAACC | AGTTGGCAAGGGA |
| common_877 | GCGCAAT | AAGTTGGCA | GCGCAAT | AGTGTTCT |
| | (SEQ ID NO: 208) | (SEQ ID NO: 209) | (SEQ ID NO: 208) | (SEQ ID NO: 210) |
| rare_556 | GGCTCTTCATTTGG | ACTTACCGAAGAA | GGCTCTTCATTTGG | GACTTACCGAAGA |
| | CTGCTT | CTCGCCA | CTGCTT | ACTCGCCA |
| | (SEQ ID NO: 211) | (SEQ ID NO: 212) | (SEQ ID NO: 211) | (SEQ ID NO: 213) |
| GBScompat_ | GCTCGGTGGACCT | CACGGAAGGGGAT | GCTCGGTGGACCT | ACGGAAGGGGATG |
| common_878 | GAATTGT | GGCATTT | GAATTGT | GCATTTT |
| | (SEQ ID NO: 214) | (SEQ ID NO: 215) | (SEQ ID NO: 214) | (SEQ ID NO: 216) |
| common_4527 | TCAAGATTGTAGTG | TTCTTCCGCCCAC | CATCAAGATTGTAG | TTCTTCCGCCCAC |
| | CGGCGA | CATTTCT | TGCGGCG | CATTTCT |
| | (SEQ ID NO: 217) | (SEQ ID NO: 218) | (SEQ ID NO: 219) | (SEQ ID NO: 218) |
| rare_559 | AGAAATGGTGGGC | ACAATGTTGGTTTT | AGAAATGGTGGGC | AGACAATGTTGGTT |
| | GGAAGAA | GGTGGCA | GGAAGAA | TTGGTGGC |
| | (SEQ ID NO: 220) | (SEQ ID NO: 221) | (SEQ ID NO: 220) | (SEQ ID NO: 222) |
| common_4534 | TGCTGATGCCCCT | ACACAGTTCCAAAT | TGCTGATGCCCCT | CAAATGCAGCCTC |
| | GAAATCA | GCAGCC | GAAATCA | AACAGCA |
| | (SEQ ID NO: 223) | (SEQ ID NO: 224) | (SEQ ID NO: 223) | (SEQ ID NO: 225) |
| common_4540 | GCAGCTGCAATAC | ATGATTCCAGCATT | GCAGCTGCAATAC | ATGATTCCAGCATT |
| | ACATGGTT | GGGGCA | ACATGGT | GGGGCA |
| | (SEQ ID NO: 226) | (SEQ ID NO: 227) | (SEQ ID NO: 228) | (SEQ ID NO: 227) |

The inventors observed that the environment influenced the development of purple color in cannabis leaves and flowers. The same training population was also grown in a polytunnel in a plot adjacent to the field experiment. The plants in the polytunnel were scored for purple color at time of a harvest on a scale from 1-9, where 9 is most purple. The inventors found the average color score of plants grown in the poly tunnel was 3.08. The average color score of plants grown in the open field was 3.5. The inventors determined that in the polytunnel the purple color in cannabis plants developed less strongly and in some cases failed to appear. In contrast, in the field grown cannabis plants purple color developed more strongly. From these results the inventors determined that environment influences the development of purple color in cannabis. This can complicate the correlation between phenotype and genotype in diverse environments. This is not unexpected, as stress has been reported to influence the accumulation of anthocyanin in a broad range of plant species. Different environmental conditions existed between the 2020, 2021 and 2022 field experiments. However, in each experiment there was strong evidence of the presence of a major QTL influencing the purple color or lack of purple color in cannabis at position 70000000-78000000 on chromosome NC_044377.1.

Due to the environmental effect on purple color emergence, validation of this phenotype can be complicated. To demonstrate the use of the markers in the selection of plants for the green or purple color, the inventors used all SNP markers in Table 9 together to demonstrate the predictive power of the markers in this region.

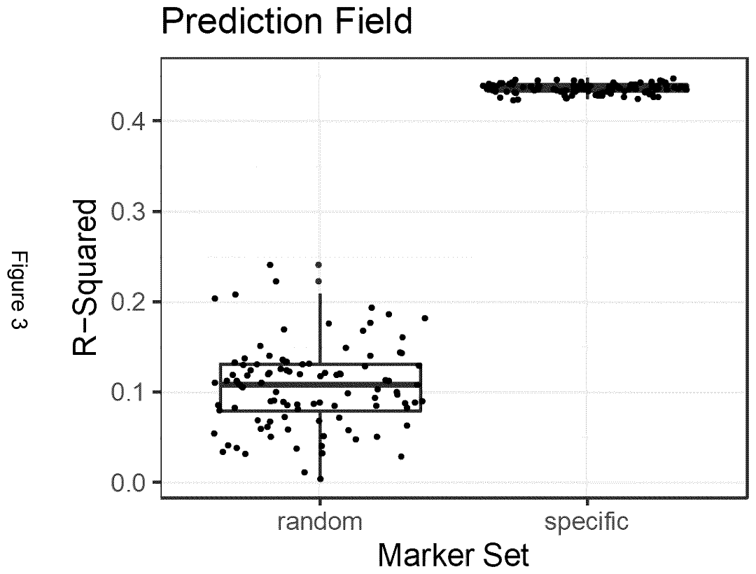

The inventors demonstrated the use of a genomic selection model and tested this on the training population of mixed lineage green and purple plants diverse cannabis plants described above. The model based on the markers listed in Table 9, tests if the markers together improve the prediction power for the selection of purple color plants compared to 25 randomly selected markers. The inventors performed a multiple regression analysis with the allele as variable and purpleness as target using the random forest algorithm implemented in the ranger package (v 0.12.1, Wright and Zieger 2017). The resulting R squares are derived from the comparison of the predictions from the developed model with the measured phenotype of the training population (FIG. 3).

The approach tested 100 permutations for the specific markers and 100 permutations of the 25 random markers resampled for each permutation, with each dot in FIG. 3 representing one of those permutations. The results of the genomic selection model demonstrate that the specific markers in this region greatly improve the accuracy of selecting for purple color, with R-squared of ˜0.45, in comparison with the use of random markers, with R-squared of ˜0.1.

1. A method for characterizing a Cannabis spp. plant with respect to a purple color trait, the method comprising the steps of:
(i) genotyping at least one plant with respect to a purple color QTL by detecting one or more polymorphisms associated with the purple color trait as defined in any one of Tables 1 to 4 and 7 to 8; and
(ii) characterizing the at least one plant with respect to the purple color QTL based on a genotype at the one or more polymorphisms.

2. The method of claim 1, wherein the one or more polymorphisms are selected from the group consisting of “common_4519”, “common 4525”, “common 4500”, “common 4513”, “rare_551”, “common 4487”, “common 4504”, “common 4516”, “rare_556”, “GBScompat_common_878”, “GBScompat_rare_165”, and combinations thereof, as defined in any one of Tables 1 to 4 and 7 to 8.

3. The method of claim 1, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

4. The method of claim 3, wherein the molecular markers are for detecting polymorphisms at regular intervals within the purple color QTL such that recombination can be excluded.

5. The method of claim 3, wherein the molecular markers are for detecting polymorphisms at regular intervals within the purple color QTL such that recombination can be quantified to estimate linkage disequilibrium between a particular polymorphism and a purple color phenotype.

6. The method of claim 3, wherein the molecular markers are designed based on a context sequence for the polymorphism in Tables 1 to 4 or 10 or are selected from the primer pairs as defined in Table 5 or 11.

7. The method of claim 1, wherein the purple color QTL is a quantitative trait locus having a sequence that corresponds to nucleotides 68717484 to 77040783 of NC 044377.1 with reference to the CS10 genome and is defined by one or more polymorphisms associated with purple color as defined in any one of Tables 1 to 4 and 7 to 8, or a genetic marker linked to the QTL.

8. A method of producing a Cannabis spp. plant having a purple color trait of interest, the method comprising the steps of:
(i) providing a donor parent plant having in its genome a purple color QTL characterized by one or more polymorphisms associated with the purple color trait of interest as defined in any one of Tables 1 to 4 and 7 to 8;
(ii) crossing the donor parent plant having the purple color QTL with at least one recipient parent plant to obtain a progeny population of cannabis plants;
(iii) screening the progeny population of cannabis plants for the presence of the purple color QTL; and
(iv) selecting from the progeny population one or more progeny plants having the purple color QTL, wherein a mature plant obtained from the one or more progeny plants displays the purple color trait of interest.

9. The method of claim 8, further comprising:
(v) crossing the one or more progeny plants with the donor recipient plant; or
(vi) selfing the one or more progeny plants.

10. The method of claim 8, wherein the screening comprises genotyping at least one plant from the progeny population with respect to the purple color QTL by detecting one or more polymorphisms associated with the purple color trait of interest as defined in any one of Tables 1 to 4 and 7 to 8.

11. The method of claim 8, wherein the method comprises a step of genotyping the donor parent plant with respect to the purple color QTL by detecting one or more polymorphisms associated with the purple color trait of interest as defined in any one of Tables 1 to 4 and 7 to 8, prior to step (i).

12. The method of claim 10, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.

13. The method of claim 12, wherein the molecular markers are for detecting polymorphisms at regular intervals within the purple trait QTL such that recombination can be excluded or such that recombination can be quantified to estimate linkage disequilibrium between a particular polymorphism and the purple color trait of interest.

14. The method of claim 12, wherein the molecular markers are designed based on a context sequence for the polymorphism in Tables 1 to 4 or 10 or are selected from the primer pairs as defined in Table 5 or 11.

15. The method of claim 8, wherein the purple color QTL is a purple color presence QTL or a purple color absence QTL.

16. The method of claim 8, wherein the one or more polymorphisms are selected from the group consisting of “common_4519”, “common_4525”, “common 4500”, “common 4513”, “rare_551”, “common_4487”, “common_4504”, “common 4516”, “rare_556”, “GBScompat_common_878”, “GBScompat_rare_165”, and combinations thereof, as defined in any one of Tables 1 to 4 and 7 to 8.

17. The method of claim 8, wherein the purple color QTL is a quantitative trait locus having a sequence that corresponds to nucleotides 68717484 to 77040783 of NC 044377.1 with reference to the CS10 genome and is defined by one or more polymorphisms associated with purple color as defined in any one of Tables 1 to 4 and 7 to 8, or a genetic marker linked to the QTL.

18. A method of producing a Cannabis spp. plant comprising a purple color trait of interest, the method comprising introducing a purple color QTL characterized by one or more polymorphisms associated with the purple color trait of interest as defined in any one of Tables 1 to 4 and 7 to 8 into a Cannabis spp. plant, wherein said purple color QTL is associated with the purple color trait of interest in the plant.

19. The method of claim 18, wherein introducing the purple color QTL comprises crossing a donor parent plant having the purple color QTL characterized by one or more polymorphisms associated with the purple color trait of interest with a recipient parent plant.

20. The method of claim 18, wherein introducing the purple color QTL characterized by one or more polymorphisms associated with the purple color trait of interest comprises genetically modifying the Cannabis spp. plant.

21. The method of claim 18, wherein the purple color QTL is a quantitative trait locus having a sequence that corresponds to nucleotides 68717484 to 77040783 of NC 044377.1 with reference to the CS10 genome and is defined by one or more polymorphisms associated with purple color as defined in any one of Tables 1 to 4 and 7 to 8, or a genetic marker linked to the QTL.

22-24. (canceled)

25. A Cannabis spp. plant comprising a purple color QTL characterized by one or more polymorphisms associated with a purple color trait of interest as defined in any one of Tables 1 to 4 and 7 to 8.

26-34. (canceled)

35. The method of claim 11, wherein the genotyping is performed by PCR-based detection using molecular markers, sequencing of PCR products containing the one or more polymorphisms, targeted resequencing, whole genome sequencing, or restriction-based methods, for detecting the one or more polymorphisms.
